(19) United States
(12) Patent Application Publication
(10) Pub. No.: US 2013/0157887 A1
(43) Pub. Date: Jun. 20, 2013

(54) DISCRETE STATES FOR USE AS BIOMARKERS

(71) Applicants: Manfred Beleut, Winterthur (CH); Peter Schraml, Basel (CH); Holger Moch, Urdorf (CH); Michael Baudis, Zurich (CH); Philip Zimmermann, Grenchen (CH); Wilhelm Gruissem, Forch (CH); Karsten Henco, Dusseldorf (DE)

(72) Inventors: Manfred Beleut, Winterthur (CH); Peter Schraml, Basel (CH); Holger Moch, Urdorf (CH); Michael Baudis, Zurich (CH); Philip Zimmermann, Grenchen (CH); Wilhelm Gruissem, Forch (CH); Karsten Henco, Dusseldorf (DE)

(21) Appl. No.: 13/697,419

(22) PCT Filed: May 12, 2011
(86) PCT No.: PCT/EP2011/057691

§ 371 Date: Mar. 11, 2013

Related U.S. Application Data
(60) Provisional application No. 61/334,318, filed on May 13, 2010.

(30) Foreign Application Priority Data
May 13, 2010 (EP) 10162783.4

(51) Int. Cl. C12Q 1/68

(52) U.S. Cl. CPC C12Q 1/6837; C12Q 1/68
USPC 506/9; 435/6.1

(57) ABSTRACT

The present invention describes the use of discrete states and signatures for classifying samples.

### BACKGROUND OF THE INVENTION

### Field of the Invention

Before the advent of molecular biology and medicine, diseases have largely been classified on the basis of their phenotypic characteristics. This, of course, means that a disease can only be diagnosed when phenotypic characteristics become apparent which may occur at a rather late stage of disease development. Further, it is nowadays understood that similar phenotypes may result from different molecular mechanisms. A strictly phenotype-based therapy may therefore be useless if the therapeutic approach taken does not address the right underlying mechanism.

As an example, breast cancer may develop by different molecular mechanisms which lead to the same appearance in terms of tumor formation. One such mechanism will involve up-regulation of Her2 while others will not. Therapy with the antibody Herceptin® which addresses overexpression of Her2 will therefore only help patients which are afflicted correspondingly. If one does not understand at least to some degree the molecular mechanisms underlying a disease, a chosen therapy may not prove effective.

Molecular biology and medicine therefore aim at deciphering the molecular basis of disease development. A better understanding of the molecular basis of a disease will help detecting imminent or ongoing disease development early on and will allow medical practitioners adjusting their therapy early on or developing alternative treatment approaches. For example, if one knows that Herceptin® will be effective only in a specific group of patients, one can pre-select these patients and treat them accordingly. Further, if one realizes that different diseases result at least to some degree from the same mechanism, one can consider a drug, which has originally been developed for one disease only also for treatment of the other diseases. This, of course, requires that molecular markers, which are frequently designated as biomarkers, are at hand being characteristic for the disease in question and relating to relevant mechanisms, relevant clinical endpoints and relevant criteria to select proper treatment. Such markers may be found on the DNA, the RNA or the protein level.

In the case of monogenetic diseases, using molecular markers as a diagnostic tool is relatively straightforward as one can use the aberration on the DNA level to predict whether the disease will develop with a certain probability or not. For example, tri-nucleotide expansions on the DNA level may be used to predict whether an individual will develop Huntington Chorea. Similarly, mutations in the Survival of Motor Neurons gene can be used to predict whether an individual will develop Spinal Muscular Atrophy.

Since the beginning of molecular understanding of tumor diseases there is a desire to define molecular markers associated with tumorigenesis, malignancy, progression, metastasis formation, responsiveness to treatment, survival times and other functional properties important for clinicians and for the development of efficient therapies. A number of useful markers were identified, first of all pathological markers for the inspection of samples such as derived from tissue sections (large sections, fine needle biopsies), body fluids, smears (blood, feces, sputum, urine) or hair samples. A number of markers got identified such as markers of inflammation or ongoing apoptosis, markers of metabolic properties or molecular markers derived from mechanistic understanding of tumor induction, induced by deregulated balances between oncogenes such as Ras, Myc, CDKs and tumor suppressor genes such as p16, p27 or p53 (see e.g. Hanahan & Weinberg in “The Hallmarks of Cancer” (2000).

Specific understanding of tumor development mechanisms such as uncontrolled cellular growth, senescence and apoptosis evasion, such as extravasation, invasion, and evasion of immune responses have further accentuated the tumor suppressor gene hypothesis.

However, the vast majority of diseases such as hyper-proliferative disease including cancers does not result from mono-genetic causes but are due to aberrant complex molecular interactions.

Cancer, for example, is considered as a prime example for multi-factorial diseases which arise from subtle to severe deregulation of complex molecular networks. In most cases, these diseases do not develop from a single gene mutation but rather result from the accumulation from mutations in various genes. Each single mutation may not be sufficient in itself to start disease development. Rather, accumulation of mutations over time seems to increasingly deregulate the complex molecular signaling networks within cells. In these cases, disease development has therefore usually been considered to be a gradual continuous process which cannot be characterized by key events. As a consequence thereof, it is commonly assumed that such diseases cannot be diagnosed or classified by a single biomarker but by a group of markers which ideally would reflect in a simplified manner the complex molecular mechanisms underlying the disease.

Despite the large amount of molecular information available from many human cancers, current cancer research mainly still focuses on single, frequently altered chromosomal loci ideally harboring tumor type-specific biomarker candidates with drug target potential such as enhanced angiogenesis lead to the understanding of tumor promoting roles of the Her-receptor family and its ligands and related mutants. Some of those attempts indeed led to certain useful markers for the selection of tumor therapies (Herceptin® treatment for patients of amplified Her-2 receptors).

All these results mainly resulted from a maximum of expert knowledge. The general and common assumption is that tumors must be different from normal tissues due to above mentioned target expression. The majority of studies, often linked to pathologic parameters (such as tumor subtypes, grade or staging), therefore address their focus on the investigation of single targets. Even though their role in certain pathways and their binding partners may become evident in appropriate cell lines or mouse models their specific role as part of an entire network remains unclear.

The human genome project together with all its spin-off projects such as analysis of individual genome varieties between individuals or just individual cells affected by a disease, analyses of respective transcriptomes, proteomes etc. were assumed to directly provide a large variety of useful biomarkers. Interestingly, most of these approaches have tried again to link the phenotypic differences observed for disease with distinct molecular pathways.

There are e.g. a number of types and subtypes of diseases, obviously associated with some clearly differentiable markers on the level of e.g. organs such as lung cancer or prostate cancer or e.g. cell types. The common concept for identifying biomarkers is to link such phenotypes to distinct combinations of biomarkers which then allow diagnosing the specific subtype of disease, which displays the respective phenotype. Such approaches, for example, try identifying distinct proteome expression patterns for small cell lung cancer tissues or non-small lung cancer tissues of afflicted patients vs. healthy individuals and to then use such expression patterns to diagnose patients in the future. Interestingly, these approaches frequently do not look at linking clinically relevant parameters such as survival time with markers.

However, the wealth and complexity of data have hindered clear cut identification of such patterns to some extent.

There is thus a continuing need for tools allowing classification of diseases on the molecular level and provision of biomarkers which can be used for e.g. diagnostic purposes.

### SUMMARY OF THE INVENTION

It is one objective of the present invention to provide new types of markers, which are suitable and specific for classifying diseases, preferably with clear correlation to clinically or pharmacologically relevant endpoints.

It is also an objective of the present invention to provide methods for detecting markers which are suitable and effective for classifying diseases, preferably with clear correlation to clinically or pharmacologically relevant endpoints.

These and other objectives as they will become apparent from the ensuing description are attained by the subject matter of the independent claims. The dependent claims relate to some of the preferred embodiments of the invention.

The present invention provides a strategic and direct approach to global and functional biomarkers of clinical relevance for essentially all kinds of tumors and potentially non-tumor diseases, too. With the present finding of tumors being associated with discrete stable or meta-stable states, one is now able to define methods allowing the skilled person to not only identify and prove the existence of such discrete states for any kind of tumor but to assign such states with descriptors and signatures associated with such states. In addition, the technology allows to identify a minimum of those descriptors which unequivocally identify and discriminate each such discrete state from alternative states in a given tumor cell sample.

The understanding of such states also allows identifying those descriptors with a large dynamic range for quantitative measurement and ease of experimental access.

The invention is thus based on the surprising finding that diseases can be characterized by discrete states, which reflect the underlying molecular mechanisms. Interestingly, these discrete states are distinct from one another so that disease development does not seem to be characterized by a continuous process. Rather, a discrete state seems to be maintained until a certain threshold level is reached when a switch to another discrete state occurs. Further, it seems that the discrete states can be linked to clinically and pharmacologically important parameters. However, they do not necessarily seem to coincide with standard histological classification schemes.

Each discrete state can be described by way of different signatures. A signature is a pattern reflecting the qualitative and/or quantitative appearance of at least one descriptor. Preferably, a signature is a pattern reflecting the qualitative and/or quantitative appearance of multiple descriptors. Descriptors may in principle be any testable molecule, function, size, form or other parameter that can be linked to a cell. Descriptors may thus be e.g. genes or gene-associated molecules such as proteins and RNAs. The expression pattern of such molecules may define a signature.

These findings of the invention can be used for various diagnostic, prognostic and therapeutic purposes. They may also be used for research and development on and of new treatments for diseases such as hyper-proliferative diseases.

In one aspect, the invention thus relates to at least one discrete disease-specific state for use as a diagnostic and/or prognostic marker in classifying samples from patients, which are suspected of being afflicted by a disease such as a hyper-proliferative disease. The invention further relates to at least one discrete disease-specific state for use as a diagnostic and/or prognostic marker in classifying cell lines of a disease such as a hyper-proliferative disease. The invention also relates to at least one discrete disease-specific state for use as a target for development, identification and/or screening of pharmaceutically active compounds.

As discrete disease specific states may be determined by signatures, the invention in one embodiment relates to at least one signature for use as a diagnostic and/or prognostic marker in classifying samples from patients which are suspected to be afflicted by a disease such as a hyper-proliferative disease. The invention also relates to at least one signature for use as a diagnostic and/or prognostic marker in classifying cell lines of a disease such as a hyper-proliferative disease. The invention further relates to at least one signature for use as a read out of a target for development, identification and/or screening of pharmaceutically active compounds.

In some embodiments, the invention relates to methods of diagnosing a disease such as a hyper-proliferative disease by making use of signatures and discrete disease-specific states.

The invention also relates to methods of determining the responsiveness of a test population suffering from a disease such as a hyper-proliferative disease towards a pharmaceutically active agent by making use of signatures and discrete disease-specific states.

Further, the invention relates to methods of predicting the responsiveness of patients suffering from a disease such as a hyper-proliferative disease in clinical trials towards a pharmaceutically active agent by making use of signatures and discrete disease-specific states.

The invention also relates to methods of determining the effects of a potential pharmaceutically active compound by making use of signatures and discrete disease-specific states.

Aside from the specific uses of discrete disease specific states and signatures, the invention also relates to methods for identifying signatures and discrete disease specific states in samples which may be derived from patients or which may e.g. be cell lines.

All of these embodiments of the invention can be used in the context of diseases including hyper-proliferative diseases such as cancer and preferably in the context of renal cell carcinoma.

### DESCRIPTION OF THE FIGURES

FIG. 1 A) Regional genomic CNAs in RCC shown as percentage of analyzed cases. Imbalance frequencies are shown as percentages on −50 to 50 scale for chromosomes 1 to 22 (every second chromosome is indicated for orientation). Upper panel: depiction of the overall CNAs in the 45 study cases, genomic gains are depicted above the zero line, genomic losses are depicted below the zero line. Lower Panel: published chromosomal and array CGH RCC data accessible through the Progenetix database (472 cases). CNVs were not filtered from the study case data besides application of a 100 kb size limit. Genomic gains are depicted above the zero line, genomic losses are depicted below the zero line. B) The PANTHER classification output matches 557 genes previously identified by SNP to 76 superior biological processes. The 4 dominating “networks” are numbered. The Y-axis indicates the number of genes found for a network on a scale of 0 to 38. Note: To increase matching efficacy, the initial 769-gene list was simultaneously run against “Pubmed” and “Celera” databank. Therefore, divergent output numbers are shown in this bar chart (ex. Genes/Total genes).

FIG. 2 Hierarchical clustering of HG-U133A microarray probe sets representing genes from the Angiogenesis (A), Inflammation (B), Integrin (C), and Wnt (D) “pathways” as annotated by PANTHER, across a set of 147 microarrays from our RCC experiment. Blue: relative increase-, white: -decrease in gene expression. For each “pathway”, up to four probe set clusters (boxes) were selected, which were strongly representative for the overall partitioning of the RCC samples. The clusters were identified by the SAM software. Each row designates the genes analyzed for each pathway. Each line represents the samples analyzed. The densograms next to the lines and above the rows indicate the grouping of the samples and genes.

FIG. 3 Identification of RCC groups A, B, C and cell lines. Two-way hierarchical clustering of Affymetrix expression microarray data of 147 RCC samples against 92 genes assembled from clustering the most significant biological processes. Blue: relative increase-, white: -decrease in gene expression. The clusters were identified by the SAM software. Each row designates the genes analyzed. Each line represents the samples analyzed. The densograms next to the lines and above the rows indicate the grouping of the samples and genes.

FIG. 4 Heatmaps of RCC group- and different cancer type-specific signatures. Yellow or red (absolute values) indicate relative increase-, blue or green (ratios of tumors vs. normal tissues) relative decrease in gene expression. The areas in which overexpression is observed are indicated by arrows. A) Gene expression of the about 50 best classifiers of tumor type B against A and C across a subset of types A, B and C tumors (left picture). Comparative meta-analysis of these genes in GENEVESTIGATOR revealed multiple other tumor types with identical expression signatures (right picture). Rows indicate the samples, lines indicate the genes. The first 34 lines (top to bottom, left and right picture) correspond to the genes in the order of table 1. The last 16 lines (top to bottom, left and right picture) correspond to the genes in the order of table 2. The first 16 rows (left to right, left picture) correspond to samples of which 7 were papillary RCCs and 9 were clear cell RCC. All of them are of state B. The next 24 rows (left to right, left picture) correspond to samples of which 7 were papillary RCCs and 17 were clear cell RCC. All of them were either state A or C. The next 20 rows (left to right, right picture) correspond to samples of which 4 were kidney cancers and RCCs, 3 were breast cancers, 1 was multiple myeloma, 1 was adnexal serous carcinoma, 4 were anaplastic large cell lymphoma, 1 was oral squamous cell carcinoma, 1 was gastric cancer, 1 was colorectal adenoma, 4 were angioimmunoblastic T-cell lymphoma. These were either state A or C. The next 8 rows (left to right, right picture) correspond to samples of which 1 was a gastric tumor, 6 were an ovarian tumor and 1 was an aldosterone-producing adenoma. All of them were state B. In the left picture, the upper left part and lower right part indicate overexpression. The lower left part and upper right part indicate reduced expression. The dashed line indicates the left, right, upper and lower parts. In the right picture, the upper left part and lower right part indicate reduced expression. The lower left part and upper right part indicate overexpression. The dashed line indicates the left, right, upper and lower parts. B) Gene expression of the 24 best classifiers of tumor type A against C across a subset of types A and C tumors (left picture), and correlated other tumors (right picture) as identified in GENEVESTIGATOR. All signatures are cancer-specific and not detectable in corresponding “normal” tissues. Rows indicate the sample, lines indicate the genes. The first 5 lines (top to bottom, left and right picture) correspond to the genes in the order of table 3. The first two lines represent different isoforms of the same gene (RARRES 1). The last 19 lines (top to bottom, left and right picture) correspond to the genes in the order of table 4. The first 9 rows (left to right, left picture) correspond to samples all of which were clear cell RCCs. All these are state A. The next 15 rows (left to right, left picture) correspond to samples of which 7 were papillary RCCs and 8 were clear cell RCC. These are state C. The next 4 rows (left to right, right picture) correspond to samples of which 2 were kidney cancers and 2 were thyroid cancers. These are state A. The next 12 rows (left to right, right picture) correspond to samples, of which 2 were cervical squamous cell carcinoma, 1 was adenocarcinoma, 1 was adnexal serous carcinoma, 3 were bladder cancers and 5 were breast cancers. These are state C. In the left picture, the upper left part and lower right part indicate reduced expression. The lower left part and upper right part indicate overexpression. The dashed line indicates the left, right, upper and lower parts. In the right picture, the upper left part and lower right part indicate reduced expression. The lower left part and upper right part indicate overexpression. The dashed line indicates the left, right, upper and lower parts. C) Hierarchical clustering of 40 RCC samples across all probe sets of the HG-U133A array, identifying the 3 groups which are indicated by arrows as state A, B or C (left). Hierarchical clustering of the 40 (colour coded) RCC samples based on expression signal values from 662 probe sets representing a subset of the 769 genes identified from the SNP array analysis, unravelling the 3 RCC groups (right). The densogram reflects the relationship between the 40 RCC samples. D) Kaplan-Meier analysis of tumour-specific survival in 176 RCC patients; grouped in A (high MVD, DEK and MSH positive), B (MSH6 negative) and C (low MVD, DEK and MSH positive) (log rank test: p<0.0001). The y-axis indicates the percentage of survivors in 0% to 100% scale. The x-axis indicates the average survival time on a 0 to 100 month scale.

FIG. 5 RCC test-TMA with antibody staining combinations of the markers CD34, DEK and MSH6 used to define group A, B and C. Magnified images illustrate specific staining of endothelial micro vessels (CD34) and nuclei of tumor cells (DEK and MSH6).

FIG. 6 Shows the analysis of RCC testing with different antibodies.

FIG. 7 An evolutionary driven molecular classification model for renal cell cancer.

### DETAILED DESCRIPTION OF THE INVENTION

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

In the context of the present invention the terms “about” or “approximately” denote an interval of accuracy that the person skilled in the art will understand to still ensure the technical effect of the feature in question. The term typically indicates deviation from the indicated numerical value of ±10%, and preferably of ±5%.

As mentioned above, previous attempts in finding diagnostic tools for disease characterization have assumed that disease development is a continuous process and have tried to link different primarily histological phenotypes of e.g. cancers such as lung cancer with specific expression patterns assuming that the different detectable phenotypes reflect continuous and progressive disease development.

The present invention is instead based on the finding that it seems that diseases such as hyper-proliferative diseases can comprehensively be described by a limited set of discrete disease-specific states which do not necessarily correlate with established histological characterization of different subtypes of such a hyper-proliferative disease but which can be linked to clinically relevant parameters such as survival time. Without wanting to be bound to a specific scientific theory or expert knowledge, it is hypothesized that a disease is characterized by switching to discrete disease-specific states. This suggests that de-regulation of regulatory networks within a cell can occur to a certain a threshold level without the overall discrete state being affected. However, once the threshold level has been exceeded cells seem to switch to another specific discrete state. These states can therefore be considered as stable or meta-stable in that they may allow for a certain degree of variation before they may switch. We understand a discrete state to reflect the flow and extent of interactions between and within different regulatory networks. As cells seem to switch to different discrete states, such a switch seems to indicate a major re-arrangement of the flow and extent of interactions between and within different regulatory networks, which may lead to a changed aggressiveness of a disease and which may also help explaining why different discrete states can be linked to e.g. different average survival times.

Interestingly, there are discrete states that can be found in different types of hyper-proliferative diseases such as renal cell carcinoma or ovarian cancer, which may indicate that at least some forms of these diseases involve comparable molecular mechanisms. Further, there may be discrete states that can be found only within a specific hyper-proliferative disease.

The extent and flow of interactions between and within such different regulatory networks may be detectable by e.g. the expression level of e.g. proteins within such regulatory networks. The molecular entities, which are looked at can be designated as descriptors. The pattern, which is detected for a set of descriptors, can be considered as a signature. In the aforementioned example, the signature will be the expression pattern of proteins, which function as the descriptors. Of course, one may chose different types of descriptors and different types of signatures. One may thus look at expression levels of genes on the RNA level. One may look at the regulation of miRNAs and one may even look at the qualitative distribution of descriptors such as the cellular localization of certain factors or the shape of a cell. One may use a given set of descriptors of the same type of molecules (e.g. mRNAs) to define signatures with the different signatures reflecting e.g. different expression patterns or one may use a given set of descriptors which are a group of different molecules (such as mRNAs, proteins and miRNAs). It is thus important to note that according to the invention's logic a discrete state can be correlated to different signatures. As single signature will, however, define one discrete state only.

It follows from the invention as laid out hereinafter that the same discrete state can be characterized through different signatures.

As illustrated hereinafter for renal cell carcinoma (RCC), such discrete disease-specific states can be linked to medically important parameters such as average survival times. Interestingly, however, the discrete disease-specific states do not necessarily correlate with common histological classification schemes meaning that e.g. papillary RCCs of different patients may be characterized by different discrete molecular states and that the patients may thus have different survival expectations even though their cancers have been classified as comparable by histological standards. Moreover, it has been found that some of the discrete molecular states found for RCC can also be detected in other cancer types suggesting that different cancers, which are usually considered being unrelated in fact result at least to some extent from the same molecular interactions that define a discrete state.

Thus, a novel interpretation of carcinogenesis is suggested, which aims at a molecular de novo classification of tumors. This de novo classification lead to the identification of discrete disease specific states and signatures. The signatures were initially dissected in renal cell carcinoma (RCC) as a model, unbiased from current clinico-pathological (i.e. tumor stage, subtype, differentiation grade, tumor-specific survival), genetic (i.e. allelic gain/increased “oncogene” expression, allelic loss/decreased “tumor suppressor gene” expression) and biological (i.e. von Hippel-Lindau protein regulated pathways) valuations.

The finding that diseases such as hyper-proliferative disease can be characterized by different discrete disease-specific states, which may be present in different types of diseases, has important implications.

The discrete disease-specific state(s) may be used to classify patients and samples thereof as falling within distinct groups. As the discrete disease-specific state can moreover be linked to clinically important parameters such as survival time or responsiveness to distinct drugs, this will help selecting therapeutic regimens. The discrete molecular state(s) may thus be used as diagnostic and/or markers providing a new way of classifying tumors into clinically relevant subgroups e.g. subgroups of RCC, ovarian cancer, breast cancer etc.

A lot of projects for the development of novel pharmaceuticals suffer from insufficient differentiation from existing therapies, non-conclusive statistical data or a need for enormously high numbers of patients in Phase II or Phase III demanding for multimillion dollar investments and extensive time periods. If, however, a drug can be shown to act preferentially only in a selected group of patients which suffer from e.g. a subtype of lung cancer and which are characterized by the same discrete disease-specific state of interacting molecular networks, then this drug may be tested in other patients which suffer from a different disease, but are characterized by the same discrete molecular state. It can be expected that such clinical trials will give statistically reliable results for much smaller patient groups. In fact, one may be able to show that treatment is effective where large scale clinical trial could not give such results because the large number of non-responders will avoid any statistically meaningful interpretation of the results.

The discrete states thus provide a stratifying tool for the testing of pharmacological treatments as it allows grouping of patients for clinical trials. Assuming a drug candidate is identified which is expected or hoped to positively influence the critical parameter of survival time substantially, this needs to be proven by clinical trials in order to receive FDA approval. Future drugs will likely focus on mechanistic intervention. If the mechanistically active drug is successful for the clinical end point parameter “survival time”, it probably interacts selectively with mechanisms linked to the parameter “survival time”. These mechanistic subgroups are exactly those defined by e.g. the discrete molecular states enabled by this invention. It is thus fair to believe, that most probably one subgroup of patients reacts positively to a different degree than another subgroup does. Knowledge of this patient cohort-specific imbalance is of utmost importance for the industry seeking approval for a drug, important to know for the physician to choose the optimum regimen and for the payors to spend money most efficiently on patients with promise of therapeutic success. Any definition of a subgroup reacting with maximum relative effect in terms of prolonged life expectancy improves the chance for FDA registration.

The knowledge about discrete disease-specific states may also allow using these states as targets during development of pharmaceutical products. For example, different discrete specific disease states may be linked to clinically relevant parameters such as survival time or response rate to a certain drug. If an agent is shown to switch the discrete disease-specific state in a sample or in a cell line from a state, which is linked to short survival time, into a state with long survival time, such a switch may be used as an indication that the agent may be therapeutically effective in treating the disease in question. Thus, assays can be designed which make use of the correlation between a discrete disease-specific state and e.g. the associated clinical parameter.

Further, knowing that discrete disease-specific states exist as such enables one now to identify new discrete disease-specific states. For example, the present invention shows that RCCs can be roughly characterized by three different discrete disease-specific states. Some of these discrete disease-specific states are shown to be present in cancers different from RCC such as e.g. ovarian cancer in addition. However, not all ovarian cancers can be linked to the discrete states, which were found for RCCs meaning that different discrete disease-specific states should be identifiable for ovarian cancer. In this context the invention also provides methods for identifying discrete molecular states or statistically excluding novel discrete disease specific states of a substantial subset of patients. For example, the invention shows that all cases of RCC can be attributed to three distinct discrete disease specific states.

The logic of these methods can also be used to define discrete substates within discrete states and further discrete substates within discrete substates which for ease of nomenclature may be designated as discrete level. This discrete substates and discrete level may allow describing a disease at an even finer level.

The specific discrete disease-specific states as identified herein can thus be used to not only characterize RCCs, but also to characterize other cancers or diseases in general. Further, they can provide guidance whether other discrete disease-specific states will exist in these other diseases.

The invention further provides methods for identifying such discrete disease-specific states as such as well as methods for identifying signatures of descriptors, which can be used to detect a discrete disease-specific state. For RCC, the invention in fact provides a list of gene descriptors, the expression pattern of which (i.e. the signature) allows classifying RCCs according to the average survival time.

The fact that one now knows that discrete disease-specific states exist and drive disease development in all its aspects allows one to identify signatures of descriptors, which can then be used in a diagnostic test to classify diseases such as different types of hyper-proliferative diseases. These signatures of descriptors thus serve as a read-out for the classification of a disease or its subtype.

The invention and its embodiments will now be described in greater detail. For a better understanding of the following definitions, a rough outline of the findings in the context of RCCs is given. The data, which led to the identification of discrete disease specific states, will then be discussed in further detail later on.

It was found that the overall majority of RCCs irrespective of their histological characterizations as papillary, chromophobe and clear cell RCC can be classified into three discrete disease-specific states which are indicative of a long, intermediate and short survival time. The discrete disease-specific states are thus likely reflecting the aggressiveness of the tumor. The read-out for these three discrete molecular states which are designated hereinafter as A, B and C are the expression patterns, i.e. the signatures of a limited set of descriptors, i.e. genes. The same signatures, i.e. expression patterns of the same genes were then detected at least to some extent in other cancer types such as lymphoma, myeloma, breast cancer, colorectal cancer or ovarian cancer. This suggests that developing different hyper-proliferative diseases involves to at least some extent the same molecular mechanisms. Further, this finding suggests that different hyper-proliferative disease can be classified to some extent into the same discrete disease-specific stages. These states in turn allow a prognosis of the survival times of these different hyper-proliferative diseases. In order to identify the signatures and thus the discrete disease-specific states of RCCs an approach of hierarchical clustering of expression data was used which can be applied to identify further discrete disease-specific states in these different cancers or other diseases. It is key feature of this approach that it looks at descriptors from at least two different regulatory networks.

We will now provide definitions useful to understand the present invention and will then discuss the invention in more detail.

“State” means a stable or meta-stable constellation of a cell and/or cell population which is identified in at least two biological samples from at least two patients and which can be described by means of a single descriptor or multiple descriptors on the cellular or molecular level referenced against a standard state. As explained hereinafter, such state can be identified through analyzing descriptors from at least two regulatory networks. As explained hereinafter, such state can be characterized by at least one or various signatures or surrogate signatures.

“Substate” means a stable or meta-stable constellation of a cell within a state which is identified in at least two biological samples from at least two patients and which can be described by means of at least two descriptors on the cellular or molecular level referenced against a standard state. As explained hereinafter, such substate can be identified through analyzing descriptors from at least two regulatory networks. As explained hereinafter, such substate can be characterized by at least one or various signatures or surrogate signatures.

“Level” means a stable or meta-stable constellation of a cell within a substate which is identified in at least two biological samples from at least two patients and which can be described by a at least three descriptors on the cellular or molecular level referenced against a standard state. As explained hereinafter, such level can be identified by analyzing descriptors from at least two regulatory networks. As explained hereinafter, such level can be characterized by at least one or various signatures or surrogate signatures.

By definition, different states, substates and levels refer to different stabile and metastabile constellations of a cell meaning that these constellations are distinct from each other in terms of the kind and extent of molecules of at least two regulatory networks interacting within a cell. Different states, substates and levels can be characterized by a limited set of descriptors giving rise to different signatures. They may therefore also be designated a “discrete molecular state, substate or level”.

If a state, substate or level is indicative of a disease, it may be designated as “disease specific molecular state, substate, or level”. In certain instances, a disease specific state, substate, or level may be linkable to clinically relevant parameters such as survival rate, therapy responsiveness, and the like.

A state, substate or level, which can be found in healthy human or animal subjects may be designated as “healthy state, substate, or level”.

The term discrete disease specific state, substate or level preferably allows distinguishing different subtypes of a disease according to a new classification scheme which links the subtype being characterized by a discrete disease specific state, substate or level to clinically or pharmacologically important parameters.

The terms “clinical or pharmacological relevant parameter” preferably relate to efficacy-related parameters as they will be typically analyzed in clinical trials. They thus do not necessarily relate to a change in the histological appearance of a disease, but rather to important clinical end points such as average survival time, progression-free survival times, responsiveness to a certain drug, subjective patient- or physician-rated improvements making use established scale systems, tolerability, adverse events. The terms also include responsiveness towards treatment.

“Descriptor” means a measurable parameter on the molecular or cellular level which can be detected in terms of, but not limited to existence, constitution, quantity, localization, co-localization, chemical derivative or other physical property. A descriptor thus reports at least one qualitative and/or quantitative measuring parameter of, but not limited to existence, kinetic variation, clustering, cellular localization or co-localization of at least one specific mRNA, processing or maturation derivatives of at least one specific mRNA, specific DNA-motifs, variants or chemical derivatives of such motifs, such as but not limited to methylation pattern, miRNA motifs, variants or chemical derivatives of such miRNA motifs, proteins or peptides, processing variants or chemical derivatives of such proteins or peptides or any combination of the foregoing.

By way of example, a descriptor may be a protein the over- or underexpression of which can be used to describe a discrete disease-specific state, substate or level vs. a different discrete disease-specific state, substate or level or vs. the discrete healthy state, substate or level. If different proteins, i.e. different descriptors are analyzed for their expression behavior, the observed pattern of over- and/or underexpression for this set of descriptors gives a rise to a pattern, which may be designated as signature (see below). It is to be understood that different types of descriptors may be used to describe the same discrete state, substate and level. For example, a set of descriptors may comprise expression data for a first set of proteins, data on post-translational modifications of a second set of proteins and data for a group of miRNAs.

Preferred descriptors include genes and gene-related molecules such as mRNAs or proteins.

The “qualitative” detection of a descriptor refers preferably to e.g. determining the localization of a descriptor such as a protein, an mRNA or miRNA within e.g. a cell. It may also refer to the size and/or the shape of cell.

The “quantitative” detection of a descriptor refers preferably to e.g. determining the presence and preferably the amount of a descriptor within a given sample.

In a preferred embodiment the quantitative measurement of a descriptor relates to detecting the amount of genes and gene-related molecules such as mRNAs or proteins.

The pattern resulting from the analysis of this combined set of descriptors will then be considered to be a signature.

“Signature” means a pattern of a set of at least two experimentally detectable and/or quantifiable descriptors with the pattern being a characteristic description for a discrete state, substate and/or level.

“Surrogate signature” shall mean any kind of potential alternative signature suitable for characterizing the same discrete state, substate or level.

Signal transduction refers to the communication between molecules interacting outside, on and/or inside in order to provide a chemical or physical output signal in response to a chemical or physical input signal. It is thus used as common in the art.

The term “signal transduction chain” as it is commonly used in the arte refers to the full or complete series of molecules, which linearly interact with each other to convert a set of specific chemical or physical input signals into a set of specific or chemical output signals. Thus, linear signal transduction pathways have been defined to describe e.g. the step wise signaling from specific receptors such as integrins into the cell's nucleus. It is understood that different linear signal transduction chains can cross-communicate with each other or comprise regulatory mechanisms such as feed-back loops.

“Regulatory network” describes the multidimensional nature and kybernetics of linearly simplified signal transduction chains and their interactions. They thus define the set of molecules which may belong to different signal transduction pathways but which may contribute to biological processes such as inflammation, angiogenesis etc. the impairment of which may contribute to a disease in all its aspects.

Regulatory networks may preferably those, which are provided by the PANTHER software (Protein Analysis Through Evolutionary Relationships, see e.g. http://www.pantherdb.org, Thomas et al., Genome Res., 13: 2129-2141 (2003), (20, 21)). The PANTHER software when used at its standard parameters comprises 165 regulatory networks, which may also be designated as pathways.

The term “diseases” relate to all types of diseases including hyper-proliferative diseases. The term reflects the all stages of a disease, e.g. the formation of a disease including initial stages, the development of a disease including the spreading of a disease, the stages of manifestation, the maintenance of a disease, the surveillance of a disease etc.

The term “hyper-proliferative” diseases relates to all diseases associated with the abnormal growth or multiplication of cells. A hyper-proliferative disease may be a disease that manifests as lesions in a subject. Hyper-proliferative diseases include benign and malignant tumors of all types, but also diseases such as hyperkeratosis and psoriasis.

Tumor diseases include cancers such as such as lung cancer (including non small cell lung cancer), kidney cancer, bowel cancer, head and neck cancer, colo(rectal) cancer, glioblastom, breast cancer, prostate cancer, skin cancer, melanoma, non Hodgkin lymphoma and the like.

In particular, cancers considered are as defined according to the International Classification of Diseases in the field of oncology (see http://en.wikipedia.org/wiki/carcinoma). Such cancers include epithelial carcinomas such as epithelial neoplasms; squamous cell neoplasms including squamous cell carcinoma; basal cell neoplasms including basal cell carcinoma; transitional cell papillomas and carcinomas; adenomas and adenocarcinomas (glands) including adenoma, adenocarcinoma, linitis plastic, insulinoma, glucagonoma, gastrinoma, vipoma, cholangiocarcinoma, hepatocellular carcinoma, adenoid cystic carcinoma, carcinoid tumor, prolactinoma, oncocytoma, hurthle cell adenoma, renal cell carcinoma, grawitz tumor, multiple endocrine adenomas, endometrioid adenoma; adnexal and skin appendage neoplasms; mucoepidermoid neoplasms; cystic, mucinous and serous neoplasms including cystadenoma, pseudomyxoma peritonei; ductal, lobular and medullary neoplasms; acinar cell neoplasms; complex epithelial neoplasms including Warthin's tumor, thymoma; specialized gonadal neoplasms including sex cord-stromal tumor, thecoma, granulosa cell tumor, arrhenoblastoma, Sertoli-Leydig cell tumor; paragangliomas and glomus tumors including paraganglioma, pheochromocytoma, glomus tumor; nevi and melanomas including melanocytic nevus, malignant melanoma, melanoma, nodular melanoma, dysplastic nevus, lentigo maligna melanoma, sarcoma and mesenchymal derived cancers, superficial spreading melanoma and acral lentiginous malignant melanoma.

The term “sample” typically refers to a human or individual that is suspected to suffer from e.g. a hyper-proliferative disease. Such individuals may be designated as patients. Samples may thus be tissue, cells, saliva, blood, serum, etc.

The term “cell lines” will designate cell lines which are either primary cell lines which were developed from patients' samples or which are typically be considered to be representative for a certain type of hyper-proliferative diseases.

It is to be understood that all methods and uses described herein in one embodiment may be performed with at least one step and preferably all steps outside the human or animal body. If it is therefore e.g. mentioned that “a sample is obtained” this means that the sample is preferably provided in a form outside the human or animal body.

It will be first described how signatures can be identified in accordance with the invention. It is to be understood that a signature will be indicative of a discrete disease-specific state.

In principle, signatures and discrete disease-specific states can be identified by analyzing for the quality and/or quantity of descriptors from at least two different regulatory networks for a multitude of samples from either patients of a hyper-proliferative diseases or cell lines of a hyper-proliferative disease. This data is then analyzed for certain patterns by (i) grouping the data for the quality and/or quantity across descriptors and (ii) grouping samples or cell lines in a second step for similarities of the quality and/or quantity of descriptor across all potential descriptors.

The present invention in one embodiment thus relates to a method of identifying a signature and optionally at least one discrete disease-specific state being implicated in a disease, optionally in a hyper-proliferative disease comprising at least the steps of:

a. Testing for quality and/or quantity of descriptors of genes or gene associated molecules in disease-specific samples derived from human or animal individuals which are suspected of suffering from said disease or in cell lines of said disease;

b. Clustering the results obtained in step a.) comprising at least the steps of:
i. Sorting the results for each descriptor by its quality and/or quantity,
ii. Sorting the disease-specific samples or cell lines for comparable quality and/or quantity of descriptors across all descriptors;
iii. Identifying different patterns for common sets of descriptors;
iv. Allocating to each pattern identified in step a.)iii.) a signature;
v. Optionally allocating to each signature identified in step b.),iv.) a discrete disease-specific state.

For such methods, it can be preferred to detect the quantity such as the expression of descriptors such as mRNAs or proteins. However, one may also look at other properties of other descriptors such as localization and processing of miRNAs or post-translational modification of proteins. One may thus look e.g. at the localization, the processing, the modification, the kinetics, the expression etc. of descriptors. For the sake of clarity, the following embodiments will be discussed with respect to expression patterns of descriptors such as mRNAs or proteins as these descriptors shall allow for straightforward identification of signatures and their implementation for e.g. diagnostic and/or prognostic purposes. It is however to be understood that this focus on expression data serves an explanatory purpose and shall not be construed as limiting the invention to expression data.

The clustering step b.) may be e.g. a hierarchical clustering process as it is implemented in various software programs. A suitable software may be e.g. the TIGR MeV software (23) using Euclidian distance and average linkage. The software is used with its default parameters.

The clustering step may preferably be a “two way hierarchical” clustering approach wherein e.g. first genes, i.e. descriptors are sorted by their expression intensity and wherein then samples are sorted for a comparable expression across all genes, i.e. all descriptors. In more detail, a two way clustering may be performed by the software according to gene expression intensities and tumor similarities. As a result, those tumors with an overall similar gene expression profile reside adjacent to each other. The software is used with its default parameters with Pearson Correlation as distance measure and optimal Leaf Ordering.

If this approach is undertaken for e.g. all human genes across a sufficient number of samples, in principle signatures, i.e. patterns of e.g. expression data should appear for a given set of descriptors. The identification of such signatures can be performed using SAM (12). The software is used with its default parameters. If a pattern for a set of descriptors has been identified, one can cross-check the accuracy by using alternative software such GENEVESTIGATOR (10, 11). It is to be understood that for a set of given descriptors, the appearance of different signatures is tantamount to the presence of discrete disease-specific states at this level of resolution. In more detail, a two way clustering may be performed by the software according to gene expression intensities and tumor similarities. As a result, those tumors with an overall similar gene expression profile reside adjacent to each other. The software is used with its default parameters with Pearson Correlation as distance measure and optimal Leaf Ordering.

This general approach may be limited in practical terms by e.g. the number of samples available or the necessary computing power.

There are, however, means to overcome these limitations and to allow identification of signatures with higher accuracy and speed.

In a preferred embodiment, the invention therefore relates to a method of identifying a signature and optionally at least one discrete disease-specific state being implicated in a disease, optionally a hyper-proliferative disease comprising at least the steps of:

a. Testing for quality and/or quantity of descriptors of genes or gene associated molecules in disease-specific samples derived from human or animal individuals suffering from said disease or in cell lines of said diseases;

b. Clustering the results obtained in step a.) comprising at least the steps of:
i. Sorting the results for each descriptor by its quality and/or quantity,
ii. Sorting the disease-specific samples or cell lines for comparable quality and/or quantity of descriptors across all descriptors;
iii. Identifying different groups of descriptors which are differentially regulated across said disease-specific samples or cell lines;

c. Combining the descriptors which are identified in step b.)iii.) wherein the quality and/or quantity of said descriptors disease-specific samples or cell lines are already known from step a.);

d. Clustering the results obtained in step c.) comprising at least the steps of:
i. Sorting the results for each descriptor of step c.) by its quality and/or quantity,
ii. Sorting the disease-specific samples or cell lines for comparable quality and/or quantity of descriptors across all descriptors;
iii. Identifying different patterns for the set of descriptors obtained in step c.);
iv. Allocating to each pattern identified in step d.)iii.) a signature;
v. Optionally allocating to each signature identified in step d.),iv.) a discrete disease-specific state.

This approach differs from the above embodiment in that the obtained data is clustered twice according to the same sorting principle. Thus in the first round of clustering, roughly defined groups of genes can be characterized which are differentially regulated across different samples such as different tumor samples or cell lines. This repeated clustering may allow reducing the amount of data and thus improving the signal-to-noise ratio.

It is the attempt of all clustering processes described hereinafter such as the two-way clustering to bring tumors with descriptor profiles such as the same expression profiles in proximity. The resulting dendrogram tells one the conditions which are concentrated into one “pattern”.

The clustering in both steps may be performed by the TIGR MeV software (23) using Euclidian distance and average linkage. The software is used with its default parameters. The identification of groups after the first clustering step and then of signatures after the second clustering step can be performed using SAM (12). The software is used with its default parameters. If a pattern for a set of descriptors has been identified, one can cross-check the accuracy by using alternative software such GENEVESTIGATOR (10, 11).

In this first round, the selection may be rather rough allowing inclusion of groups which are not clearly defined by e.g. visual inspection as the second round of clustering will then sharpen the analysis.

In principle, the accuracy of the analysis will benefit if as many genes and as many samples are analyzed. If, however, e.g. computing power is a limitation, expression may be analyzed of about 100 to about 2000 genes, such as about 200 to about 1000 genes, about 200 to about 800 genes, about 200 to about 600 genes or preferably about 200 to about 400 genes in about 50 to about 400 samples, in about 75 to about 300 samples, in about 100 to about 200 samples and preferably in about 100 samples.

This data is then subjected to a first round of e.g. hierarchical two-way clustering yielding groups of differential regulated genes. These groups of genes are then combined and submitted to a second round of hierarchical two-way clustering. The expression data, which was initially obtained before the first round of clustering, can, of course, be used for the second round of clustering.

This approach allows for more straightforward identification of signatures and thus of discrete disease-specific states. As an example, one may obtain expression data for about 200 to about 400 genes in about 100 RCC samples, which will evenly represent all types of RCCs such as papillary, clear cell and chromophobe RCCs. In the first round of clustering, one may identify 20 groups with overall 100 genes. Group 1 may comprise 10 genes, Group 2 may comprise 20 genes, Group 3 may comprise 6 genes etc.

These 100 genes are then submitted to a second round of hierarchical two-way clustering. The software will then yield three distinguishable patterns, i.e. three signatures for the set of 400 descriptors. As there will be only three signatures for all types of RCCs one knows, that there are three discrete disease-specific states on this level of resolution. In a further step, one can then identify the set of genes for which the expression data most reliably distinguish between the three different states. One can then also analyze how these signatures correlate with e.g. survival rates.

There are further approaches that make identification of groups with differentially regulated genes and thus the identification of signatures and discrete disease-specific states more quickly and which ultimately can help reducing the size of set of descriptors.

This approach looks for analysis of quality and/or quantity of descriptors in known regulatory networks. The identification of groups of e.g. differentially expressed genes within single networks may be more straightforward as some networks may contribute stronger to e.g. tumor development than others. This may allow sorting out of certain networks, reducing the amount of data and thus improving the signal-to-noise ratio.

The invention in a particularly preferred embodiment thus relates to a method of identifying a signature and optionally at least one discrete disease-specific state being implicated in a disease, optionally in a hyper-proliferative disease comprising at least the steps of:

a. Testing for quality and/or quantity of descriptors of genes or gene associated molecules which are associated with at least two regulatory networks in hyper-proliferative disease-specific samples derived from human or animal individuals suffering from said disease or in cell lines of hyper-proliferative diseases;

b. Clustering the results obtained in step a.) comprising at least the steps of:
i. Sorting the results for each descriptor within at least one regulatory network by its quality and/or quantity,
ii. Sorting the disease-specific samples or cell lines for comparable quality and/or quantity of descriptors across all descriptors within one regulatory network;
iii. Identifying different groups of descriptors which are differentially regulated across said disease-specific samples or cell lines within each regulatory network;

c. Combining the descriptors which are identified in step b.)iii.) wherein the quality and/or quantity of said descriptors disease-specific samples or cell lines are already known from step a.);

d. Clustering the results obtained in step c.) comprising at least the steps of:
i. Sorting the results for each descriptor of step c.) by its quality and/or quantity,
ii. Sorting the disease-specific samples or cell lines for comparable quality and/or quantity of descriptors across all descriptors;
iii. Identifying different patterns for the set of descriptors obtained in step c.);
iv. Allocating to each pattern identified in step d.)iii.) a signature;
v. Optionally allocating to each signature identified in step d.),iv.) a discrete disease-specific state.

Again, clustering may be a hierarchical two-way clustering as described above. The clustering in both steps may be performed by the TIGR MeV software (23) using Euclidian distance and average linkage. The software is used with its default parameters. The identification of groups after the first clustering step and then of signatures after the second clustering step can be performed using SAM (12). The software is used with its default parameters. If a pattern for a set of descriptors has been identified, one can cross-check the accuracy by using alternative software such GENEVESTIGATOR (10, 11).

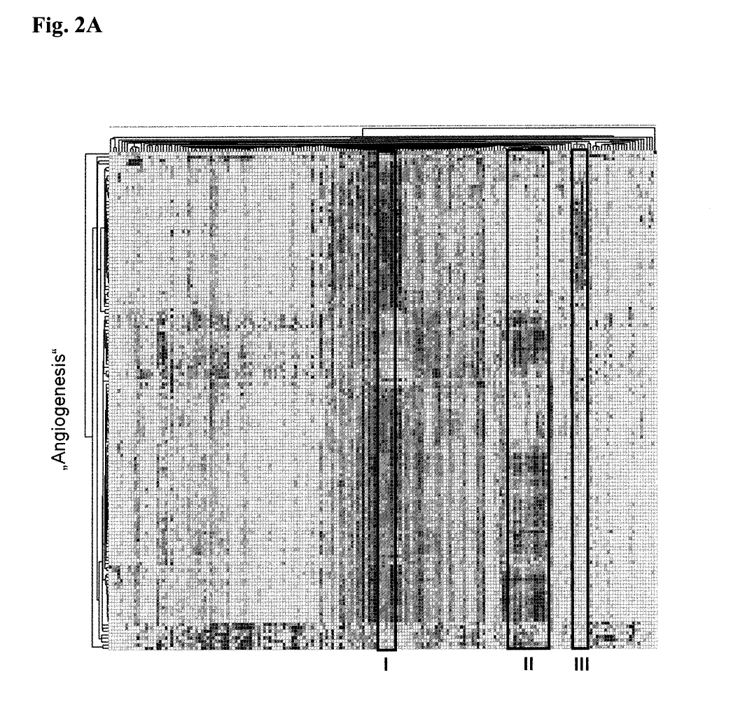

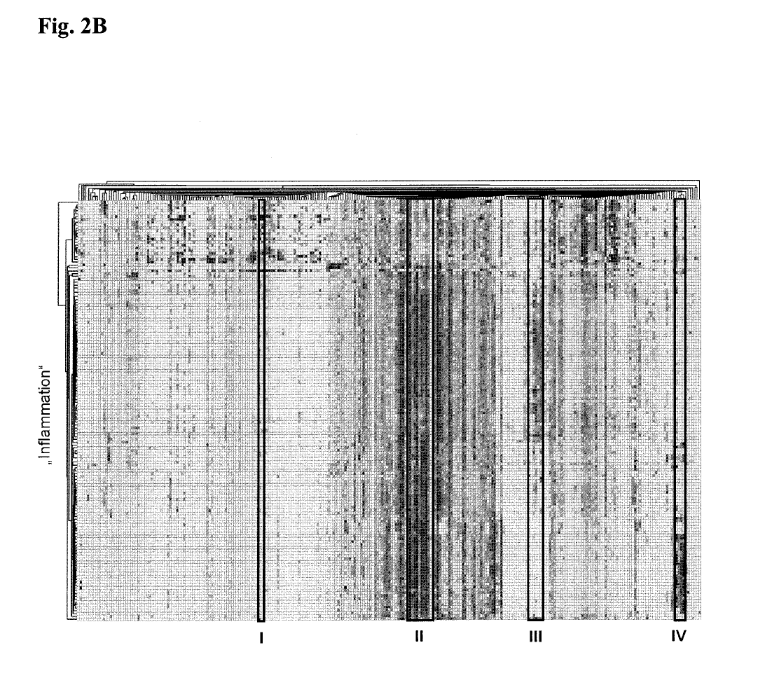

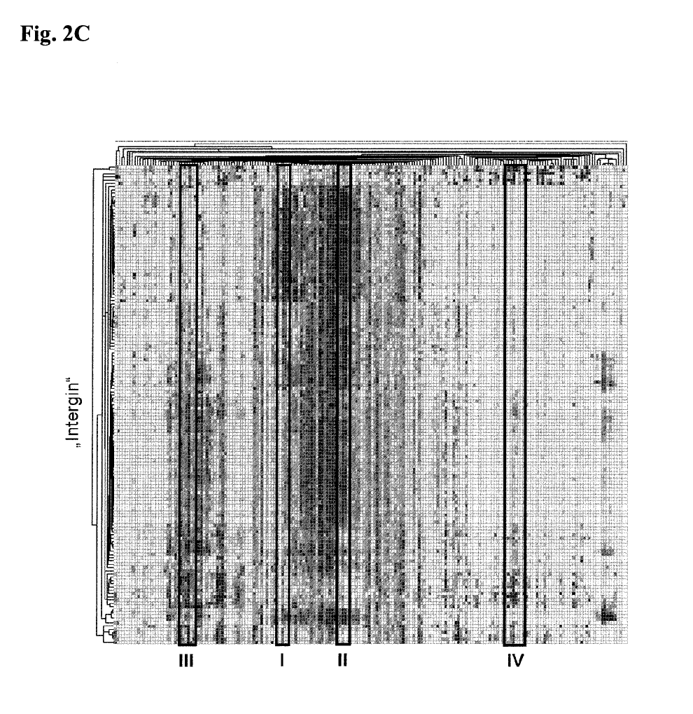

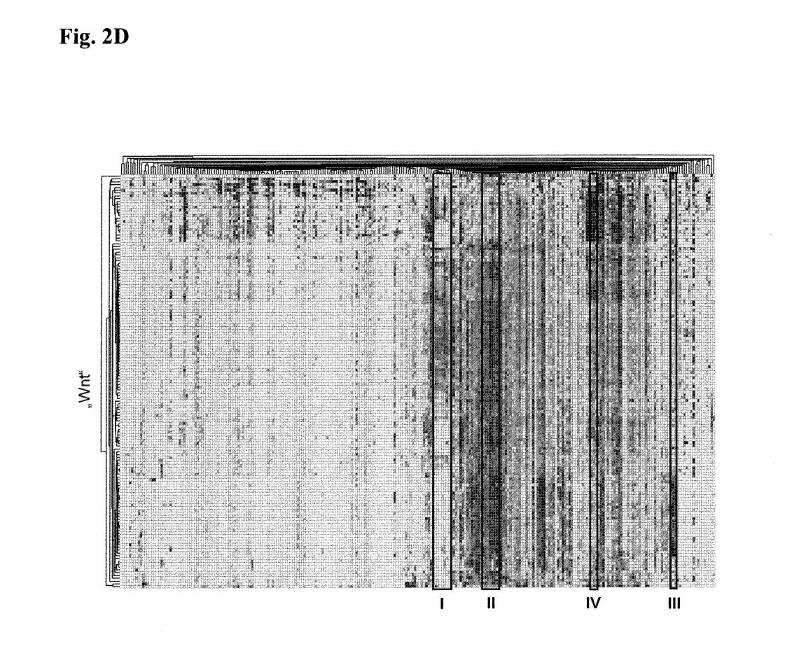

In this embodiment, one will thus run a first clustering round for all genes which are allocated by e.g. a software (see below) to a specific regulatory network (steps a to b)iii.)). This clustering round will be run for different regulatory networks. As a limited set of genes is thus clustered for each network, specific patterns may emerge (see FIG. 2). The descriptors, e.g. the genes of these patterns of all analyzed networks are then combined (step c) and the combined set is subjected to a second clustering (steps d)i. to v.).

One may further streamline this method of identifying signatures and states.

As mentioned, focusing on regulatory networks in a first round of clustering (step b) may be the most reliable way of identifying signatures as a lot of networks will not result in identifiable groups in step b.)iii.). The number of descriptors such as genes which will be combined for the second clustering step will thus be even more reduced.

However, as for the afore-described embodiment with two clustering rounds, a set of descriptors will be obtained which is the combined list of all descriptor groups which were identified in the regulatory network analysis. In the second round of clustering, this set of descriptors will then give rise to patterns, i.e. signatures which allow grouping sample into distinct discrete disease-specific states. In the case of RCC, this approach was taken (see FIGS. 2, 3 and 4) and three states A, B, C were identified.

The networks, which are used in the first clustering round, may be those as they are described in the PANTHER software. In principle, one may use all 165 regulatory networks of the PANTHER software. However, one may incorporate an initial selection step and determine for a given set of samples those regulatory networks which are most affected in the samples. To this end, one may analyze, which networks comprise most frequent descriptors. One may then select the most e.g. 2, 3, 4, 5, 6, 7, 8, 9 or 10 most affected regulatory networks and perform the initial clustering step for these networks only. The example for RCC shows that the general results, i.e. the number of discrete disease-specific states will not differ depending on whether one analyzes the 4 most affected pathways or all 76 affected pathways. Of course, looking at a reduced number of pathways may reduce the number of descriptors, i.e. the set of descriptors, which is used for the second clustering round and may thus improve the signal-to-noise ratio and simplify signature identification.

The analysis may further be simplified by initially identifying descriptors such as genes which are likely affected in a disease. This may be done by e.g. identifying single nucleotide polymorphisms (SNPs) which may be indicative of disease samples. For example and as described in the experimental section, one may analyze samples from disease affected tissue of one individual, where histological analysis confirms that the tissue is affected by the disease, and samples from the same tissue of the same individual, where histological analysis confirms that the tissue is not affected by the disease, for differences in SNPs. These candidate genes can then e.g. be allocated to regulatory networks by e.g. using the PANTHER software. One then identifies the 1, 2, 3, 4, 5 or more regulatory networks which seem most frequently affected because e.g. they comprise the majority of genes for which SNPs were identified. In a subsequent analysis, disease samples are then analyzed for the expression of all genes belonging to these most frequently affected networks even not all of these genes were identified in the SNP analysis. One then uses this expression data in the above described methods.

One may use any initial selection method that yield such candidate descriptors such as methods identifying methylation, phosphorylation etc.

In principle, it could be sufficient to use the above approaches with just e.g. two regulatory networks and analyze just two samples. The reliability and resolution of the analysis will usually be increased if one considers more regulatory networks and tests more samples. Good results may be obtainable by testing e.g. at least about 50 such as about 75, 100, 150 or 200 samples. In terms of regulatory networks, it may be sufficient to analyze the about 3, 4, or 5 networks which seem most affected as may become apparent from e.g. expression data.

It is to be understood that a set of descriptors does not necessarily have to yield different signatures. Thus a chosen set of descriptors may only yield one signature. This will thus indicate that the disease examined has only one discrete disease-specific state. Of course, this assumes that the analysis has been performed with a comprehensive set of sample covering all relevant types of a disease such as samples for clear cell, papillary and chromophobe RCC. The skilled person will know how to select a sufficient number of samples in order to be sure that the majority of all relevant subtypes of a disease have been covered for the analysis.

Of course, a given set of descriptors may also yield multiple signatures such as 2, 3, 4, 5 or more signatures. The number of signatures will indicate the number of discrete disease-specific states that can be observed on this level of resolution for a disease. For example, if one analyzes a comprehensive set of samples for small-cell lung cancer and identifies e.g. three signatures, this means that small cell lung cancer can be characterized by three discrete disease-specific states. If one includes non-small cell lung cancer in the analysis, one may identify two additional signatures, which means that on the level of non-small and small cell lung cancer, these cancers can be classified into five discrete disease-specific states. The selection of the types of samples thus defines on which disease level one may observe discrete disease-specific states.

It is further important to understand that a given signature will unequivocally relate to a discrete disease-specific state. However, a discrete disease-specific state may be described through multiple signatures depending on what type and combination of descriptors have been used for identifying the signatures.

The approaches described above therefore provide just some out of numerous possibilities for identifying signatures and discrete disease-specific states. One may, for example, also use other clustering methods than two-way hierarchical clustering such as Biclustering. These methods have in common that they bring samples of e.g. tumors with similar traits together. The finding of the invention is that these “aligned groups of samples” which may be groups of tumors can then be considered as discrete disease specific states which can be used to characterize a disease.

In general, one can identify groups by grouping samples according to the similarity of a parameter which is attributable to a descriptor (such as expression) over a complete set or over a subset of genes or gene-associated molecules, wherein the similarity is preferably measured using a statistical distance measure such as Euclidian distance, Pearson correlation, Spearman correlation, or Manhattan distance.

However, as the approaches which are mentioned above and which rely e.g. on two-way hierarchical clustering, make use of parameters that are easily accessible and testable on a large scale (e.g. expression on the RNA or protein level), they provide an important tool to identify the number of discrete disease-specific states for a given resolution as well as to identify signatures describing these states.

Once one has identified a number of signatures for a set of descriptors such as by the above-described methods one can further reduce the number of descriptors, which are necessary to distinguish best between different signatures.

To this end, one may analyze samples for which one knows the disease specific states from the above analysis for descriptors that allow the best differentiation of different discrete disease specific states. These descriptors do not necessarily have to be those which led initially to the identification of discrete disease specific stages.

For example, once one has identified discrete specific states for disease-specific samples such as tumor samples by the aforementioned methods making e.g. use of expression data for genes, one may analyze samples for which one knows the discrete disease specific states for expression across all approximately 24.000 genes. One can then select the genes which are most differentially regulated between the samples of different discrete disease specific states and may use these expression patterns as signatures. This sort of analysis may be performed by micro array expression analysis.

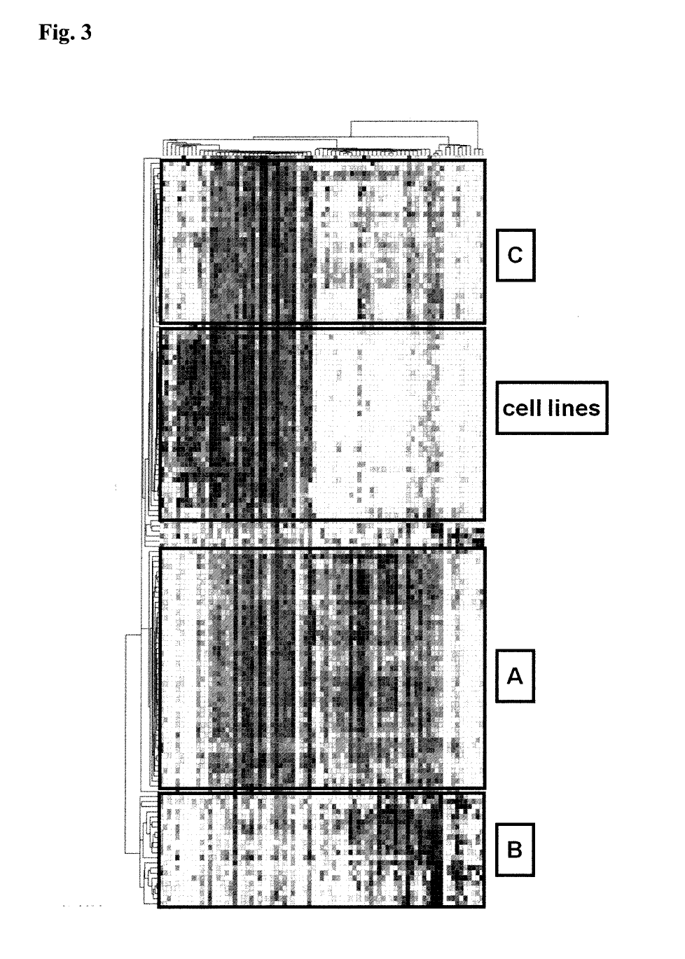

For example, in the examples expression data of 92 genes, i.e. descriptors allowed identification of three signatures and thus of three discrete disease-specific states A, B, and C for RCCs (see FIG. 3). In a further analysis, the samples, for which it was then known whether they are of discrete disease specific state A, B or C, were analyzed for expression of approximately 20.000 genes using the Affymetrix gene chip. The software was then used to identify the genes which are most differentially regulated between sample of discrete disease specific states A, B or C. It turns out that by looking at certain gene lists (see below), one can initially best allocate samples to the discrete RCC specific states B and AC which stands for A and C. The state AC can then be further distinguished into A and C by looking at additional genes.

Using this approach a set of about 50 genes was identified. Overexpression of about 34 of these genes (table 1) and underexpression of about 16 of these genes allows for optimal distinction between state B vs. A and C. Another analysis revealed that overexpression of a group of about 4 (table 3) genes and underexpression of a group of about 19 genes (table 4) is well suited for distinguishing states A and C. These genes do not necessarily are the same as the about 92 genes which originally allowed for identification of the discrete disease specific states.

It is to be understood that the term “genes” in the context of tables 1, 2, 3 and 4 refers to the probes on the Affymetrix gene chip. Tables 1, 2, 3 and 4 all name the Probe Identifiers which allow a clear identification. Where a DNA or amino acid sequence is known for a Probe Identifier is known, this has been indicated. All statements hereinafter which relate to table 1, 2, 3 and 4 preferably only include those genes where the DNA and/or amino acid sequence is known.

In order to identify state B with a reliability of about 50% or more, it is for example sufficient to test for the over- or underexpression of at least one gene of table 1 or 2, respectively. In order to identify state B with a reliability of about 80% or more, it may be sufficient to test for the over- or underexpression of at least two genes of table 1 or 2, respectively. In order to identify state B with a reliability of about 90% or more, it may be sufficient to test for the over- or underexpression of at least three genes of table 1 or 2, respectively. In order to identify state B with a reliability of about 95% or more, it may be sufficient to test for the over- or underexpression of at least five genes of table 1 or 2, respectively. In order to identify state B with a reliability of about 99% or more, it may be sufficient to test for the over- or underexpression of at least six genes of table 1 or 2, respectively.

In order to identify state A vs. C with a reliability of about 50% or more, it is for example sufficient to test for the over- or underexpression of at least two genes of table 3 or 4, respectively. In order to identify state A vs. C with a reliability of about 70% or more, it may be sufficient to test for the over- or underexpression of at least three genes of table 3 or 4, respectively. In order to identify state A vs. C with a reliability of about 80% or more, it may be sufficient to test for the over- or underexpression of at least four genes of table 3 or 4, respectively. In order to identify state A vs. C with a reliability of about 90% or more, it may be sufficient to test for the over- or underexpression of at least five genes of table 3 or 4, respectively. In order to identify state A vs. C with a reliability of about 95% or more, it may be sufficient to test for the over- or underexpression of at least six genes of table 3 or 4, respectively. In order to identify state A vs. C with a reliability of about 99% or more, it may be sufficient to test for the over- or underexpression of at least seven genes of table 3 or 4, respectively.

In order to identify a set of descriptors, which allows best distinguishing different signatures and thus discrete states, one can use the SAM software (12) and set an at least a 2-fold change in the expression level as a selection parameter. If one wants to increase the preciseness of the signatures and at the same time to reduce the number of descriptors which is used to differentiate between different signature, one can the threshold higher such as 3, 4, 5 or more.

It is to be noted that the invention wherever it mentions methods of identifying discrete disease-specific states, signatures etc. always considers that the quality and/or quantity of descriptors has to be tested. This testing may include technical means such as use of e.g. micro-arrays to determine expression of genes. If the invention considers applying such methods by relying on and using data which are indicative of the quality and/or quantity of descriptors and which are deposited in e.g. databases after they have been determined using technical means, these methods will be run on technical devices such as a computer. All methods as they are described herein for identifying discrete disease-specific states, signatures etc. may therefore be performed in a computer-implemented way.

As will become apparent from the examples, the discrete disease-specific states, which were identified for RCCs can also be found to some extent in other hyper-proliferative diseases.

The aforementioned methods are thus suitable to identify a comprehensive set of signatures and thus discrete disease-specific states within a set of samples such as patient samples for hyper-proliferative diseases or cell lines of hyper-proliferative diseases. The signature and states can then be correlated to clinically relevant parameters such as average survival time and thus allow a clinically important characterization of diseases by easily accessible parameters such as expression data. It is, however, new that such signatures do not necessarily correlate with phenotypic histological characterization of the respective disease but rather seem to describe discrete states on e.g. the molecular level that characterize the disease development.

As pointed out above, these discrete disease-specific states allow obviously for some change (e.g. mutations, de-regulation etc.) until a threshold level is reached and switching to another discrete disease-specific state occurs.

It is currently not clear whether e.g. the three states of RCCs represent consecutive states such that first state A occurs which switches then to state B and then to state C or whether these states occur in parallel or are a combination of consecutives and parallel development. The important aspect, however, is that e.g. hyper-proliferative diseases such as RCCs occur in discrete states which can be linked to clinically relevant parameters such as survival time. One can for example test whether chemical compounds are capable of switching cell from a state being correlated with short survival time to a state being correlated to long survival time. This will be explained in more detail below.

Further, the signatures and states, which were found to characterize a disease, can be used to characterize other diseases. This, for example may allow predicting the efficacy of a pharmaceutically active compound for different disease if these diseases can be characterized by the same states.

In the following, we will set forth in detail that signatures and discrete disease-specific states can be used for diagnostic, prognostic, analytical and therapeutic purposes. These aspects will be discussed in parallel for discrete disease-specific states and signatures as if these terms were interchangeable. It has, however, to be born in mind that a discrete disease-specific state can be described through various signatures depending on the type and combinations of descriptors chosen. If in the following the term signature is used this is thus meant to incorporate all signatures that can be used to describe a single discrete disease-specific state. Further, all embodiments, which are discussed for signatures, equally apply to discrete disease-specific states.

The invention as mentioned relates to discrete disease-specific states for use as a diagnostic and/or prognostic marker in classifying samples from patients, which are suspected of being afflicted by a disease, optionally by a hyper-proliferative disease. The invention also relates to discrete disease-specific states for use as a diagnostic and/or prognostic marker in classifying cell lines of a disease, optionally of a hyper-proliferative disease. The invention further relates to discrete disease-specific states for use as a target for development of pharmaceutically active compounds.

The invention also relates to signatures for use as a diagnostic and/or prognostic marker in classifying samples from patients, which are suspected of being afflicted by a disease, optionally by hyper-proliferative disease wherein the signature comprises a qualitative and/or quantitative pattern of at least one descriptor and wherein the signature is indicative of a discrete disease-specific state. As for states, the invention also relates to signatures for use as a diagnostic and/or prognostic marker in classifying cell lines of a disease, optionally of a hyper-proliferative disease wherein the signature comprises a qualitative and/or quantitative pattern of at least one descriptor and wherein the signature is indicative of a discrete disease-specific state. Further, the invention relates to signatures for use as a read out for a target in the development, identification and/or application of pharmaceutically active compounds, wherein the signature comprises a qualitative and/or quantitative pattern of at least one descriptor and wherein the signature is indicative of a discrete disease-specific state. The target may be the discrete disease specific state which is reflected by the signature.

As mentioned above, a discrete disease specific state can be described by way of one or more signatures comprising at least two descriptors, which have been identified by comparing at least two regulatory networks in at least two patient derived-samples or cell lines.

The discrete disease-specific states and signatures relating thereto can be used for diagnostic purposes. Thus, samples of patients suffering from a disease such as a hyper-proliferative disease may be analyzed for their discrete disease-specific states and classified accordingly. The importance of discrete disease-specific states for classifying samples and thus for diagnosing patients become clear from the experiments on RCCs.

These examples show that it may be more informative to differentiate RCCs based on their discrete disease-specific state than by their phenotypic classification such as papillary, clear cell and chromophobe RCCs. In fact, the experiments show that papillary RCC samples, which were derived from different patients, may differ with respect to their discrete disease specific states. At the same time different papillary and clear cell RCCs may be characterized by the same discrete disease-specific state.

Thus, even though tumors may look comparable on the histological level, they may differ in terms of the underlying molecular mechanisms. Conversely, tumors may show different histological properties but still share the same underlying molecular mechanism in term of a discrete disease specific state. Given that the three discrete disease specific states, which could be identified for RCCs, clearly correlate with average survival time, classifying samples not e.g. according to their histological properties but according to their discrete disease-specific molecular state provides a new important classification scheme. Further, the knowledge about discrete disease specific states can help to diagnose ongoing disease development in samples obtained from patients early on at a point in time where histological changes or other phenotypic properties are not discernible yet.

The present invention in one aspect thus relates to a method of diagnosing, stratifying and/or screening a disease, optionally a hyper-proliferative disease in at least one patient, which is suspected of being afflicted by a disease, optionally by a hyper-proliferative disease or in at least one cell line of a disease, optionally of a hyper-proliferative disease comprising at least the steps of:

a. Providing a sample of a human or animal individual which is suspected of being afflicted by said disease;

b. Testing said sample for a signature;

c. Allocating a discrete disease-specific state to said sample based on the signature determined in step b.).

The sample may be a tumor sample.

There may be different ways to test for a signature. If the signature is not known yet, one may identify it as described above. If the signature is already known, one can test for it by analyzing the quality and/or quantity of descriptors that were used for identification of the signature. One can also use optimized signatures which allow best differentiation between different states. If for example the signature is based on expression data for a set of given genes or gene-associated molecules such as RNAs or proteins, one can test for a signature by simply determining the expression pattern for this set of molecules. This may be done by standard methods such as by micro-array expression analysis.

If one has identified the signature, one also knows the discrete disease specific state which correlates with this signature. Using such methods one can thus classify patient samples by common molecular mechanisms that lead to the same discrete disease specific molecular states. If such discrete disease specific states occur before phenotypic changes become apparent, it is thus possible to diagnose a hyper-proliferative disease such as RCC early on.

Preferably, “discrete disease specific states, substates or levels” are used as a new stratifying tool for categorizing diseases which otherwise are diagnosed on a general level.

Thus, the invention preferably relates in one embodiment to identifying discrete disease specific states, substates, levels, etc. by analyzing disease such as hyper-proliferative disease for signatures being indicative of discrete disease specific states, substates and levels as described above. This analysis will be performed for a specific type of hyper-proliferative disease such as e.g. RCC, lung cancer, breast cancer etc. Thus, the diseases may be identified by common selection criteria such as the organs being affected. However, initially no attention will be given to sub-classifications of these hyper-proliferative diseases, which are based on e.g. histological classification schemes. Once one has identified different discrete disease specific states for a disease like e.g. RCCs, one can test samples as described above for ongoing disease development already at a point in time when no phenotypic changes are recognizable. The discrete disease specific state therefore usually allows one to directly predict which sub-type of the disease in question is developing (e.g. state A, B, or C for RCC). These subtypes are correlated with e.g. clinically relevant parameters such as survival time. Thus, the term discrete disease specific state, substate or level preferably allows distinguishing different subtypes of a disease according to a new classification scheme, which links the subtype to clinically or pharmacologically important parameters. The finding of the present invention that discrete disease specific states exist in diseases and can be correlated with subtypes that are characterized not necessarily by their histological properties but by clinically or pharmacologically relevant parameters thus allows deciphering disease through a new code which is based on the discrete disease specific states, substates and levels.

The knowledge that discrete disease-specific states exist e.g. in RCC and other hyper-proliferative diseases can also be used to stratify patient cohorts undergoing clinical trials for new treatments of RCC or other hyper-proliferative diseases. As mentioned herein, certain pharmaceutically active agents may act only on specific discrete disease-specific states. If a patient cohort which undergoes a clinical trial with such an active agent consists mainly of individuals with other discrete disease-specific states, any effects of the pharmaceutically active agent on the specific discrete disease-specific state may not be discernible. Such effects may become, however, statistically significant if the patient cohort is grouped according to the discrete disease-specific states. Thus, the knowledge on the existence of discrete disease-specific states can be used to stratify test populations undergoing clinical trials according to their discrete disease-specific states.

Further, once a discrete disease specific state is known, the knowledge about its existence can be used to test whether it also occurs as a subtype in different hyper-proliferative diseases. The discrete disease specific states, substates and levels and the signature relating thereto can thus be used to screen different diseases for the presence of these subtypes.

The classification of samples for their discrete disease specific states through identifying respective signatures can thus be used for diagnosing disease such as hyper-proliferative diseases. However, the classification of samples, be it of patients or cell lines for diseases such as hyper-proliferative diseases, for their discrete disease specific states has further implications.

Given that discrete disease specific states seem to reflect decisive stages of the underlying molecular disease mechanisms, they can be linked to relevant clinical and pharmacological parameters such as average survival times or responsiveness to drugs. This means that analyzing samples of patients for their respective discrete disease specific molecular states does not only allow diagnosing the type of the disease at an early point in time but also makes a prognosis possible as to the future course of the disease. Thus, one will early know whether a patient suffers from e.g. RCC and whether this RCC will be an aggressive or comparatively moderate form. This prognosis can then be used for therapeutic purposes when making decisions as to the kind of medication, physical treatment or surgery.

Further, the possibility of assigning a discrete disease specific state to samples allows analyzing the effectiveness of treatments with specific drugs. For example, one can test a patient or a population of patients suffering from a hyper-proliferative disease for (i) their reaction towards treatment with a pharmaceutically active agent and (ii) for their discrete disease specific molecular state. The reaction towards treatment may be measured by e.g. the quality of and quantity of clinical improvement. One can then try to correlate such responders towards treatment with discrete disease specific states. If it turns out that patients for which the disease is characterized by a specific discrete disease specific state react more favorably towards treatment, these patients show a higher responsiveness towards treatment.

The invention in one aspect thus relates to a method of determining the responsiveness of at least one human or animal individual which is suspected of being afflicted by a disease, optionally by a hyper-proliferative disease towards a pharmaceutically active agent comprising at least the steps of:

a. Providing a sample of at least one human or animal individual which is suspected of being afflicted by a disease before the pharmaceutically active agent is administered;

b. Testing said sample for a signature;

c. Allocating a discrete disease-specific state to said sample based on the signature determined;

d. Determining the effect of a pharmaceutically active compound on the disease symptoms and/or the discrete-disease specific state in said individual;

e. Identifying a correlation between the effects on disease symptoms and/or the discrete disease-specific state and the initial discrete disease-specific state of the sample.

The signature may be tested for as described above. The sample may be a tumor sample.

Being able to predict the responsiveness of e.g. patients with a discrete disease specific state towards treatment is helpful in many aspects. For example, if such responsiveness is known, one can pre-select patients for treatment. Identification of signatures and discrete disease specific states can thus serve as companion diagnostics, which allow pre-selecting patients for effective treatment. Tools for identifying patients that will respond to a particular treatment become more and more important with public health systems requiring such tests in order to reimburse expensive therapies. Being able to predict whether a specific group of patients which is characterized by their discrete disease specific states will react favorably towards a specific pharmaceutically active agent is also important for other areas. For example, a lot of drugs receive their initial marketing authorization from regulatory agencies such as the FDA for a specific indication only. Frequently, one then tries to test whether such drugs are also effective for treating other diseases. Such clinical trials are, however, extremely costly.

If one knew upfront that only patients with a specific discrete disease specific state have reacted positively towards a specific drug and if one now tests this drug for other diseases, one will be able to conduct such clinical trials with a significantly smaller patient group by selecting only patients with the discrete disease specific profile which has shown a positive response when patients with the same state were tested albeit for a different disease. These clinical trials will not only be less costly in view of the smaller test population, they are also likely to lead to a positive outcome as the effects of the treatment may be more pronounced and thus more easily discernible by statistical methods as the signal-to-noise ratio will be improved.

Being able to predict the responsiveness of a treatment also forms part of the prognostic aspects of the invention.

The invention in one embodiment thus relates to a method of predicting the responsiveness of at least one patient which is suspected of being afflicted by a disease, optionally by a hyper-proliferative disease towards a pharmaceutically active agent comprising at least the steps of:

a. Determining whether a correlation exists between effects on disease symptoms and/or discrete disease-specific states and the initial discrete disease-specific states as a consequence of administration of a pharmaceutically active agent as described above;

b. Testing a sample of a human or animal individual which is suspected of being afflicted by a disease, optionally by a hyper-proliferative disease for a signature;

c. Allocating a discrete disease-specific state to said sample based on the signature determined;

d. Comparing the discrete disease-specific state of the sample in step c. vs. the discrete disease-specific state for which a correlation has been determined in step a.);

e. Predicting the effect of a pharmaceutically active compound on the disease symptoms in said patient.

The sample may be a tumor sample.

The finding that diseases such as hyper-proliferative diseases are characterized by discrete disease specific states also allows new approaches for development and/or identification of new therapeutically active agents.

As mentioned above, samples from patients can be characterized as to their discrete disease specific states. Further, cell lines of diseases may also display such discrete disease specific states. It is assumed that a pharmaceutically active agent towards which a patient with a discrete disease specific state is responsive may in some instances induce a switch to another discrete disease specific sate. This other discrete disease specific state may either be a completely new discrete disease specific state or it may be a discrete disease specific state, which has been found in other patients. For example, a pharmaceutically active agent may induce a switch from a discrete disease specific state which is correlated with low average survival times to a discrete disease specific state which is correlated with a longer average survival time. The discrete disease specific states and signatures relating thereto may be identified as described above.

If indeed a pharmaceutically active agent is capable of inducing a switch of discrete disease specific states, one can use discrete disease specific states and the signatures relating thereto as a read-out parameter for the potential effectiveness of pharmaceutically active agents. The target on which the pharmaceutically active agent would act is thus the discrete disease specific state. The discrete disease specific states are thus considered to targets of pharmaceutically active agents.

The invention in one embodiment therefore relates to a method of determining the effects of a pharmaceutically active compound, comprising at least the steps of:

a. Providing a sample of at least one human or animal individual which is suspected of being afflicted by a disease, optionally by a hyper-proliferative disease or a cell line of a disease, optionally of a hyper-proliferative disease before a pharmaceutically active agent is applied;

b. Testing said sample or cell line for a signature;

c. Allocating a discrete disease-specific state to said sample or cell line based on the signature determined;

d. Testing said sample or cell line for a signature after the pharmaceutically active agent is applied;

e. Allocating a discrete disease-specific state to said sample or cell line based on the signature determined;

f. Comparing the discrete disease-specific states identified in steps c.) and e.).

The sample may be a tumor sample.

The effects that are determined by this method may e.g. allow identification of compounds which may have a positive influence on the disease if e.g. a switch to a discrete disease specific state correlated with a more favorable clinical parameter such as increased survival time is observed. The methods may, however, also allow identification of toxic compounds if these compounds induce a switch to a discrete disease specific state correlated with a less favorable clinical parameter such as decreased survival time. These methods may thus be used as assays in the development, identification and/or screening of potential pharmaceutically active compounds, e.g. to determine the potential effectiveness of a pharmaceutically active compound in a disease such as a hyper-proliferative disease. These assays may also be used for determining the toxicity of a pharmaceutically active compound.

Such discrete state-related assay systems for active and/or toxic drug candidates could be of enormous value to identify new pharmaceuticals. With the reasonable assumption that certain discrete states of a tumor are not just indicative for the status of being a hyper-proliferating cell but also being related e.g. to the aggressiveness of a tumor or survival time of a patient, the switch in state monitored by switch in signature marks an interesting screening system as a general “read out” for changing a tumor status. So the “read out” is related to functional efficacy rather than blocking a certain molecular target not necessarily being related to tumor function. Such screening system would simply pick up any compound switching the state irrespective of the molecular target of interaction. Such screening resembles assays interfering with virus propagation in cell cultures rather than screening for inhibitors of a certain viral enzyme just as reverse transcriptase.

On the other hand such assays could be indicative for the tumorgenicity of compounds turning a status characteristic for a healthy cell into a status characteristic for the status of a hyperproliferative cell.

For example, expression analysis has been performed for HS 294T cells. After administration of 5 mM acetyl cysteine at 6 hours, expression analysis revealed presence of a discrete disease specific state corresponding to state B of RCCs. This state could not be detected before administration. This indicates that acetyl cystein may induce a switch to this state B in the HS 294T cells.

Similarly, expression analysis has been performed for human malignant peripheral nerve sheath tumor (90-8) cells. These cells were infected with G207, an ICP34.5-deleted oncolytic herpes simplex virus (oHSV). After infection, expression analysis revealed presence of a discrete disease specific state corresponding to state B of RCCs. This state could not be detected before infection. This indicates that oHSV may induce a switch to this state B in the 90-8 cells.

Compounds such as mevalonate, UO126, MK886, deferoxamine, paclitaxel may have similar effects.

The finding of discrete disease specific states as being characteristic for diseases thus allows for various diagnostic, prognostic, therapeutic, screening and developmental approaches. In most of these approaches, one uses signatures as a read-out parameter for the presence of a discrete disease specific state. Of course, one will aim to use signatures, which can be easily and reliably be determined.

Therefore, one may preferably use signatures, wherein genes or gene-associated molecules such as RNA and proteins are used as descriptors and wherein the expression pattern thereof serves as a signature. The advantage of this approach is that one can rely on common micro-array expression profiling for identifying signatures. Further, one can use existing expression data from micro-array analysis for identifying relevant signatures and states by making use of the aforementioned identification methods.

As mentioned hereinafter, three different discrete disease specific states have been identified for RCC. Further, these states were found at least to some degree in other hyper-proliferative diseases such as ovarian carcinoma. These states can be described by signatures, which are based on expression data.

As this data provides a reliable and straightforward read-out, the present invention relates in one embodiment to a signature for use as diagnostic and/or prognostic marker in the classification of a disease such as a hyper-proliferative disease, preferably of cancers, more preferably of renal cell carcinoma, or for use as read out of a target for developing, identifying and/or screening of a pharmaceutically active compound, wherein the signature is characterized by:

a. an overexpression of at least one gene of table 1, and/or

b. an underexpression of at least one gene of table 2.

The presence of this signature will be indicative of a discrete disease-specific state at least in RCC, which is indicative of an intermediate average survival time where about 45 to about 55% such as about 50% of patients can be expected to live after 60 months. Preferably, the presence of this signature will be indicative of a discrete disease-specific state at least in RCC, which is indicative of an intermediate average survival time where about 40 to about 50% such as about 45% of patients can be expected to live after 90 months.

Such a signature is characterized by:

a. an overexpression of at least one gene of table 1, and/or

b. an underexpression of at least one gene of table 2, and wherein determination of the over- and/or underexpression of at least one gene of table 1 and table 2 respectively allows assigning a discrete disease-specific state with a likelihood of ≧50%.

Such a signature is characterized by:

a. an overexpression of at least one gene of table 1, and/or

b. an underexpression of at least one gene table 2, and wherein determination of the over- and/or underexpression of at least two genes of table 1 and table 2 respectively allows assigning a discrete disease-specific state with a likelihood of ≧80%.

Such a signature is characterized by:

a. an overexpression of at least one gene of table 1, and/or

b. an underexpression of at least one gene table 2, and wherein determination of the over- and/or underexpression of at least three genes of table 1 and table 2 respectively allows assigning a discrete disease-specific state with a likelihood of ≧90%.

Such a signature is characterized by:

a. an overexpression of at least one gene of table 1, and/or

b. an underexpression of at least one gene table 2, and wherein determination of the over- and/or underexpression of at least four genes of table 1 and table 2 respectively allows assigning a discrete disease-specific state with a likelihood of ≧95%.

Such a signature is characterized by:

a. an overexpression of at least one gene of table 1, and/or

b. an underexpression of at least one gene table 2, and wherein determination of the over- and/or underexpression of at least five genes of table 1 and table 2 respectively allows assigning a discrete disease-specific state with a likelihood of ≧99%.

It is to be understood that even though analysis of a single gene of table 1 or table 2 may be sufficient for assigning the discrete disease-specific state, the likelihood of a correct assignment will increase if more genes are analyzed. Thus, the signature also includes analysis for the overexpression of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 genes of table 1 and/or the underexpression of at least 2, 3, 4, 5, 6, 7, 8, 9, 10, 11, 12, 13, 14, 15 or 16 genes of table 2. It may be most straightforward to look at the expression data of all genes of table 1 and/or table 2. By considering more than just one descriptor may allow to determine a signature and thus a discrete disease with a likelihood of at least about 50%, at least about 60%, at least about 70%, at least about 80%, at least about 90%, at least about 95%, at least about 98% or at least about 99%. Preferably the signatures are determined through analyzing 2, 3, 4, 5, 6, 7, 8, 9, or 10 genes of table 1 and/or table 2.

The present invention relates in one embodiment to a signature for use as diagnostic and/or prognostic marker in the classification of hyper-proliferative diseases such as cancers, preferably of renal cell carcinoma, or for use as target for development of pharmaceutically active compounds, wherein the signature is characterized by:

a. an overexpression of at least one gene of table 3, and/or

b. an underexpression of at least one gene of table 4.

The presence of this signature will be indicative of a discrete disease-specific state at least in RCC, which is indicative of a low average survival time where e.g. about 30% to about 45% such as about 40% of patients can be expected to live after 60 months. Preferably, the presence of this signature will be indicative of a discrete disease-specific state at least in RCC, which is indicative of an intermediate average survival time where about 5 to about 30% such as about 10% to 20% of patients can be expected to live after 90 months.

Such a signature is characterized by:

a. an overexpression of at least one gene of table 3, and/or

b. an underexpression of at least one gene of table 4, and wherein determination of the over- and/or underexpression of at least two genes of table 3 and table 4, respectively allows assigning a discrete disease-specific state with a likelihood of ≧50%.

Such a signature is characterized by:

a. an overexpression of at least one gene of table 3, and/or

b. an underexpression of at least one gene table 4, and wherein determination of the over- and/or underexpression of at least three genes of table 3 and table 4 respectively allows assigning a discrete disease-specific state with a likelihood of ≧70%.

Such a signature is characterized by:

a. an overexpression of at least one gene of table 3, and/or

b. an underexpression of at least one gene table 4, and wherein determination of the over- and/or underexpression of at least four genes of table 3 and table 4 respectively allows assigning a discrete disease-specific state with a likelihood of ≧80%.

Such a signature is characterized by:

a. an overexpression of at least one gene of table 3, and/or

b. an underexpression of at least one gene table 4, and wherein determination of the over- and/or underexpression of at least five genes of table 3 and table 4 respectively allows assigning a discrete disease-specific state with a likelihood of ≧90%.

Such a signature is characterized by:

a. an overexpression of at least one gene of table 3, and/or

b. an underexpression of at least one gene table 4, and wherein determination of the over- and/or underexpression of at least six genes of table 3 and table 4 respectively allows assigning a discrete disease-specific state with a likelihood of ≧95%.

Such a signature is characterized by:

a. an overexpression of at least one gene of table 3, and/or

b. an underexpression of at least one gene table 4, and wherein determination of the over- and/or underexpression of at least seven genes of table 3 and table 4 respectively allows assigning a discrete disease-specific state with a likelihood of ≧99%.

It is to be understood that even though analysis of a single gene of table 3 or table 4 may be sufficient for assigning the discrete disease-specific state the likelihood of a correct assignment will increase if more genes are analyzed. Thus, the signature also includes analysis for the overexpression of at least 2, 3, or 4 genes of table 3 and/or the underexpression of at least 2, 3, 4, 5, 6, 7, 8, 9, 10, 11, 12, 13, 14, 15, 16, 17, 18 or 19 genes of table 4. It may be most straightforward to look at the expression data of all genes of table 3 and/or table 4. By considering more than just one descriptor may allow to determine a signature and thus a discrete disease with a likelihood of at least about 50%, at least about 60%, at least about 70%, at least about 80%, at least about 90%, at least about 95%, at least about 98% or at least about 99%. Preferably the signatures are determined through analyzing 2, 3, 4, 5, 6, 7, 8, 9, or 10 genes of table 3 and/or table 4.

The present invention relates in one embodiment to a signature for use as diagnostic and/or prognostic marker in the classification of hyper-proliferative diseases such as cancers, preferably of renal cell carcinoma, or for use as target for development of pharmaceutically active compounds, wherein the signature is characterized by:

a. an underexpression of at least one gene of table 3, and/or

b. an overexpression of at least one gene of table 4.

The presence of this signature will be indicative of a discrete disease-specific state at least in RCC, which is indicative of a high average survival time where about 70 to about 90% such as about 80% of patients can be expected to live after 60 months. Preferably, the presence of this signature will be indicative of a discrete disease-specific state at least in RCC, which is indicative of an intermediate average survival time where about 60 to about 80% such as about 70% of patients can be expected to live after 90 months.

Such a signature is characterized by:

a. an underexpression of at least one gene of table 3, and/or

b. an overexpression of at least one gene of table 4, and wherein determination of the under- and/or overexpression of at least two genes of table 3 and table 4, respectively allows assigning a discrete disease-specific state with a likelihood of ≧50%.

Such a signature is characterized by:

a. an underexpression of at least one gene of table 3, and/or

b. an overexpression of at least one gene table 4, and wherein determination of the under- and/or overexpression of at least three genes of table 3 and table 4 respectively allows assigning a discrete disease-specific state with a likelihood of ≧70%.

Such a signature is characterized by:

a. an underexpression of at least one gene of table 3, and/or

b. an overexpression of at least one gene table 4, and wherein determination of the under- and/or overexpression of at least four genes of table 3 and table 4 respectively allows assigning a discrete disease-specific state with a likelihood of ≧80%.

Such a signature is characterized by:

a. an underexpression of at least one gene of table 3, and/or

b. an overexpression of at least one gene table 4, and wherein determination of the under- and/or overexpression of at least five genes of table 3 and table 4 respectively allows assigning a discrete disease-specific state with a likelihood of ≧90%.

Such a signature is characterized by:

a. an underexpression of at least one gene of table 3, and/or

b. an overexpression of at least one gene table 4, and wherein determination of the under- and/or overexpression of at least six genes of table 3 and table 4 respectively allows assigning a discrete disease-specific state with a likelihood of ≧95%.

Such a signature is characterized by:

a. an underexpression of at least one gene of table 3, and/or

b. an overexpression of at least one gene table 4, and wherein determination of the under- and/or overexpression of at least seven genes of table 3 and table 4 respectively allows assigning a discrete disease-specific state with a likelihood of ≧99%.

It is to be understood that even though analysis of a single gene of table 3 or table 4 may be sufficient for assigning the discrete disease-specific state the likelihood of a correct assignment will increase if more genes are analyzed. Thus, the signature also includes analysis for the underexpression of at least 2, 3 or 4 genes of table 3 and/or the overexpression of at least 2, 3, 4, 5, 6, 7, 8, 9, 10, 11, 12, 13, 14, 15, 16, 17, 18 or 19 genes of table 4. It may be most straightforward to look at the expression data of all genes of table 3 and/or table 4. By considering more than just one descriptor may allow to determine a signature and thus a discrete disease with a likelihood of at least about 50%, at least about 60%, at least about 70%, at least about 80%, at least about 90%, at least about 95%, at least about 98% or at least about 99%. Preferably the signatures are determined through analyzing 2, 3, 4, 5, 6, 7, 8, 9, or 10 genes of table 3 and/or table 4.

These signatures and the discrete disease specific states relating thereto can preferably be used for the aforementioned diagnostic, therapeutic and prognostic purposes in the context of RCC. However, as these signatures and states were also identified in bladder cancer, breast cancer, ovarian cancer, myeloma, colorectal cancer, large cell lymphoma, oral squamous cell carcinoma, cervical squamous cell carcinoma, thyroid cancer, adenocarcinoma, they may also be used for the above purposes in the context of these cancer types.

Signature of high, intermediate and low survival time as mentioned above (e.g. about 80% after 90 months) may be determined for RCCs as well as the preceding cancers by analyzing the expression of the genes CD34 (SEQ ID Nos.: 780 (DNA sequence), 781 (amino acid sequence)), DEK (SEQ ID Nos.: 782 (DNA sequence), 783 (amino acid sequence)) and MSH 6 (SEQ ID Nos.: 784 (DNA sequence), 785 (amino acid sequence)).

A discrete state with high survival time can be identified by high expression of CD34, low to high expression of DEK and low to high expression of MSH6. A discrete state with intermediate survival time can be identified by low to high expression of CD34, no expression of DEK and no expression of MSH6. A discrete state with low survival time can be identified by low expression of CD34, low to high expression of DEK and low to high expression of MSH6. These signatures may be used in all embodiments as described herein.

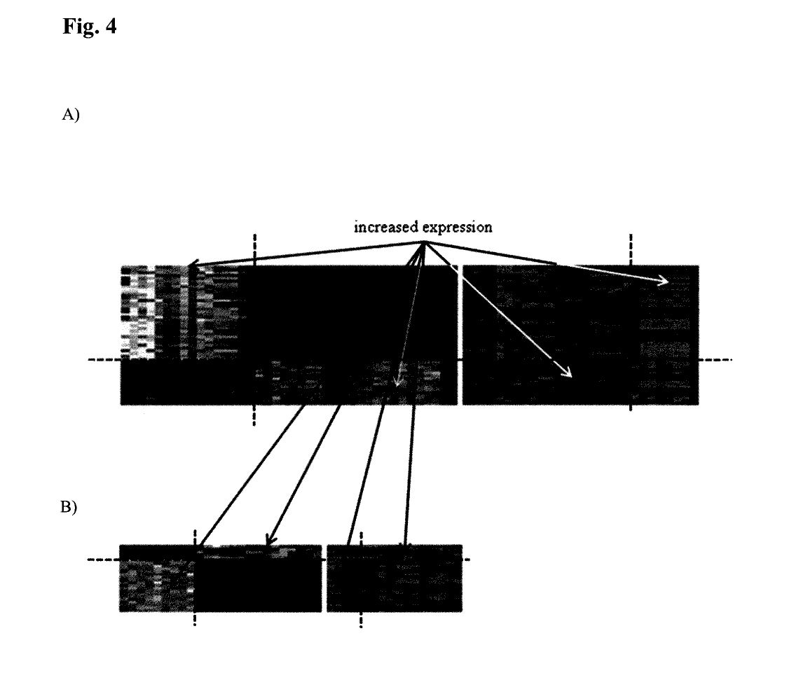

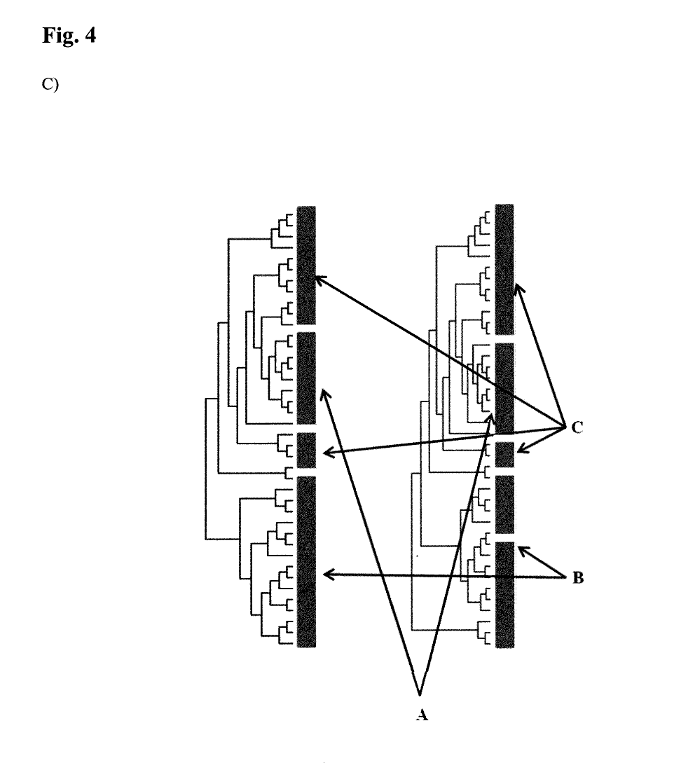

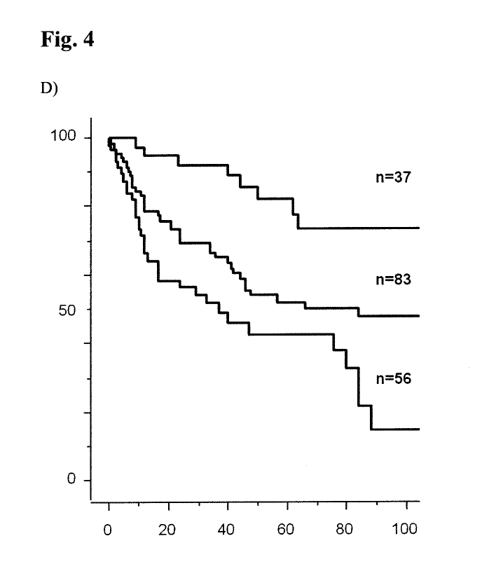

As already mentioned above, the present invention hinges on the finding that hyper-proliferative diseases such as renal cell carcinoma seem to exist in different discrete disease-specific states. Three such discrete disease-specific states have originally been identified for renal cell carcinoma (RCC) using a two times, two-way hierarchical clustering approach. In the first step, differentially expressed genes within a distinct tumor cohort, which are however commonly deregulated for some but not for all tumors of this cohort, were identified (see FIGS. 2A to 2D). In a second step, these genes enabling a differentiation between tumor sub-groups were picked and combined into a matrix for the second two-way hierarchical clustering step against the same tumor cohort. For the case of RCC, this revealed three discrete disease specific states which were labeled A, B, and C 8 (see FIG. 3). Some of these states were identified in other tumors (see FIG. 4). For RCC, certain genes were identified as being suitable descriptors (see above and e.g. Tables 1 to 4). The expression profile of these genes yields different signatures indicative of the three afore-mentioned states.

With this knowledge at hand, computer-implemented, algorithm based approaches were undertaken to identify further sets of genes which allow characterization of RCC by its three discrete disease-specific states.

These computer-implemented, algorithm based approaches which are described in the following led to the identification of approximately 454 genes depicted in Table 10, the expression patterns of which can be used to distinguish between the discrete RCC specific states B vs AC. The expression pattern of another set of approximately 195 genes which are depicted in Table 11 can be used to distinguish between the discrete RCC specific states A vs C. In the following, the implications of these results are set forth. Then, the computer-implemented, algorithm based approaches are explained in further detail.

As mentioned, the expression pattern of about 454 genes, which are listed in Table 10, can be used to unambiguously identify one of the three discrete RCC specific states which for sake of nomenclature has been named B herein. More precisely, if genes 1 to 286 of Table 10 are found to be overexpressed and if genes 287 to 454 of Table 10 are found to be underexpressed for a sample of a human or animal individual, the individual will be characterized as having the discrete RCC specific state B. As mentioned before this state is indicative of an intermediate average survival time where about 45 to about 55% such as about 50% of patients can be expected to live after 60 months. Preferably, the presence of this signature will be indicative of a discrete disease-specific state in RCC, which is indicative of an intermediate average survival time where about 40 to about 50% such as about 45% of patients can be expected to live after 90 months.

If, however, it is found that genes 1 to 286 of Table 10 are underexpressed and that genes 287 to 454 of Table 10 are overexpressed, the individual can be diagnosed to display one of the remaining two discrete RCC-specific states, namely A or C.

In order to determine whether such an individual displays states A or C, the expression pattern of the genes listed in Table 11 can be examined. If genes 1 to 19 of Table 11 are overexpressed and if genes 20 to 195 of Table 11 are underexpressed, the individual will display state C which is indicative of a low average survival time where e.g. about 30% to about 45% such as about 40% of patients can be expected to live after 60 months. Preferably, the presence of this signature will be indicative of a discrete disease-specific state in RCC, which is indicative of an intermediate average survival time where about 5 to about 30% such as about 10% to 20% of patients can be expected to live after 90 months.

If, however, genes 1 to 19 of Table 11 are underexpressed and if genes 20 to 195 of Table 11 are overexpressed, the individual will display state A which is indicative of a high average survival time where about 70 to about 90% such as about 80% of patients can be expected to live after 60 months. Preferably, the presence of this signature will be indicative of a discrete disease-specific state in RCC, which is indicative of an intermediate average survival time where about 60 to about 80% such as about 70% of patients can be expected to live after 90 months.

Expression levels may be determined using the Affymetrix gene chips HG-U133A, HG-U133B, HG-U133_Plus_{—}2, etc. The decision as to whether a certain gene in a specific sample is over- or underexpressed will be taken in comparison to a control. This control will be either implemented in the software, or an overall median or other arithmetic mean across measurements is built. By implying a multitude of samples it is also conceivable to calculate a median and/or mean for each gene respectively. In relation to these results, a respective gene expression value is monitored as up or down-regulated.

It is to be understood that the RCC signatures as they are defined by the expression patterns of the genes of Tables 10 and 11 reflect the outcome of a statistical analysis across multiple samples.

For the methods of diagnosis, prognosis, stratification, determining responsiveness etc. as described herein, one will usually test samples obtained from an individual. On the individual level, the expression level of a single gene of Table 10 and/or 11 may not necessarily be sufficient to unambiguously allocate a discrete RCC specific state as the individual may not e.g. overexpress this single gene. Therefore, one will usually analyze the expression pattern of more than one gene of Tables 10 and 11.

Typically one will analyze the expression pattern of at least about 6, at least about 7, at least about 8, at least about 9, at least about 10, at least about 11, at least about 12, at least about 13, at least about 14, at least about 15, at least about 16, at least about 17, at least about 18, at least about 19 or at least about 20 genes of Table 10 to decide on whether the discrete RCC specific state being labeled herein as B is present or not. The analysis of the expression pattern of at least 6 genes of Table 10 will allow deciding whether state B or state A or C is present with a reliability of about 60% or more. This reliability will increase if more genes are analyzed. Thus, the analysis of the expression pattern of at least 10 genes of Table 10 will allow deciding whether state B or state A or C is present with a reliability of about 80% or more. The analysis of the expression pattern of at least 15 genes of Table 10 will allow deciding whether state B or state A or C is present with a reliability of about 90% or more and the analysis of the expression pattern of at least 20 genes of Table 10 will allow deciding whether state B or state A or C is present with a reliability of about 99% or more. The set of about 454 genes of Table 10 thus serves as a reservoir for the unambiguous characterization of state B. By analyzing the expression behavior of e.g. approximately 10 genes of this reservoir, one will be able to decide with a reliability of at least 80% (i) on whether a patient suffers from RCC and (ii) whether the patient suffers from cancer of state B or any of the two other states A or C which will allow to make a prognosis as to the average survival time. In order to differentiate between states A and C, one then has to analyze the expression pattern of genes of Table 11.

Similarly, one will analyze the expression pattern of at least about 6, at least about 7, at least about 8, at least about 9, at least about 10, at least about 11, at least about 12, at least about 13, at least about 14, at least about 15, at least about 16, at least about 17, at least about 18, at least about 19 or at least about 20 genes of Table 11 to decide on whether the discrete RCC specific state being labeled herein as A or C is present. The analysis of the expression pattern of at least 5 genes of Table 11 will allow deciding whether state A or C is present with a reliability of about 60% or more. This reliability will increase if more genes are analyzed. Thus, the analysis of the expression pattern of at least 10 genes of Table 11 will allow deciding whether state A or C is present with a reliability of about 80% or more. The analysis of the expression pattern of at least 15 genes of Table 11 will allow deciding whether state A or C is present with a reliability of about 90% or more and the analysis of the expression pattern of at least 20 genes of Table 11 will allow deciding whether state A or C is present with a reliability of about 99% or more.

The present invention thus relates to a signature, which can be derived from the expression pattern of at least about 6, at least about 7, at least about 8, at least about 9, at least about 10, at least about 11, at least about 12, at least about 13, at least about 14, at least about 15, at least about 16, at least about 17, at least about 18, at least about 19 or at least about 20 genes of Table 10. This signature will allow to unambiguously decide whether one of three discrete RCC specific states, namely state B is present. This signature is defined by an over expression of genes 1 to 286 and an underexpression of genes 287 to 454 of Table 10. The signature which is defined by an underexpression of genes 1 to 286 and an overexpression of genes 287 to 454 of Table 10 is indicative of the two other states of RCC, namely A or C.

The invention also relates to a signature, which can be derived from the expression pattern of at least about 6, at least about 7, at least about 8, at least about 9, at least about 10, at least about 11, at least about 12, at least about 13, at least about 14, at least about 15, at least about 16, at least about 17, at least about 18, at least about 19 or at least about 20 genes of Table 11. This signature will allow to unambiguously decide which of the two remaining discrete RCC specific states, namely states A or C is present. The signature which is defined by an over expression of genes 1 to 19 and an underexpression of genes 20 to 195 of Table 11 is indicative of state C. The signature which is defined by an underexpression of genes 1 to 19 and an overexpression of genes 20 to 195 of Table 11 is indicative of state A.

The present invention also relates to the above signatures for use as a diagnostic and/or prognostic marker in the context of RCC. By determining whether the signatures are present, one can take a decision as to whether a patient suffers from RCC as such and/or will likely develop RCC as such in the future. Further, one can distinguish between the aggressiveness of RCC development and adjust therapy accordingly. Further, the present invention relates to the above signatures for use in stratifying test populations for clinical trials for treatment of RCC.

Further, the present invention relates to the above signatures for use as a read out of a target for development, identification and/or screening of at least one pharmaceutically active compound in the context of RCC as described above.

The present invention also relates to the above signatures for use in stratifying human or animal individuals which are suspected to suffer from ongoing or imminent RCC development. Stratification allows to group these individuals by their discrete RCC specific states. Potential pharmaceutically active compounds which are assumed to be effective in RCC treatment can thus be analyzed in such pre-selected patient groups.

The present invention in one embodiment also relates to a method of diagnosing, prognosing, stratifying and/or screening renal cell carcinoma in at least one human or animal patient, which is suspected of being afflicted by said disease, comprising at least the steps of:

a. Providing a sample of a human or animal individual being suspected to suffer from renal cell carcinoma;

b. Testing said sample for a signature indicative of a discrete renal cell carcinoma specific state by determining expression of at least 6, preferably at least 10 genes of Table 10;

c. Allocating a discrete renal cell carcinoma specific state to said sample based on the signature determined in step b.).

Further, the present invention in one embodiment relates to a method of determining the responsiveness of at least one human or animal individual, which is suspected of being afflicted by renal cell carcinoma, towards a pharmaceutically active agent comprising at least the steps of:

a. Providing a sample of a human or animal individual being suspected to suffer from renal cell carcinoma before the pharmaceutically active agent is administered;

b. Testing said sample for a signature indicative of a discrete renal cell carcinoma specific state by determining expression of at least 6, preferably at least 10 genes of Table 10;

c. Allocating a discrete renal cell carcinoma-specific state to said sample based on the signature determined in step b.);

d. Determining the effect of the pharmaceutically active agent on the disease symptoms and/or discrete renal cell carcinoma-specific states in said individual;

e. Identifying a correlation between the effects on disease symptoms and/or discrete renal cell carcinoma-specific states and the initial discrete renal cell carcinoma-specific state of the sample as determined in step c).

In yet another embodiment, the invention relates to a method of predicting the responsiveness of at least one patient which is suspected of being afflicted by renal cell carcinoma, towards a pharmaceutically active agent comprising at least the steps of:

a. Determining whether a correlation between effects on disease symptoms and/or discrete renal cell carcinoma-specific states and the initial discrete renal cell carcinoma-specific state as a consequence of administration of a pharmaceutically active agent exists by using the above method;

b. Testing a sample of a human or animal individual patient which is suspected of being afflicted by renal cell carcinoma for a signature indicative of a discrete renal cell carcinoma specific state by determining expression of at least 6, preferably of at least 10 genes of Table 10;

c. Allocating a discrete disease-specific state to said sample based on the signature determined in step c.);

d. Comparing the discrete renal cell carcinoma-specific state of the sample in step c. vs. the discrete renal cell carcinoma-specific state for which a correlation has been determined in step a.);

e. Predicting the effect of a pharmaceutically active compound on the disease symptoms in said patient.

One embodiment of the invention relates to a method of determining the effects of a potential pharmaceutically active agent for treatment of renal cell carcinoma, comprising at least the steps of:

a. Providing a sample of a human or animal individual being suspected to suffer from renal cell carcinoma before a pharmaceutically active agent is applied;

b. Testing said sample for a signature indicative of a discrete renal cell carcinoma specific state by determining expression of at least 6, preferably of at least 10 genes of Table 10;

c. Allocating a discrete renal cell carcinoma specific state to said sample based on the signature determined in step b.);

d. Providing a sample of a human or animal individual being suspected to suffer from renal cell carcinoma after a pharmaceutically active agent is applied;

e. Testing said sample for a signature indicative of a discrete renal cell carcinoma specific state by determining expression of at least 6, preferably of at least 10 genes of Table 10;

f. Allocating a discrete renal cell carcinoma specific state to said sample based on the signature determined in step e.);

g. Comparing the discrete renal cell carcinoma specific states identified in steps c.) and f.).

In these methods, one signature is characterized by the expression pattern of at least 6, 7, 8, or 9, preferably of at least 10, 11, 12, 13, 14, 15, 16, 17, 18, 19 or 20 genes of Table 10 with genes 1 to 286 of Table 10 being overexpressed and genes 287 to 454 of Table 10 being underexpressed. This signature is indicative of discrete RCC specific state B. The signature is thus indicative of an RCC type with an intermediate average survival time where about 45 to about 55% such as about 50% of patients can be expected to live after 60 months. Preferably, the presence of this signature will be indicative of a discrete disease-specific state in RCC, which is indicative of an intermediate average survival time where about 40 to about 50% such as about 45% of patients can be expected to live after 90 months.

In these methods, one signature is characterized by the expression pattern of at least 6, 7, 8, or 9, preferably of at least 10, 11, 12, 13, 14, 15, 16, 17, 18, 19 or 20 genes of Table 10 with genes 1 to 286 of Table 10 being underexpressed and genes 287 to 454 of Table 10 being overexpressed. This signature is indicative of the discrete RCC specific states A or C. For an unambiguous differentiation, one may rely on signatures based on the expression profile of genes of Table 11.

Such signatures may be characterized by the expression pattern of at least 6, 7, 8 or 9, preferably of at least 10, 11, 12, 13, 14, 15, 16, 17, 18, 19 or 20 genes of Table 11 with genes 1 to 19 of Table 11 being overexpressed and genes 20 to 195 of Table 11 being underexpressed. This signature is indicative of discrete specific RCC state C. It thus indicates an RCC type with a low average survival time where e.g. about 30% to about 45% such as about 40% of patients can be expected to live after 60 months. Preferably, the presence of this signature will be indicative of a discrete disease-specific state in RCC, which is indicative of an intermediate average survival time where about 5 to about 30% such as about 10% to 20% of patients can be expected to live after 90 months.

Another signature may be characterized by the expression pattern of at least 6, 7, 8, or 9, preferably of at least 10, 11, 12, 13, 14, 15, 16, 17, 18, 19 or 20 genes of Table 11 with genes 1 to 19 of Table 11 being underexpressed and genes 20 to 195 of Table 11 being overexpressed. This signature is indicative of discrete specific RCC state A. It thus indicates an RCC type with a high average survival time where about 70 to about 90% such as about 80% of patients can be expected to live after 60 months. Preferably, the presence of this signature will be indicative of a discrete disease-specific state in RCC, which is indicative of an intermediate average survival time where about 60 to about 80% such as about 70% of patients can be expected to live after 90 months.

As mentioned above the set of genes in Tables 10 and 11 were identified by computer-implemented, algorithm-based approaches after it had been shown that three discrete disease specific states exist in the case of RCC. With this knowledge at hand, it was speculated that computer-implemented, algorithm-based approaches can be used to identify such patterns in existing expression data. Such an approach is described in the Example section under “3. Identification of RCC specific gene sets”.

The invention in some embodiments thus relates to:

1. Discrete disease-specific state for use as a diagnostic and/or prognostic marker in classifying a sample from at least one patient, which is suspected of being afflicted by a disease, optionally by a hyper-proliferative disease.

2. Discrete disease-specific state for use as a diagnostic and/or prognostic marker in classifying a least one cell line of a disease, optionally of a hyper-proliferative disease.

3. Discrete disease-specific state for use as a target for development, identification and/or screening of at least one pharmaceutically active compound.

4. Discrete disease-specific state according to any of 1 to 3, which can be described by way of a signature of at least one descriptor.

5. Discrete disease-specific state according to 4, wherein said state can be described by way of a signature which comprises at least two descriptors which have been identified by comparing at least two regulatory networks in at least two patient derived-samples or cell lines.

6. Signature for use as a diagnostic and/or prognostic marker in classifying at least sample from at least one patient which is suspected to be afflicted by a disease, optionally by a hyper-proliferative disease wherein the signature comprises a qualitative and/or quantitative pattern of at least one descriptor and wherein the signature is indicative of a discrete disease-specific state.

7. Signature for use as a diagnostic and/or prognostic marker in classifying at least one cell line of at least one disease, optionally of a hyper-proliferative disease, wherein the signature comprises a qualitative and/or quantitative pattern of at least one descriptor and wherein the signature is indicative of a discrete disease-specific state.

8. Signature for use as a read out of a target for development, identification and/or screening of at least one pharmaceutically active compound, wherein the signature comprises a qualitative and/or quantitative pattern of at least one descriptor and wherein the signature is indicative of a discrete disease-specific state.

9. Signature according to any of 6 to 8, which can be identified by analyzing multiple descriptors from at least two different regulatory networks in at least two patient-derived samples or in at least two different cell lines.

10. Signature according to 9, which can be identified by analyzing approximately 200 to 400 descriptors from approximately 76 regulatory pathways in approximately 100 patient derived samples or approximately 20 cell lines.

11. Signature according to 9, which is identified by analyzing approximately 200 to 400 descriptors from approximately 165 regulatory pathways in approximately 100 patient derived samples or approximately 20 cell lines.

12. Signature according to any of 6 to 11, wherein the localization, the processing, the modification, the kinetics and/or the expression pattern of descriptors serves as a signature.

13. Signature according to any of 6 to 12, wherein genes or gene-associated molecules are used as descriptors and wherein the expression pattern thereof serves as a signature.

14. Signature according to 13, wherein expression is tested on the RNA or protein level.

15. Signature according to any of 6 to 14 for use as diagnostic and/or prognostic marker in the classification of at least one disease, optionally of at least one hyper-proliferative disease, preferably of renal cell carcinoma, or for use as read out of a target for development, identification and/or screening of at least one pharmaceutically active compound, wherein the signature is characterized by:
an overexpression of at least one gene of table 1, and/or
an underexpression of at least one gene of table 2.

16. Signature according to 15, wherein the signature is characterized by:
a. an overexpression of at least one gene of table 1, and/or
b. an underexpression of at least one gene of table 2, and wherein determination of the over- and/or underexpression of at least one gene of table 1 and table 2 respectively allows assigning a discrete disease-specific state with a likelihood of more than 50%.

17. Signature according to 16, wherein the signature is characterized by:
a. an overexpression of at least one gene of table 1, and/or
b. an underexpression of at least one gene of table 2, and wherein determination of the over- and/or underexpression of at least four genes of table 1 and table 2 respectively allows assigning a discrete disease-specific state with a likelihood of ≧95%.

18. Signature according to any of 15 to 17, wherein the signature is indicative of a discrete disease-specific state at least in RCC, which is indicative of an intermediate average survival time where about 45 to about 55% of patients can be expected to live after 60 months.

19. Signature according to any of 7 to 14 for use as diagnostic and/or prognostic marker in the classification of at least one disease, optionally of at least one hyper-proliferative disease, preferably of renal cell carcinoma, or for use as a read out of a target for development, identification and/or screening of at least one pharmaceutically active compound, wherein the signature is characterized by:
a. an overexpression of at least one gene of table 3, and/or
b. an underexpression of at least one gene of table 4.

20. Signature according to 19, wherein the signature is characterized by:
a. an overexpression of at least one gene of table 3, and/or
b. an underexpression of at least one gene of table 4, and wherein determination of the over- and/or underexpression of at least one gene of table 3 and table 4 respectively allows assigning a discrete disease-specific state with a likelihood of ≧50%.

21. Signature according to 20, wherein the signature is characterized by:
a. an overexpression of at least one gene of table 3, and/or
b. an underexpression of at least one gene of table 4, and wherein determination of the over- and/or underexpression of at least six genes of table 3 and table 4 respectively allows assigning a discrete disease-specific state with a likelihood of ≧95%.

22. Signature according to any of 19 to 21, wherein the signature is indicative of a discrete disease-specific state at least in RCC, which is indicative of a low average survival time where about 35 to about 45% of patients can be expected to live after 60 months.

23. Signature according to any of 7 to 14 for use as diagnostic and/or prognostic marker in the classification of at least one disease, optionally of at least one hyper-proliferative disease, preferably of renal cell carcinoma, or for use as a read out of a target for development, identification and/or screening of at least one pharmaceutically active compound, wherein the signature is characterized by:
a. an underexpression of at least one gene of table 3, and/or
b. an overexpression of at least one gene of table 4.

24. Signature according to 23, wherein the signature is characterized by:
a. an underexpression of at least one gene of table 3, and/or
b. an overexpression of at least one gene of table 4, and wherein determination of the under- and/or overexpression of at least one gene of table 3 and table 4 respectively allows assigning a discrete disease-specific state with a likelihood of ≧50%.

25. Signature according to 24, wherein the signature is characterized by:
a. an underexpression of at least one gene of table 3, and/or
b. an overexpression of at least one gene of table 4, and wherein determination of the under- and/or overexpression of at least six genes of table 3 and table 4 respectively allows assigning a discrete disease-specific state with a likelihood of ≧95%.

26. Signature according to any of 23 to 25, wherein the signature is indicative of a discrete disease-specific state at least in RCC, which is indicative of a high average survival time where about 70 to about 90% can be expected to live after 60 months.

27. Signature according to any of 7 to 26, wherein the signature is indicative of a discrete disease-specific state that is indicative of a functional clinical parameter such as survival time.

28. Method of identifying a signature and optionally at least one discrete disease-specific state being implicated in at least one disease, optionally in at least one hyper-proliferative disease comprising at least the steps of:
a. Testing for quality and/or quantity of descriptors of genes or gene associated molecules in disease-specific samples derived from human or animal individuals suffering from said disease or in cell lines of said disease;
b. Clustering the results obtained in step a.) comprising at least the steps of:
   i. Sorting the results for each descriptor by its quality and/or quantity,
   ii. Sorting the disease-specific samples or cell lines for comparable quality and/or quantity of descriptors across all descriptors;
   iii. Identifying different patterns for common sets of descriptors;
   iv. Allocating to each pattern identified in step b.)iii.) a signature;
   v. Optionally allocating to each signature identified in step b.),iv.) a discrete disease-specific state.

29. Method according to 28, comprising at least the steps of:
a. Testing for quality and/or quantity of descriptors of genes or gene associated molecules in disease-specific samples derived from human or animal individuals suffering from said disease or in cell lines of said disease;
b. Clustering the results obtained in step a.) comprising at least the steps of:
   i. Sorting the results for each descriptor by its quality and/or quantity,
   ii. Sorting the disease-specific samples or cell lines for comparable quality and/or quantity of descriptors across all descriptors;
   iii. Identifying different groups of descriptors which are differentially regulated across said disease-specific samples or cell lines;
c. Combining the descriptors which are identified in step b.)iii.) wherein the quality and/or quantity of said descriptors disease-specific samples or cell lines are already known from step a.);
d. Clustering the results obtained in step c.) comprising at least the steps of:
   i. Sorting the results for each descriptor of step c.) by its quality and/or quantity,
   ii. Sorting the disease-specific samples or cell lines for comparable quality and/or quantity of descriptors across all descriptors;
   iii. Identifying different patterns for the set of descriptors obtained in step c.);
   iv. Allocating to each pattern identified in step d.)iii.) a signature;
   v. Optionally allocating to each signature identified in step d.),iv.) a discrete disease-specific state.

30. Method according to 28 or 29, comprising at least the steps of:
a. Testing for quality and/or quantity of descriptors of genes or gene associated molecules which are associated with at least two regulatory networks in disease-specific samples derived from human or animal individuals suffering from said disease or in cell lines of said disease;
b. Clustering the results obtained in step a.) comprising at least the steps of:
   i. Sorting the results for each descriptor within at least one regulatory network by its quality and/or quantity,
   ii. Sorting the disease-specific samples or cell lines for comparable quality and/or quantity of descriptors across all descriptors within one regulatory network;
   iii. Identifying different groups of descriptors which are differentially regulated across said disease-specific samples or cell lines within the at least one regulatory network;
c. Combining the descriptors which are identified in step b.)iii.) wherein the quality and/or quantity of said descriptors of disease-specific samples or cell lines are already known from step a.);
d. Clustering the results obtained in step c.) comprising at least the steps of:
   i. Sorting the results for each descriptor of step c.) by its quality and/or quantity,
   ii. Sorting the disease-specific samples or cell lines for comparable quality and/or quantity of descriptors across all descriptors;
   iii. Identifying different patterns for the set of descriptors obtained in step c.);
   iv. Allocating to each pattern identified in step d.)iii.) a signature;
   v. Optionally allocating to each signature identified in step d.),iv.) a discrete disease-specific state.

31. Method according to 30, wherein approximately 200 to 400 descriptors from approximately 76 regulatory pathways in approximately 100 patient derived samples or approximately 20 cell lines are analyzed.

32. Method according to 31, wherein approximately 200 to 400 descriptors from approximately 165 regulatory pathways in approximately 100 patient derived samples or approximately 20 cell lines are analyzed.

33. Method according to any of 28 to 32, wherein the localization, the processing, the modification, the kinetics and/or the expression pattern of descriptors serves as a signature.

34. Method according to any of 28 to 33, wherein genes or gene-associated molecules are used as descriptors and wherein the expression pattern thereof serves as a signature.

35. Method according to 34, wherein expression is tested on the RNA or protein level.

36. Method according to any of 30 to 35, wherein the regulatory networks are those identifiable by the Panther Software.

37. Method according to any of 28 to 36, wherein the clustering process in steps b. or d. of claims 28 to 30 is a two-way hierarchical clustering with the TIGR MeV software.

38. Method according to any of 28 to 37, wherein the identification of groups and signatures process in steps b. or d. of claims 28 to 30 is done with the SAM software.

39. Method according to any of 28 to 38, wherein the disease-specific samples are renal cell carcinoma cell lines.

40. Method according to any of 28 to 38, wherein the cell lines are primary or permanent renal cell carcinoma cell lines.

41. Methods according to any of 28 to 40, wherein the discrete disease specific states and the signatures describing them can be linked to functional clinical parameters such as survival time.

42. A set of descriptors obtainable by a method of any of 28 to 41.

43. A signature obtainable by a method of any of 28 to 41.

44. A discrete disease-specific state obtainable by a method of any of 28 to 41.

45. Use of a set of descriptors of 42, a signature of 43 and/or a discrete disease-specific sample of 44 as a diagnostic or prognostic marker for at least one disease, optionally at least one hyper-proliferative disease or as a read out of a target or as a target for the development and/or application of at least one pharmaceutically active compound.

46. A method of diagnosing, stratifying and/or screening a disease, optionally a hyper-proliferative disease in at least one patient, which is suspected of being afflicted by a disease, optionally by a hyper-proliferative disease or in at least one cell line of a disease, optionally of a hyper-proliferative disease comprising at least the steps of:
a. Providing a sample of a human or animal individual being suspected to suffer from said disease, optionally of said hyper-proliferative disease or at least one cell line of said disease, optionally of said hyper-proliferative disease;
b. Testing said sample for a signature, optionally a signature of 43;
c. Allocating a discrete disease-specific state to said sample or cell line based on the signature determined in step b.).

47. A method of determining the responsiveness of at least one human or animal individual which is suspected of being afflicted by a disease, optionally by a hyper-proliferative disease towards a pharmaceutically active agent comprising at least the steps of:
a. Providing a sample of at least one human or animal individual which is suspected of being afflicted by a disease before the pharmaceutically active agent is administered;
b. Testing said sample for a signature, optionally a signature of 43;
c. Allocating a discrete disease-specific state to said sample based on the signature determined;
d. Determining the effect of a pharmaceutically active compound on the disease symptoms and/or discrete disease-specific state in said individual;
e. Identifying a correlation between the effects on disease symptoms and/or discrete disease-specific state and the discrete disease-specific state of the sample.

48. A method of predicting the responsiveness of at least one patient which is suspected of being afflicted by a disease, optionally by a hyper-proliferative disease towards a pharmaceutically active agent comprising at least the steps of:
a. Determining whether a correlation between effects on disease symptoms as a consequence of administration of a pharmaceutically active agent and a discrete disease-specific state exists by using the method of 46;
b. Testing a sample of a human or animal individual which is suspected of being afflicted by a disease, optionally by a hyper-proliferative disease for a signature, optionally a signature of 43;
c. Allocating a discrete disease-specific state to said sample based on the signature determined;
d. Comparing the discrete disease-specific state of the sample in step c. vs. the discrete disease-specific state for which a correlation has been determined in step a.);
e. Predicting the effect of a pharmaceutically active compound on the disease symptoms in said patient.

49. A method of determining the effects of a potential pharmaceutically active compound, comprising at least the steps of:
a. Providing a sample of at least one human or animal individual which is suspected of being afflicted by a disease, optionally by a hyper-proliferative disease or a cell line of a disease, optionally of a hyper-proliferative disease before a pharmaceutically active agent is applied;
b. Testing said sample or cell line for a signature, optionally a signature of 43;
c. Allocating a discrete disease-specific state to said sample or cell line based on the signature determined;
d. Providing a sample of at least one human or animal individual which is suspected of being afflicted by a disease, optionally by a hyper-proliferative disease or a cell line of a disease, optionally of a hyper-proliferative disease after a pharmaceutically active agent is applied;
e. Testing said sample or cell line for a signature, optionally a signature of 43;
f. Allocating a discrete disease-specific state to said sample or cell line based on the signature determined;
g. Comparing the discrete disease-specific state identified in steps c.) and f.).

50. A method of any of 46 to 49, wherein said discrete disease-specific states are determined for samples of a patient being suspected of suffering from renal cell carcinoma or for renal cell carcinoma cell lines.

51. A method of any of 46 to 50, wherein a discrete disease specific state of a disease, optionally of a hyper-proliferative disease, preferably of renal cell carcinoma is allocated by a signature, wherein the signature is characterized by:
a. an overexpression of at least one gene of table 1, and/or
b. an underexpression of at least one gene of table 2.

52. Method of 51, wherein the signature is characterized by:
a. an overexpression of at least one gene of table 1, and/or
b. an underexpression of at least one gene of table 2, and wherein determination of the over- and/or underexpression of at least one gene of table 1 and table 2 respectively allows assigning a discrete disease-specific state with a likelihood of ≧50%.

53. Method of 52, wherein the signature is characterized by:
a. an overexpression of at least one gene of table 1, and/or
b. an underexpression of at least one gene table 2, and wherein determination of the over- and/or underexpression of at least four genes of table 1 and table 2 respectively allows assigning a discrete disease-specific state with a likelihood of ≧90%.

54. Method according to any of 51 to 53, wherein the signature is indicative of a discrete disease-specific state at least in RCC, which is indicative of an intermediate average survival time where about 45 to about 55% of patients can be expected to live after 60 months.

55. A method of any of 46 to 50, wherein a discrete disease specific state of renal cell carcinoma is allocated by a signature, wherein the signature is characterized by:
a. an overexpression of at least one gene of table 3, and/or
b. an underexpression of at least one gene of table 4.

56. A method of 55, wherein the signature is characterized by:
a. an overexpression of at least one gene of table 3, and/or
b. an underexpression of at least one gene of table 4, and wherein determination of the over- and/or underexpression of at least one gene of table 3 and table 4 respectively allows assigning a discrete disease-specific state with a likelihood of ≧50%.

57. A method of 56, wherein the signature is characterized by:
a. an overexpression of at least one gene of table 3, and/or
b. an underexpression of at least one gene of table 4, and wherein determination of the over- and/or underexpression of at least six genes of table 3 and table 4 respectively allows assigning a discrete disease-specific state with a likelihood of ≧95%.

58. Method according to any of 55 to 57, wherein the signature is indicative of a discrete disease-specific state at least in RCC, which is indicative of a low average survival time where about 35 to 45% of patients can be expected to live after 60 months.

59. A method of any of 46 to 50, wherein a discrete disease specific state of renal cell carcinoma is allocated by a signature, wherein the signature is characterized by:
a. an underexpression of at least one gene of table 3, and/or
b. an overexpression of at least one gene of table 4.

60. A method of 59, wherein the signature is characterized by:
a. an underexpression of at least one gene of table 3, and/or
b. an overexpression of at least one gene of table 4, and wherein determination of the under- and/or overexpression of at least one gene of table 3 and table 4 respectively allows assigning a discrete disease-specific state with a likelihood of ≧50%.

61. A method of 60, wherein the signature is characterized by:
a. an underexpression of at least one gene of table 3, and/or
b. an overexpression of at least one gene of table 4, and wherein determination of the under- and/or overexpression of at least six genes of table 3 and table 4 respectively allows assigning a discrete disease-specific state with a likelihood of ≧95%.

62. Method according to any of 60 to 62, wherein the signature is indicative of a discrete disease-specific state at least in RCC, which is indicative of a high average survival time where about 70 to about 90% of patients can be expected to live after 60 months.

63. A method according to any of 46 to 62, wherein the signature is indicative of a discrete disease-specific state that is indicative of a functional clinical parameter such as survival time.

The invention is now described with respect to specific experiments. These experiment shall, however, not be construed as being limiting.

### Experiments

### 1. Materials and Methods

### Tissue Specimens, Cell Lines, Nucleic Acid Extraction

Frozen primary renal cell carcinoma (RCC) and tissue from RCC metastases were obtained from the tissue biobank of the University Hospital Zurich. This study was approved by the local commission of ethics (ref number StV 38-2005). All tumors were reviewed by a pathologist specialized in uropathology, graded according to a 3-tiered grading system (14) and histologically classified according to the World Health Organisation classification (15). All tumor tissues were selected according to the histologically verified presence of at least 80% tumor cells. DNA was extracted from 56 ccRCC, 13 pRCC and 69 matched normal renal tissues using the Blood and Tissue Kit (Qiagen). RNA was extracted from 74 ccRCC, 22 pRCC, 2 chromophobe RCC, 15 metastases of ccRCC using the RNeasy minikit (Qiagen). DNA and RNA from 46 ccRCC and 10 pRCC were used for both SNP array and microarray experiments. Expression analysis was additionally performed with RNA from 24 RCC cell lines, 6 cell lines from RCC metastasis and 4 prostate cancer cell lines as controls. All tumours and cell lines used in SNP- and expression array experiments are listed in table 6.

### SNP Array Analysis and Classification

SNP array analysis was performed with Genome Wide Human SNP 6.0 arrays according to manufacturer's instructions (Affymetrix). Arrays were scanned using the GeneChip Scanner 3000 7G.

Raw probe data CEL files were processed with the R statistical software framework (http://www.cran.org), using the array analysis packages from the aroma.affymetrix project (16) (http://groups.google.com/group/aroma-affymetrix/). Total copy number estimates were generated using the CRMAv2 method (17) including allelic cross talk calibration, normalization for probe sequence effects and normalization for PCR fragment-length effects. Copy number segmentation was performed using the Circular Binary Segmentation method (18), implemented in the DNA copy package available through the Bioconductor project (http://www.bioconductor.org). Normalized data plots including segmentation results, oncogene map positions and known copy number variations as reported in the Database of Genomic Variants (DGV, http://projects.tcag.ca/variation/; (19)) were generated with software packages developed for the Progenetix project(2) (http://www.progenetix.net). Map positions were referenced with respect to the UCSC genome assembly hg18, based on the March 2006 human reference sequence (NCBI Build 36.1). Data from arrays with prominent probe level noise after normalization were excluded before proceeding with the evaluation of copy number imbalances. Overall, 114 SNP 6.0 arrays (45 tumors, 69 normal tissue samples) were used for final data processing.

Since oncogenomic imbalances frequently cover huge genomic regions with hundreds of possible target genes, a dynamic thresholding approach was used on the copy number segmentation data. For the determination of focussed genomic imbalances containing oncogenetic targets, size-limited regions with high deviation from the copy number baseline were evaluated for their gene content. Primary candidate genes were selected from copy number imbalanced regions if no corresponding full-overlap CNV had been reported in DGV. For the generation of overall genomic imbalance profiles, probabilistic thresholds of 0.13/−0.13 were used for genomic gains and losses, respectively. Recurrently appearing candidates were listed and considered only once for further analysis. Functional gene classifications were performed with Ingenuity (http://www.ingenuity.com), KEGG (Kyoto Encyclopedia of Genes and Genomes—http://www.genome.jp/kegg/) and PANTHER (Protein Analysis Through Evolutionary Relationships) Classification System (20, 21) (http://www.pantherdb.org). Generation and analysis of gene/protein lists were performed with PANTHER by considering both, PubMed & Celera, datasets.

### Microarrays and Expression Analysis

RNA was hybridized according to the manufacturer's instructions (Affymetrix, Santa Clara, Calif.). Arrays were scanned using the HT Scanner. Affymetrix GeneChip data was normalized using MASS from Bioconductor (22) and log₂-scaled. Hierarchical clustering was done with TIGR MeV(23) using Euclidian distance and average linkage. The identification of tumor type specific biomarkers was performed using SAM (12). The most significant genes were cross-checked in GENEVESTIGATOR (10, 11) to remove probe sets that had absent calls across all samples.

Probesets could be identified for at least half of the genes from the four pathways extracted from PANTHER (195 probe sets for angiogenesis, 271 for inflammation, 196 for integrin, and 263 for Wnt). For each pathway, a two-way hierarchical clustering of probe sets versus the complete set of expression arrays (147 arrays) was applied. We selected up to four clusters that best represented the overall array clustering in each pathway (FIG. 2, table 8). Finally, a joint clustering of all probe sets from these clusters resulted in the groupings described (FIG. 3, table 5).

Microarray and SNP data have been deposited in GEO under GSE19949 (tentative release: 30.06.2010). Reviewer link: http://www.ncbi.nlm.nih.gov/geo/query/acc.cgi?token=zronrguasyacefq&acc=GS E19949

Raw microarray expression data were generated by using the HG-U133A Affymetrix chip for each sample respectively. For further analysis, these raw data were uploaded into the online, high quality and manually curated expression database and meta-analysis system GENEVESTIGATOR (www.genevestigator.com). As mentioned, a two way hierarchical clusterings were than performed. Genexpressions versus the entire set of samples were clustered. The gene list used for this first clustering was provided by the PANTHER classification system (www.pantherdb.org) and encompassed the entirety of genes belonging to one pathway (see FIG. 2 and FIG. 3). The result of such a clustering is, that tumors with same expression profiles, seen over all probesets entered, reside in close vicinity. Dependent on the presence of recurrent differentially regulated genes in different tumor samples, distinct clustered form throughout the entire tumor cohort. In a second step, probesets representing these formed clusters, were picked and combined into another clustering matrix. The same two way hierarchical clustering conditions was thereafter performed against the same sample cohort. Upon this analysis, tumor groups appeared (FIG. 3).

In a further step the question was raised whether the best gene candidates were already picked in FIG. 3 to enable a clear differentiation between distinct groups. To answer this question out of FIG. 3, 40 tumor samples (Affymetrix HG-U133A, raw data) from different groups were arbitrarily picked for best identifier detection. By using GENVESTIGATOR in combination with the statistical program SAM (Significance Analysis of Microarrays) the best identifiers for the respective group, were calculated (FIG. 4). Thus these 40 arbitrarily chosen samples were statistically analyzed with respect to expression of all 22.000 probesets present on the Affymetrix HG-U133A microarray. The data generated in FIG. 3 and FIG. 4 are absolute expression values.

We used this resulting signature (FIG. 4) as a “marker-signature” for the following meta analysis. For this purpose the expression characteristics of these genes across different tumor studies available from all HG_U133A microarrays in the GENEVESTIGATOR database was confirmed. The values shown here are relative values (right picture of FIGS. 4A and 4B). Here for every Affymetrix tumor chip, a corresponding control was present. The signature appeared in the tumor samples but not in the control. Further, the values shown are mean values. Several expression array chips from one experimental procedure representing a distinct tumortype were overlaid.

### TMA Construction and Immunohistochemistry

We used two tissue micro arrays (TMAs) with tumor tissue from 27 and 254 RCC-related nephrectomy specimen respectively. The samples were retrieved from the archives of the Institute for Surgical Pathology; University Hospital Zurich (Zurich, Switzerland) between the years 1993 to 2007. TMAs were constructed as previously described (24). To sufficiently address tumor heterogeneity, we used 3 punches per tumor for the construction of the TMA with 27 tumor samples (25). One biopsy cylinder per tumour was regarded as sufficient for constructing the TMA with 254 tumors. TMA sections (2.5 μm) on glass slides were subjected to immunohistochemical analysis according to the Ventana (Tucson, Ariz., USA) automat protocols. CD34 (Serotec Ltd.—clone QBEND-10, dilution 1:800), MSH6 (BD Biosciences—clone 44, dilution 1:500) and DEK (BD Biosciences—clone 2, dilution 1:400) stainings were performed and analysed under a Leitz Aristoplan microscope (Leica, Wetzlar, Germany). Tumors were considered MSH6 or DEK positive if more than 1% of tumour cells showed unequivocal nuclear expression. MVD was determined as previously described (26). Statistics were performed with Statview 5.0 (SAS, USA) and SPSS 17.0 for Windows (SPSS Inc., Chicago; IL).

### 2. Results

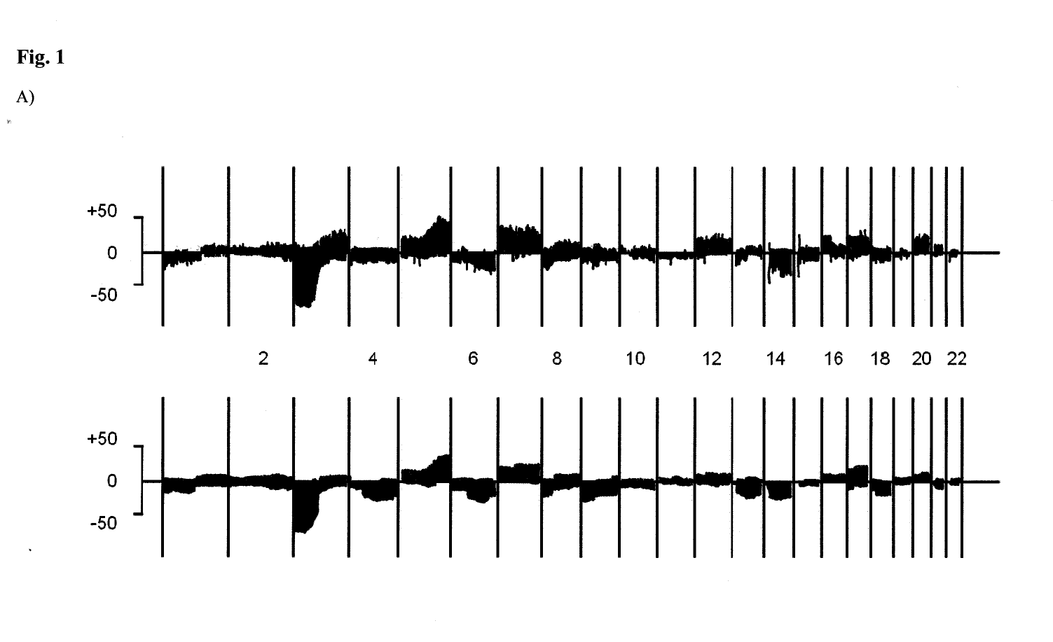

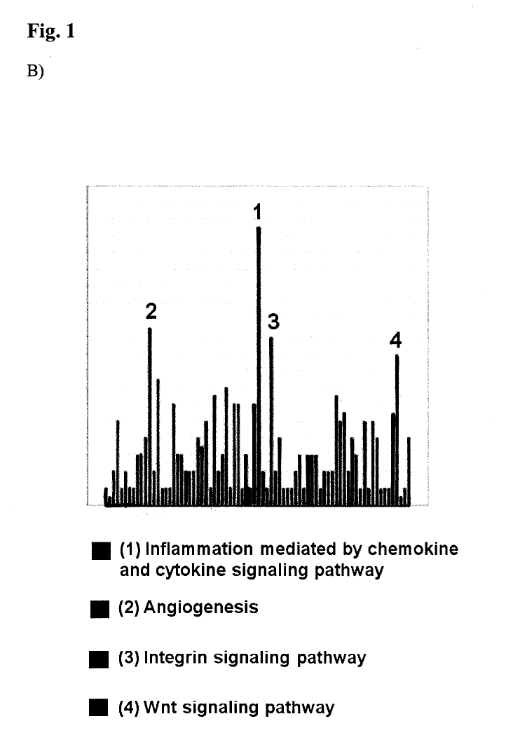

First, the genomic profiles of 45 RCCs and matched normal tissues were analyzed using Affymetrix SNP arrays. For illustration, we extracted an overall summary of genomic imbalances using the progenetix website (http://www.progenetix.net) and compared them to the entire available dataset of 472 RCCs (FIG. 1A). Consistent with previous CGH data (8), our results confirmed the overall composite of CGH profiles in RCC.

We next focused on tumor-specific genomic changes below 5 Mb, which is the resolution limit for chromosomal losses and gains obtained by CGH (9). We identified 126 different regions in our cohort varying between 0.5 kb to 5 Mb and encompassing 61 allelic gains and 65 allelic losses. Irrespective of the type of allelic imbalance and gene function, we assigned the same relevance to each identified region and gene by considering it as “affected”. In total, coding regions of 769 genes were partially or entirely involved and only 5 genes (AUTS2, ETS1, FGD4, PRKCH, FTO) were found recurrently affected in only up to 5 tumors.

In contrast to large chromosomal aberrations commonly detected by CGH in public data, the genomic alterations <5 Mb could not be linked to morphologically defined RCC subtypes. Additional expression analysis of the 769 genes against the GENEVESTIGATOR (10, 11) (http://www.genevestigator.com) human microarray dataset showed no apparent clustering (data not shown). We next ran the entire gene list against classification systems such as Ingenuity, KEGG and PANTHER. The PANTHER software integrated them into superior biological processes. This database mapped 557 of 769 IDs (73%). PANTHER BAR CHART allocated the 557 genes to 76 of a total of 165 available signaling- and metabolic “networks” (FIG. 1B, Table 7). Analyzing the genes for each of these four processes revealed the diversity and plasticity with genes commonly involved in different “pathways”, culminating in superior biological processes. As an example the “Actin related protein ⅔ complex”, initially affiliated to “Inflammation” (PANTHER pathway ID P00031), contains the gene ARPC5L which is also implicated in Integrin signalling (PANTHER pathway ID P00034), Huntington disease (PANTHER pathway ID P00029) or the Cytoskeletal regulation by Rho GTPases (PANTHER pathway ID P00016).

We then generated gene lists of each of the 76 processes as assigned by PANTHER and investigated each of these gene lists on the RNA expression level by hierarchical clustering in 98 primary RCCs (including the samples used for the SNP array experiment), 15 RCC metastases as well as in 34 cell lines, using Affymetrix HG-U133A arrays (Table 6). For example, the four dominating biological processes (FIG. 1B) “Inflammation”, “Angiogenesis”, “Integrin” and “Wnt” consisted of 476, 354, 365 and 497 genes, respectively. Within the clustering of these four dominating processes we observed different, clearly distinguishable major group patterns (FIG. 2A-D, table 5), suggesting several tumor group-specific gene regulatory mechanisms. In contrast, no clear differential gene expression patterns were obtained through hierarchical clustering of the genes of the remaining 72 biological processes (including those for apoptosis, HIF or p53 signaling).

We then selected up to four gene clusters from each of the four matrices with a total of 92 genes that were most representative for the overall clustering of the samples (FIG. 2A-D, red boxes, Table 8) and combined them into a new matrix. Subsequent clustering of this matrix yielded four clearly distinct tumor groups (termed “A”, “B”, “C” and “cell lines”) (FIG. 3, table 5). Although being members of four “pathways” as proposed by PANTHER, the 92 genes represented only a small percentage of genes involved in these biological processes. We therefore preferred to subdivide the tumor groups into “A”, “B” “C” and “cell lines” rather than considering them as pathway-specific. Notably, even though only one (ITGAL) out of the 92 selected cluster-related genes was directly affected by a CNA in only one tumor of our RCC set, they collectively constitute group-specific expression signatures which ultimately appear to have originated from the genomic alterations detected by our SNP array analysis.

In contrast to the cell lines which represent a separate group, RCC metastases and primary RCCs split into group A, B or C irrespective of the tumor subtype, stage or differentiation grade. Although clear cell RCC (ccRCC), papillary RCC (pRCC) and chromophobe RCC have a different morphological phenotype, the combined appearance of the three subtypes across different clusters suggests molecular similarities.

We then profiled gene expression across 40 primary RCC samples that were arbitrarily chosen from the three tumor groups previously identified in FIG. 3. Hierarchical clustering of these samples across all 22,000 probe sets of this array showed that type B was clearly distinct from A and C (FIG. 4C left) and group A appeared as a tight cluster within the C clad. Using SAM (12), at least a 2-fold change in the expression level was seen for more than 2,000 genes, with 1,455 genes higher and 715 genes lower expressed in B compared to A and C, and 221 genes positively and 11 genes negatively regulated in A versus C. These independent findings confirmed the previous grouping of RCCs based on the genes derived from the SNP array results.

The most differentially regulated genes between group B and groups A and C were represented by 48 genes, with 16 being low expressed in B but strongly expressed in A and C (8.7−5.7 fold change) and 32 transcripts being abundant in B but decreased in A and C (14.4−5.2 fold change) (FIG. 4A left, Tables 1 and 2). Twenty-three genes clearly distinguished groups A and C with 4 genes being highly expressed in C but not in A (14.3−2.5 fold change), while 19 were highly expressed in A but not in C (16.0−4.2 fold change) (FIG. 4B left, Tables 3 and 4).

We then compared the expression characteristics of these genes across 80 different tumor studies (comparison sets of “tumor” versus “healthy”) available from all HG_U133A microarrays in the GENEVESTIGATOR database. For those genes differentially expressed between RCC tumors B versus RCC tumors

A and C, four independent kidney cancer experiments and 24 further tumor sets exhibited a very similar bimodal expression signature. Sixteen of these sets had a similar signature as RCC types A and C; eight sets were similar to type B. Similarly, for those genes that were most significantly deregulated between RCC tumors type A versus type C, 16 tumor sets showed similar characteristics in GENEVESTIGATOR. Not only kidney cancer but also thyroid cancer were similar to RCC type A, while 12 other sets, including breast-, bladder- and cervical carcinoma, were highly correlated to type C. For the remaining 39 tumor sets present in the database, none had group A, B or C specific gene signatures. These results validated our approach as they demonstrate high reproducibility of three different general molecular signatures in carcinogenesis, not only in RCC but also in other tumor types, arguing for conforming molecular strategies exploited by a number of different human cancers.

We then randomly selected 27 RCCs from the three respective groups (FIG. 3) and placed them into a small tissue microarray (TMA). A Hematoxylin/Eosin stained TMA section was blindly evaluated by a pathologist. All nine tumors of group A were characterized by high microvessel density (MVD), whereas there were no specific morphologic features in the tumors of groups B and C. To further verify this finding, we immunohistochemically stained the endothelial cell marker CD34 in the 27 RCCs.

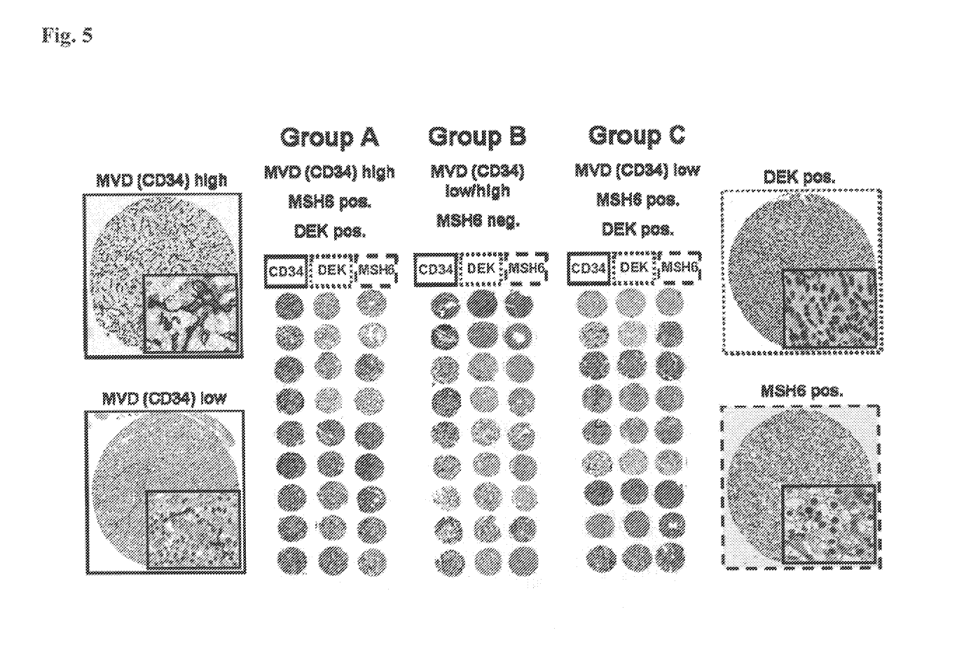

As shown in Table 9 and FIG. 5, the results largely confirmed group-specific angiogenic traits. All nine tumors in group A, but only three in group B and one in group C had more than 100 microvessels, whereas the remaining ones had less than 50 microvessels per arrayed spot (0.036 mm²). Tumors with high and low MVD were classified accordingly. No further specific morphological features were seen in the tumors assigned to group B and C.

We then searched with SAM for genes with a clear present or absent expression profile in the three groups. By examining staining patterns of several protein candidates coded by these genes, we were finally able to assign tumors with high MVD as well as DEK and MSH6 positivity to group A, MSH6 negative tumors to group B, and tumors with low MVD but DEK and MSH6 positivity to group C.

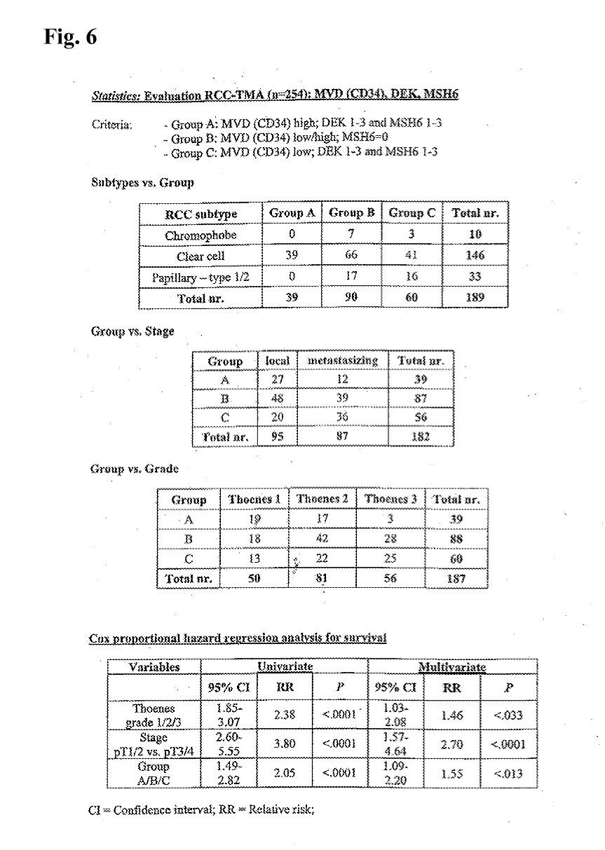

To evaluate the obtained group-specific protein expression patterns in a much higher number of tumors, we screened a TMA with 254 RCCs. By strictly applying the staining combinations obtained form the small test TMA, 189 tumors (75%) were clearly assigned to a specific group. There were organ-confined and metastasizing RCCs of different tumor subtype and nuclear differentiation grade but varying frequencies in these groups (FIG. 6).

To determine the clinical aggressiveness of these groups, we focused our analysis on 176 of 189 RCC samples on the TMA for which survival data were available. Kaplan-Meier analysis showed a highly significant correlation (log rank test: p<0.0001) of group affiliation with overall survival, in which patient outcome was best in group A and worst in group C (FIG. 4D). This result was independent from tumor stage and Thoenes grade in a multivariate analysis (FIG. 6). By performing this survival analysis, we demonstrate that the molecular re-classification of RCC allows the identification of early stage tumors (pT1 and pT2) with high metastasizing potential associated with poor patient prognosis. In addition, the finding of late stage non-metastasizing RCCs in group A also suggests the existence of patients with a relative good prognosis although their tumors were categorized as pT3.

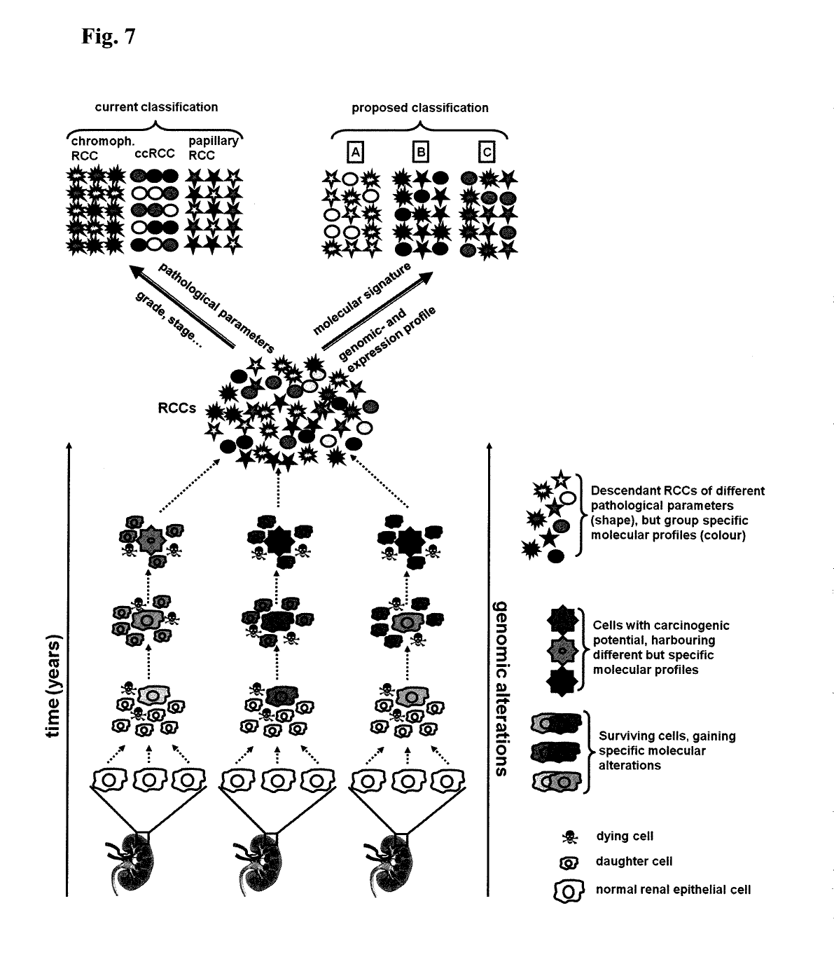

The data presented in this report suggests that the discovered group forming clusters represent gene signatures reflecting three common modalities of cancerogenesis. These gene clusters could be determined only by applying the entire set of the 126 tumor-specific CNAs detected in our RCC cohort. It is therefore remarkable that, although the frequencies of CNAs were largely differing in a single tumor and varied between none and 18 altered genomic regions, each of the group-specific gene expression patterns remained stable. Consequently, each of these RCC must have developed individually balanced mechanisms different to CNAs (i.e. mutations, methylations, transcriptional and translational modifications), which together support the regulation of molecular components to reach one of the three tumor groups (FIG. 7).

Our meta-analysis suggests similar strategies pursued by a number of different human cancer types. It is therefore tempting to speculate that either the entirety of different types of molecular alterations (i.e. mutations, CNAs and methylation) existing in a single tumor or the entirety of a specific type of molecular alteration (i.e. CNAs only) in a tumor type cohort would always lead to group-specific outputs, visualizable by gene expression profiling.

The data indicate that each tumor has programmed its own molecular road map by trial and error to finally reach one of the three different “destinations”. As our meta-analysis demonstrated the existence of these groups or discrete states in different but not in all human cancer types, additional yet unknown groups may exist.

### 3. Identification of RCC Specific Gene Sets

### Expression Data Generation

The data used for the computer-implemented, algorithm-based analysis was created using micro array chips such as those made by Affimetrix. The mRNA in the sample is amplified using PCR. On the micro array chip each gene is represented by multiple (usually 10 to 20) sequences of 25 nucleotides taken from the gene. Usually each sequence is found a second time on the chip in a modified version. This modified version is called mismatch (the correct version is called perfect match). It is used to estimate the unspecific binding of mRNA to the particular sequence. This pair of sequences is called probe pair. All probe pairs for one gene are called probe set. The sequences and their layout across the chip are defined and documented by the vendor of the micro array chip. After each measurement of a sample one has up to 50 values per gene which need to be combined into one expression level for the gene in the sample.

### Normalization

In order to determine the expression level different approaches can be used. One prominent example is the model-based-normalization (27). In model-based-normalization for each probe pair the difference between perfect match (PM) and mismatch (MM) is calculated. One then considers the results for one probe set (in other words: for one gene) but from multiple samples or measurements.

It is assumed that the sensitivity sₚ of each probe pair p is different but specific to this probe pair. On the other hand expression eₛ level for each gene in one sample s should be constant across all probe pairs. Hence one assumes

PM_{s,p}−MM_{s,p}=sₚeₛ+n_{p,s} (1)

where n_{p,s} is some additional noise. sₚ and eₛ are now optimised such that the sum of n_{p,s} is minimal and the sum of sₚ² is equal to the number of probe pairs

An additional way of normalizing data relies on using kernel regression. The rationale for using kernel regression normalization is that probe pair signal and/or expression levels may still exhibit different scaling and offsets across different measurements. For example, the duration and effectiveness of the PCR may modify these signals in this way. Furthermore signals amplification may differ in a non linear way between measurements. In order to compare measurements these modifications have to be compensated. One option for this compensation are kernel regression methods (32), e.g. Lowess.

In order to determine the regression one has to define a set of genes and a reference sample. The reference sample is taken to be the most average sample with the least number of outliers. The gene set may include all genes, but also a subset of genes can be used, e.g. the predefined set of housekeeping genes as supplied by the micro array vendor) or a set of genes determined by the invariant set method (28). However, a systematic amplification of a group of genes due to a cancer status might lead to a similar non-linear relationship between samples. Therefore kernel regression methods shall be used with caution in this context. The software dChip (29) implements most of the aforementioned normalization methods.

### Scaling

The normalization is usually done on a set of samples. Hence these sample become comparable in scale and offset. However, samples measured and normalized at different places will still differ in this respect. Therefore a scaling of the data is required that is robust against
Errors in extreme data points,
Offsets and
Linear scalings.

Furthermore the scaling shall not depend at this point on any data from other samples. One possibility to achieve this is to use the following formula:

e_{scaled}=(f(e)−m)/σ (2)

with
Some function f, which may transform the expression level in order to reduce the influence of extreme value. For example it might be the identity or the logarithm. If a function like the logarithm is used one may need to add a small constant ε before evaluating the logarithm in order to avoid non finite values. Then the size of ε should be of the order of the smallest measured expression levels.
Some average m taken over all expression levels (after transformation by f) within one sample. Examples for this average are the arithmetic average or the median.
Some quantity σ representing the scale of the data. An example here is the standard deviation taken over all expression levels (after transformation by f) within one sample. One may also reduce the range taken into account to the 2 central quartiles.

Another possibility is to scale the expression levels linearly with respect to a set of house keeping genes. These genes shall be selected similarly as for the kernel regression.

### Cluster Search

The states are considered common properties separating one group of tumors from the other both in gene expression levels and medical parameters. These groups of tumors can be established by applying different kinds of methods (33) of unsupervised learning (like neural gases (31) and cluster search, e.g. k-nearest neighbour search (35) to the gene expression profiles of the tumors of a learning set. The selection of distance-measure (a metric) used by the algorithms is also important. One may choose simple euclidean norm but also correlations. The type of scaling used also influences the metric and hence the results of the cluster search.

Therefore it is advisable to use different algorithms, metrics and scalings to get a comprehensive picture from which the states can be derived.

These states may also form a kind of hierarchy, where two sets of states are clearly separated, but themselves split into sub-states.

In the case of RCC cancer 3 states were found, labeled A, B, C. The states A and C are sub states of a more general state which may be designated as AC.

### Gene Search

Once the states are determined one has to find the genes which differentiate one state from the others in a given set of samples. The genes shall be selected in a way that they are as robust as possible against systematic errors of all kinds. This includes a good choice of scaling function as well as good choice of selection criteria. Possible selection criteria are
The Significance Analysis of Microarray-Index (30).
The correlation of the expression levels of all samples in a learning set against a function being 0 for all samples except those being in the state of interest. In latter case this function will be 1.
The correctness of the prediction based on the gene using an optimised single gene model (e.g. see next section).

In order to enhance the quality of gene selection these criteria can be tested on different scalings, e.g. (2) and (4) or different normalizations, e.g. dChip-data and just model-based-normalized data and any combination of these.

The gene search shall only include such genes that fulfill minimum values for all selected criteria, e.g. the absolute of the correlation greater than 0.7 or the correctness greater than 0.85.

### Model

Each sub-model consists of a list of genes g with corresponding thresholds θ_{g} and sides ((1) and (−1)) and two sets of status (set “in” and set “out”). In the first turn each gene is evaluated individually. This list of genes is determined using the gene search mentioned above and genes threshold is determined such that the correctness is optimal. Genes with positive correlation are considered “overexpressed”, the other genes are considered “underexpressed”.

If side is “overexpressed” the following test is done:

if e_{g}>θ_{g}+α increase Nᵢₙ by 1 (3a)

if e_{g}<θ_{g}−α increase Nₒᵤₜ by 1 (3b)

If side is “underexpressed” the following test is done:

if e_{g}<θ_{g}−α increase Nᵢₙ by 1 (3c)

if e_{g}>θ_{g}+α increase Nₒᵤₜ by 1 (3d)

The factor α>=0 defines a range of uncertainty around the genes threshold θ_{g}. A reasonable choice for α is ⅓ if a scale-free scaling (like (2)) was used. Otherwise the scale has to be included into α.

These tests are done for all genes in a sub-model. In the end one has two counts Nᵢₙ and Nₒᵤₜ. Now these two counts are compared:
If Nᵢₙ>βNₐₗₗ and Nᵢₙ>γNₒᵤₜ then the tumor is considered to be in one of the “in” states
Nₒᵤₜ>βNₐₗₗ and Nₒᵤₜ>γNᵢₙ then the tumor is considered to be in one of the “out” states

The factor β defines a minimum fraction of genes which must have taken a decision in (3). The factor γ defines by how much the state set considered must beat the other set of states. Reasonable choices are β=⅓ and γ=2.

### Reduced Models

For some applications the gene lists created by the gene search (see section Gene Search) are too exhaustive. In this case one may use just the best genes as selected by one or more of the criteria mentioned in section Gene Search. But this might not be the best selection for a given number of genes to be used. Although, all genes are tested individually and give a high number of correct predicted states, they may misclassify the same sample unless the genes are selected carefully from the larger list. The smaller the size of the requested subset the more careful the selection has to be done. Therefore an algorithm for sub-selecting genes is required.

This can be done with any optimisation algorithm, such as genetic algorithms or simple random-walk-optimisation on a set of optimisation criteria. Such criteria may include:
Correctness of prediction on the learning set of samples. The result of the full gene list is assumed to be the correct state for the sample. Thus the reduced model is consistent with the full model.
Correctness of the tendencies of prediction on the learning set of samples. If one remove the ranges of uncertainty totally (α=0, β=0, γ=1) or in part (e.g. β=0, γ=1) from the model defined above, one still gets a state but with less reliability. One can call these states tendency of the prediction and use it here if the unchanged model does not predict the state. Again the prediction and tendencies using the full gene list are assumed to represent the correct state for the sample. Thus the reduced model is consistent with the full model both in prediction and tendency.
Errors in prediction and tendencies of test set samples. Thess test set samples have not been included in the original learning set. Such data might be obtained from the Gene Expression Omnibus (34)

Additional constraints (e.g. at least 25% of overexpressed gene) can be applied to the selection algorithm

TABLE 1

| | | | SEQ ID No. | SEQ ID No. |
|---|---|---|---|---|
| No. | Probeset ID* | Gene Symbol | (mRNA) | (amino acid |
| 1 | 216527_at | — | — | — |
| 2 | 214715_x_at | ZNF160 | 1 | 2 |
| 3 | 222368_at | — | — | — |
| 4 | 214911_s_at | BRD2 | 3 | 4 |
| 5 | 214870_x_at | LOC100288442 | 5 | 6 |
| 6 | 215978_x_at | LOC152719 | 7 | 8 |
| 7 | 221501_x_at | LOC339047 | 9 | 10 |
| 8 | 212177_at | SFRS18 | 11 | 12 |
| 9 | 216563_at | ANKRD12 | 13 | 14 |
| 10 | 213311_s_at | TCF25 | 15 | 16 |
| 11 | 216187_x_at | — | — | — |
| 12 | 208246_x_at | — | — | — |
| 13 | 214235_at | CYP3A5 | 17 | 18 |
| 14 | 220796_x_at | SLC35E1 | 19 | 20 |
| 15 | 206792_x_at | PDE4C | 21 | 22 |
| 16 | 214035_x_at | LOC399491 | 23 | 24 |
| 17 | 215545_at | — | — | — |
| 18 | 212487_at | GPATCH8 | 25 | 26 |
| 19 | 221191_at | STAG3L1 | 27 | 28 |
| 20 | 213813_x_at | — | — | — |
| 21 | 220905_at | — | — | — |
| 22 | 214052_x_at | BAT2D1 | 29 | 30 |
| 23 | 212520_s_at | SMARCA4 | 31 | 32 |
| 24 | 221419_s_at | — | — | — |
| 25 | 211948_x_at | BAT2D1 | 33 | 34 |
| 26 | 221860_at | HNRNPL | 35 | 36 |
| 27 | 211600_at | PTPRO | 37 | 38 |
| 28 | 214055_x_at | BAT2D1 | 39 | 40 |
| 29 | 220940_at | ANKRD36B | 41 | 42 |
| 30 | 212027_at | RBM25 | 43 | 44 |
| 31 | 213917_at | PAX8 | 45 | 46 |
| 32 | 208610_s_at | SRRM2 | 47 | 48 |
| 33 | 202379_s_at | NKTR | 49 | 50 |
| 34 | 211996_s_at | LOC100132247 | 51 | 52 |

*The Probeset ID refers to the identification no. of the Affymetrix HG-U133A Chip.

TABLE 2

| | | | SEQ ID No. | SEQ ID No. |
|---|---|---|---|---|
| No. | Probeset ID* | Gene Symbol | (mRNA) | (amino acid |
| 1 | 201554_x_at | GYG1 | 53 | 54 |
| 2 | 221449_s_at | ITFG1 | 55 | 56 |
| 3 | 201337_s_at | VAMP3 | 57 | 58 |
| 4 | 203207_s_at | MTFR1 | 59 | 60 |
| 5 | 214359_s_at | HSP90AB1 | 61 | 62 |
| 6 | 208029_s_at | LAPTM4B | 63 | 64 |
| 7 | 209739_s_at | PNPLA4 | 65 | 66 |
| 8 | 202226_s_at | CRK | 67 | 68 |
| 9 | 207124_s_at | GNB5 | 69 | 70 |
| 10 | 211450_s_at | MSH6 | 71 | 72 |
| 11 | 218163_at | MCTS1 | 73 | 74 |
| 12 | 218462_at | BXDC5 | 75 | 76 |
| 13 | 211563_s_at | C19orf2 | 77 | 78 |
| 14 | 215236_s_at | PICALM | 79 | 80 |
| 15 | 200973_s_at | TSPAN3 | 81 | 82 |
| 16 | 219819_s_at | MRPS28 | 83 | 84 |

*The Probeset ID refers to the identification no. of the Affymetrix HG-U133A Chip.

TABLE 3

| | | | SEQ ID No. | SEQ ID No. |
|---|---|---|---|---|
| No. | Probeset ID* | Gene Symbol | (mRNA) | (amino acid |
| 1 | 221872_at | RARRES1 | 85 | 86 |
| 2 | 211519_s_at | KIF2C | 87 | 88 |
| 3 | 219429_at | FA2H | 89 | 90 |
| 4 | 204259_at | MMP7 | 91 | 92 |

*The Probeset ID refers to the identification no. of the Affymetrix HG-U133A Chip.

TABLE 4

| | | | SEQ ID No. | SEQ ID No. |
|---|---|---|---|---|
| No. | Probeset ID* | Gene Symbol | (mRNA) | (amino acid |
| 1 | 206836_at | SLC6A3 | 93 | 94 |
| 2 | 208711_s_at | CCND1 | 95 | 96 |
| 3 | 221031_s_at | APOLD1 | 97 | 98 |
| 4 | 205903_s_at | KCNN3 | 99 | 100 |
| 5 | 205247_at | NOTCH4 | 101 | 102 |
| 6 | 219371_s_at | KLF2 | 103 | 104 |
| 7 | 204677_at | CDH5 | 105 | 106 |
| 8 | 205902_at | KCNN3 | 107 | 108 |
| 9 | 212558_at | SPRY1 | 109 | 110 |
| 10 | 221529_s_at | PLVAP | 111 | 112 |
| 11 | 212538_at | DOCK9 | 113 | 114 |
| 12 | 218995_s_at | EDN1 | 115 | 116 |
| 13 | 218353_at | RGS5 | 117 | 118 |
| 14 | 204468_s_at | TIE1 | 119 | 120 |
| 15 | 219091_s_at | MMRN2 | 121 | 122 |
| 16 | 205507_at | ARHGEF15 | 123 | 124 |
| 17 | 209070_s_at | RGS5 | 125 | 126 |
| 18 | 221489_s_at | SPRY4 | 127 | 128 |
| 19 | 203934_at | KDR | 129 | 130 |

*The Probeset ID refers to the identification no. of the Affymetrix HG-U133A Chip.

TABLE 5

| | | Gene |
|---|---|---|
| No. | Probeset ID* | Symbol |
| 1 | 202677_at | RASA1 |
| 2 | 207121_s_at | MAPK6 |
| 3 | 203218_at | MAPK9 |
| 4 | 200885_at | RHOC |
| 5 | 200059_s_at | RHOA |
| 6 | 218236_s_at | PRKD3 |
| 7 | 206702_at | TEK |
| 8 | 221016_s_at | TCF7L1 |
| 9 | 203238_s_at | NOTCH3 |
| 10 | 202273_at | PDGFRB |
| 11 | 205247_at | NOTCH4 |
| 12 | 32137_at | JAG2 |
| 13 | 204484_at | PIK3C2B |
| 14 | 202743_at | PIK3R3 |
| 15 | 205846_at | PTPRB |
| 16 | 203934_at | KDR |
| 17 | 202668_at | EFNB2 |
| 18 | 212099_at | RHOB |
| 19 | 219304_s_at | PDGFD |
| 20 | 210220_at | FZD2 |
| 21 | 204422_s_at | FGF2 |
| 22 | 202647_s_at | NRAS |
| 23 | 202095_s_at | BIRC5 |
| 24 | 219257_s_at | SPHK1 |
| 25 | 205962_at | PAK2 |
| 26 | 205897_at | NFATC4 |
| 27 | 208041_at | GRK1 |
| 28 | 208095_s_at | SRP72 |
| 29 | 200885_at | RHOC |
| 30 | 212294_at | GNG12 |
| 31 | 208736_at | ARPC3 |
| 32 | 217898_at | C15orf24 |
| 33 | 200059_s_at | RHOA |
| 34 | 207157_s_at | GNG5 |
| 35 | 208640_at | RAC1 |
| 36 | 201921_at | GNG10 |
| 37 | 209239_at | NFKB1 |
| 38 | 211963_s_at | ARPC5 |
| 39 | 204396_s_at | GRK5 |
| 40 | 201473_at | JUNB |
| 41 | 201466_s_at | JUN |
| 42 | 212099_at | RHOB |
| 43 | 202112_at | VWF |
| 44 | 213222_at | PLCB1 |
| 45 | 203896_s_at | PLCB4 |
| 46 | 202647_s_at | NRAS |
| 47 | 219918_s_at | ASPM |
| 48 | 217820_s_at | ENAH |
| 49 | 202647_s_at | NRAS |
| 50 | 205055_at | ITGAE |
| 51 | 200950_at | ARPC1A |
| 52 | 203065_s_at | CAV1 |
| 53 | 208750_s_at | ARF1 |
| 54 | 201659_s_at | ARL1 |
| 55 | 200059_s_at | RHOA |
| 56 | 201097_s_at | ARF4 |
| 57 | 204732_s_at | TRIM23 |
| 58 | 219431_at | ARHGAP10 |
| 59 | 209081_s_at | COL18A1 |
| 60 | 216264_s_at | LAMB2 |
| 61 | 210105_s_at | FYN |
| 62 | 204484_at | PIK3C2B |
| 63 | 202743_at | PIK3R3 |
| 64 | 204543_at | RAPGEF1 |
| 65 | 221180_at | YSK4 |
| 66 | 206044_s_at | BRAF |
| 67 | 217644_s_at | SOS2 |
| 68 | 206370_at | PIK3CG |
| 69 | 213475_s_at | ITGAL |
| 70 | 205718_at | ITGB7 |
| 71 | 221016_s_at | TCF7L1 |
| 72 | 205656_at | PCDH17 |
| 73 | 219656_at | PCDH12 |
| 74 | 204677_at | CDH5 |
| 75 | 204726_at | CDH13 |
| 76 | 208712_at | CCND1 |
| 77 | 213222_at | PLCB1 |
| 78 | 219427_at | FAT4 |
| 79 | 201921_at | GNG10 |
| 80 | 202981_x_at | SIAH1 |
| 81 | 201375_s_at | PPP2CB |
| 82 | 201218_at | CTBP2 |
| 83 | 200765_x_at | CTNNA1 |
| 84 | 208652_at | PPP2CA |
| 85 | 212294_at | GNG12 |
| 86 | 203896_s_at | PLCB4 |
| 87 | 220085_at | HELLS |
| 88 | 202468_s_at | CTNNAL1 |
| 89 | 206194_at | HOXC4 |
| 90 | 206858_s_at | HOXC6 |
| 91 | 201321_s_at | SMARCC2 |

*The Probeset ID refers to the identification no. of the Affymetrix HG-U133A Chip.

TABLE 6

| | | Grade | | Subtype/Cell | Clusters | also on |
|---|---|---|---|---|---|---|
| Chip # | Genevestigator_Chip Title | Thoenes | Stage | line | to Group | SNP array |
| 1 | RCC_clear cell_BI_rep1 | 2 | 2 | clear cell RCC | B | yes |
| 2 | RCC_clear cell_BI_rep2 | 2 | 2 | clear cell RCC | B | no |
| 3 | RCC_clear cell_BI_rep5 | 2 | 2 | clear cell RCC | A | no |
| 4 | RCC_clear cell_S1_BI_rep1 | 2 | 1 | clear cell RCC | C | yes |
| 5 | RCC_clear cell_S1_BI_rep2 | 1 | 2 | clear cell RCC | B | no |
| 6 | RCC_clear cell_S1_BI_rep3 | 2 | 2 | clear cell RCC | B | yes (out) |
| 7 | RCC_clear cell_S1_BI_rep4 | 1 | 1 | clear cell RCC | A | no |
| 8 | RCC_clear cell_S1_BI_rep5 | 2 | 2 | clear cell RCC | B | yes (out) |
| 9 | RCC_clear cell_S3_BI_rep1 | 3 | 3 | clear cell RCC | B | yes (out) |
| 10 | RCC_clear cell_S3_BI_rep2 | 2 | 3 | clear cell RCC | A | no |
| 11 | RCC_clear cell_S3_BI_rep3 | 3 | 2 | clear cell RCC | C | yes |
| 12 | RCC_clear cell_S3_BI_rep4 | 2 | 3 | clear cell RCC | C | yes |
| 13 | RCC_clear cell_S3_BI_rep5 | 2 | 3 | clear cell RCC | A | no |
| 14 | RCC_clear cell_S3_BI_rep6 | 3 | 3 | clear cell RCC | A | yes |
| 15 | RCC_clear cell_S4_BI_rep1 | 1 | 3 | clear cell RCC | C | yes |
| 16 | RCC_clear cell_S4_BI_rep2 | 1 | 3 | clear cell RCC | A | yes (out) |
| 17 | RCC_clear cell_S4_BI_rep3 | 2 | 3 | clear cell RCC | C | yes (out) |
| 18 | RCC_clear cell_S4_BI_rep4 | 2 | 3 | clear cell RCC | B | no |
| 19 | RCC_clear cell_S4_BI_rep5 | 2 | 3 | clear cell RCC | C | no |
| 20 | RCC_clear cell_S4_BI_rep6 | 2 | 2 | clear cell RCC | B | yes (out) |
| 21 | RCC_clear cell_BII_rep1 | 2 | 3 | clear cell RCC | B | yes (out) |
| 22 | RCC_clear cell_BII_rep2 | 2 | 2 | clear cell RCC | B | yes (out) |
| 23 | RCC_clear cell_BII_rep3 | 2 | 2 | clear cell RCC | B | yes |
| 24 | RCC_clear cell_BII_rep4 | 2 | 3 | clear cell RCC | A | no |
| 25 | RCC_clear cell_BII_rep5 | 1 | 1 | clear cell RCC | A | yes |
| 26 | RCC_clear cell_BII_rep6 | 2 | 2 | clear cell RCC | A | yes |
| 27 | RCC_clear cell_BII_rep7 | 2 | 2 | clear cell RCC | C | no |
| 28 | RCC_clear cell_BII_rep8 | 1 | 1 | clear cell RCC | A | yes |
| 29 | RCC_clear cell_BII_rep9 | 1 | 1 | clear cell RCC | A | yes |
| 30 | RCC_clear cell_BII_rep10 | 1 | 1 | clear cell RCC | A | yes |
| 31 | RCC_clear cell_BII_rep11 | 2 | 3 | clear cell RCC | A | yes |
| 32 | RCC_clear cell_BII_rep12 | 1 | 1 | chromophobe RCC | C | no |
| 33 | RCC_clear cell_BII_rep13 | 1 | 1 | clear cell RCC | A | no |
| 34 | RCC_clear cell_BII_rep14 | 2 | 1 | clear cell RCC | A | no |
| 35 | RCC_clear cell_BII_rep15 | 1 | 3 | clear cell RCC | A | yes |
| 36 | RCC_clear cell_BII_rep16 | 1 | 2 | clear cell RCC | A | no |
| 37 | RCC_clear cell_BII_rep17 | 2 | 3 | clear cell RCC | A | yes |
| 38 | RCC_clear cell_BII_rep18 | 1 | 1 | clear cell RCC | A | no |
| 39 | RCC_clear cell_BII_rep19 | 1 | 1 | clear cell RCC | A | yes (out) |
| 40 | RCC_clear cell_BII_rep20 | 1 | 1 | clear cell RCC | A | yes |
| 41 | RCC_clear cell_BII_rep21 | 2 | 1 | clear cell RCC | A | yes |
| 42 | RCC_clear cell_BII_rep22 | 1 | 1 | clear cell RCC | A | yes |
| 43 | RCC_clear cell_BII_rep23 | 2 | 1 | clear cell RCC | A | yes (out) |
| 44 | RCC_clear cell_BII_rep24 | 3 | 3 | clear cell RCC | C | yes (out) |
| 45 | RCC_clear cell_BII_rep25 | 1 | 1 | clear cell RCC and | C | yes |
| | | | | papillary RCC | | |
| 46 | RCC_clear cell_BII_rep26 | 1 | 1 | clear cell RCC | A | no |
| 47 | RCC_clear cell_BII_rep27 | 1 | 1 | clear cell RCC | A | yes (out) |
| 48 | RCC_clear cell_BII_rep28 | 2 | 2 | clear cell RCC | A | yes (out) |
| 49 | RCC_clear cell_BII_rep29 | 1 | 3 | clear cell RCC | A | no |
| 50 | RCC_clear cell_BII_rep30 | 1 | 1 | clear cell RCC | B | no |
| 51 | RCC_clear cell_BII_rep31 | 1 | 1 | clear cell RCC | A | no |
| 52 | RCC_clear cell_BII_rep32 | 2 | 3 | clear cell RCC | A | no |
| 53 | RCC_clear cell_BII_rep33 | 1 | 3 | clear cell RCC | C | no |
| 54 | RCC_clear cell_BII_rep34 | 1 | 3 | clear cell RCC | A | no |
| 55 | RCC_clear cell_BII_rep35 | — | — | Metastasis | B | no |
| 56 | RCC_clear cell_BII_rep36 | 1 | 1 | clear cell RCC | A | no |
| 57 | RCC_clear cell_BII_rep37 | 1 | 1 | clear cell RCC | A | no |
| 58 | RCC_clear cell_BII_rep38 | 2 | 3 | clear cell RCC | A | yes |
| 59 | RCC_clear cell_BII_rep39 | 2 | 2 | clear cell RCC | A | yes |
| 60 | RCC_clear cell_BII_rep40 | 2 | 2 | papillary RCC | C | yes (out) |
| 61 | RCC_clear cell_BII_rep41 | 2 | 3 | clear cell RCC | C | yes |
| 62 | RCC_clear cell_BII_rep42 | 1 | 1 | clear cell RCC | A | no |
| 63 | RCC_clear cell_S1_BII_rep1 | 1 | 2 | clear cell RCC | B | yes (out) |
| 64 | RCC_clear cell_S1_BII_rep2 | 1 | 1 | clear cell RCC | A | yes |
| 65 | RCC_clear cell_S1_BII_rep3 | 1 | 1 | clear cell RCC | A | yes |
| 66 | RCC_clear cell_S1_BII_rep4 | 1 | 1 | clear cell RCC | A | yes |
| 67 | RCC_clear cell_S1_BII_rep5 | 2 | 1 | clear cell RCC | A | yes |
| 68 | RCC_clear cell_S2_BII_rep1 | 1 | 2 | chromophobe RCC | B | yes |
| 69 | RCC_clear cell_S2_BII_rep2 | 1 | 2 | clear cell RCC | A | no |
| 70 | RCC_clear cell_S2_BII_rep3 | 1 | 2 | clear cell RCC | A | yes |
| 71 | RCC_clear cell_S3_BII_rep1 | 1 | 3 | clear cell RCC | B | no |
| 72 | RCC_clear cell_S3_BII_rep2 | 1 | 3 | clear cell RCC | B | no |
| 73 | RCC_clear cell_S3_BII_rep3 | 1 | 3 | clear cell RCC | A | no |
| 74 | RCC_clear cell_S3_BII_rep4 | 1 | 3 | clear cell RCC | A | yes |
| 75 | RCC_clear cell_S3_BII_rep5 | 2 | 3 | clear cell RCC | A | no |
| 76 | RCC_clear cell_S3_BII_rep6 | 1 | 3 | clear cell RCC | A | yes (out) |
| 77 | RCC_clear cell_S3_BII_rep7 | 2 | 3 | clear cell RCC | A | yes (out) |
| 78 | RCC_clear cell_S4_BII_rep1 | 1 | 3 | clear cell RCC | B | no |
| 79 | RCC_clear cell_S4_BII_rep2 | 1 | 1 | clear cell RCC | A | no |
| 80 | RCC_papillary_BI_rep1 | 2 | 2 | papillary RCC | A | no |
| 81 | RCC_papillary_BI_rep2 | 2 | 2 | papillary RCC | B | no |
| 82 | RCC_papillary_BI_rep3 | 1 | 1 | papillary RCC | C | yes |
| 83 | RCC_papillary_BI_rep5 | 2 | 3 | papillary RCC | C | no |
| 84 | RCC_papillary_BI_rep6 | 1 | 3 | papillary RCC | B | no |
| 85 | RCC_papillary_BI_rep7 | 2 | 2 | papillary RCC | B | no |
| 86 | RCC_papillary_BI_rep8 | 3 | 2 | papillary RCC | B | no |
| 87 | RCC_papillary_S1_BI_rep1 | 1 | 1 | papillary RCC | C | yes |
| 88 | RCC_papillary_S1_BI_rep2 | 1 | 1 | papillary RCC | C | yes |
| 89 | RCC_papillary_S2_BI_rep1 | 2 | 2 | papillary RCC | B | no |
| 90 | RCC_papillary_S2_BI_rep2 | 2 | 2 | papillary RCC | C | yes |
| 91 | RCC_papillary_S4_BI_rep1 | 2 | 3 | papillary RCC | C | yes |
| 92 | RCC_papillary_S4_BI_rep2 | 1 | 2 | papillary RCC | B | yes (out) |
| 93 | RCC_papillary_BII_rep1 | 2 | 3 | papillary RCC | C | yes |
| 94 | RCC_papillary_BII_rep2 | 1 | 2 | papillary RCC | C | yes |
| 95 | RCC_papillary_BII_rep3 | 1 | 1 | papillary RCC | C | yes |
| 96 | RCC_papillary_BII_rep4 | 1 | 1 | papillary RCC | C | no |
| 97 | RCC_papillary_BII_rep5 | 1 | 1 | papillary RCC | C | no |
| 98 | RCC_papillary_BII_rep6 | 1 | 1 | papillary RCC | C | no |
| 99 | RCC_papillary_BII_rep7 | 3 | 3 | clear cell RCC and | C | no |
| | | | | papillary RCC | | |
| 100 | RCC_meta._BI_rep1 | — | — | Metastasis | C | no |
| 101 | RCC_meta._BI_rep2 | — | — | Metastasis | A | no |
| 102 | RCC_meta._BI_rep3 | — | — | Metastasis | C | no |
| 103 | RCC_meta._BI_rep4 | — | — | Metastasis | C | no |
| 104 | RCC_meta._BI_rep5 | — | — | Metastasis | C | no |
| 105 | RCC_meta._BI_rep6 | — | — | Metastasis | C | no |
| 106 | RCC_meta._BI_rep7 | — | — | Metastasis | C | no |
| 107 | RCC_meta._BI_rep8 | — | — | Metastasis | C | no |
| 108 | RCC_meta._BI_rep9 | — | — | Metastasis | C | no |
| 109 | RCC_meta._BI_rep10 | — | — | Metastasis | C | no |
| 110 | RCC_meta._BI_rep11 | — | — | Metastasis | C | no |
| 111 | RCC_meta._BI_rep12 | — | — | Metastasis | C | no |
| 112 | RCC_meta._BI_rep13 | — | — | Metastasis | A | no |
| 113 | RCC_meta._BI_rep14 | — | — | Metastasis | A | no |
| 114 | RCC_cell line_BI_rep1 | — | — | UMRC2 | NA | no |
| 115 | RCC_cell line_BI_rep2 | — | — | SLR24 | NA | no |
| 116 | RCC_cell line_BI_rep3 | — | — | A-498 | NA | no |
| 117 | RCC_cell line_BI_rep4 | — | — | SK-RC 52 | NA | no |
| 118 | RCC_cell line_BI_rep5 | — | — | 786O (vhl19) | NA | no |
| 119 | RCC_cell line_BI_rep6 | — | — | UMRC 6 | NA | no |
| 120 | RCC_cell line_BI_rep7 | — | — | ACHN | NA | no |
| 121 | RCC_cell line_BI_rep8 | — | — | 786O (vhl30) | NA | no |
| 122 | RCC_cell line_BI_rep9 | — | — | A-704 | NA | no |
| 123 | RCC_cell line_BI_rep10 | — | — | SLR 26 | NA | no |
| 124 | RCC_cell line_BI_rep11 | — | — | Caki-1 | NA | no |
| 125 | RCC_cell line_BI_rep12 | — | — | RCC4 (vhl) | NA | no |
| 126 | RCC_cell line_BI_rep13 | — | — | 769-P | NA | no |
| 127 | RCC_cell line_BI_rep14 | — | — | KC 12 | NA | no |
| 128 | RCC_cell line_BI_rep15 | — | — | RCC4 (neo) | NA | no |
| 129 | RCC_cell line_BI_rep16 | — | — | SK-RC 29 | NA | no |
| 130 | RCC_cell line_BI_rep17 | — | — | SW 156 | NA | no |
| 131 | RCC_cell line_BI_rep18 | — | — | SK-RC 31 | NA | no |
| 132 | RCC_cell line_BI_rep19 | — | — | SLR 22 | NA | no |
| 133 | RCC_cell line_BI_rep20 | — | — | SK-RC 38 | NA | no |
| 134 | RCC_cell line_BI_rep21 | — | — | 786-O | NA | no |
| 135 | RCC_cell line_BI_rep22 | — | — | SK-RC 42 | NA | no |
| 136 | RCC_cell line_BI_rep23 | — | — | 786O | NA | no |
| 137 | RCC_cell line_meta._BI_rep1 | — | — | SLR 25 | NA | no |
| 138 | RCC_cell line_meta._BI_rep2 | — | — | SLR 20 | NA | no |
| 139 | RCC_cell line_meta._BI_rep3 | — | — | Caki-2 | NA | no |
| 140 | RCC_cell line_meta._BI_rep4 | — | — | SLR 21 | NA | no |
| 141 | RCC_cell line_meta._BI_rep5 | — | — | KU 19-20 | NA | no |
| 142 | RCC_cell line_meta._BI_rep6 | — | — | SLR 23 | NA | no |
| 143 | RCC_prost. can. cell line_BI_rep1 | — | — | PC3 hep 27 | NA | no |
| 144 | RCC_prost. can. cell line_BI_rep2 | — | — | PC3 hep 30 | NA | no |
| 145 | RCC_prost. can. cell line_BI_rep3 | — | — | PC3 vec 1 | NA | no |
| 146 | RCC_prost. can. cell line_BI_rep4 | — | — | PC3 vec 3 | NA | no |
| 147 | RCC_kidney cell line_BI_rep1 | — | — | HK-2 | NA | no |

TABLE 7

| | | | Percent |
|---|---|---|---|
| | | Percent | of gene |
| | | of gene | hit |
| | | hit | against |
| | | against | total Nr. |
| Pathway Name (Panther | Nr. of | total Nr. | pathway |
| Accession Nr.) | genes * | genes | hits |
| 2-arachidonoylglycerol | 2 | 0.2 | 0.4 |
| biosynthesis (P05726) | | | |
| 5-Hydroxytryptamine degredation | 1 | 0.1 | 0.2 |
| (P04372) | | | |
| 5HT1 type receptor mediated | 4 | 0.4 | 0.8 |
| signaling pathway (P04373) | | | |
| 5HT2 type receptor mediated | 10 | 1 | 2 |
| signaling pathway (P04374) | | | |
| 5HT3 type receptor mediated | 2 | 0.2 | 0.4 |
| signaling pathway (P04375) | | | |
| 5HT4 type receptor mediated | 4 | 0.4 | 0.8 |
| signaling pathway (P04376) | | | |
| Adenine and hypoxanthine salvage | 2 | 0.2 | 0.4 |
| pathway (P02723) | | | |
| Adrenaline and noradrenaline | 2 | 0.2 | 0.4 |
| biosynthesis (P00001) | | | |
| Alpha adrenergic receptor signaling | 6 | 0.6 | 1.2 |
| pathway (P00002) | | | |
| Alzheimer disease-amyloid | 6 | 0.6 | 1.2 |
| secretase pathway (P00003) | | | |
| Alzheimer disease-presenilin | 8 | 0.8 | 1.6 |
| pathway (P00004) | | | |
| Angiogenesis (P00005) | 21 | 2.2 | 4.3 |
| Angiotensin II-stimulated signaling | 4 | 0.4 | 0.8 |
| through G proteins and beta-arrestin | | | |
| (P05911) | | | |
| Apoptosis signaling pathway | 15 | 1.5 | 3 |
| (P00006) | | | |
| Axon guidance mediated by | 2 | 0.2 | 0.4 |
| Slit/Robo (P00008) | | | |
| Axon guidance mediated by netrin | 2 | 0.2 | 0.4 |
| (P00009) | | | |
| Axon guidance mediated by | 2 | 0.2 | 0.4 |
| semaphorins (P00007) | | | |
| B cell activation (P00010) | 12 | 1.2 | 2.4 |
| Beta1 adrenergic receptor signaling | 6 | 0.6 | 1.2 |
| pathway (P04377) | | | |
| Beta2 adrenergic receptor signaling | 6 | 0.6 | 1.2 |
| pathway (P04378) | | | |
| Beta3 adrenergic receptor signaling | 4 | 0.4 | 0.8 |
| pathway (P04379) | | | |
| Cadherin signaling pathway | 4 | 0.4 | 0.8 |
| (P00012) | | | |
| Cortocotropin releasing factor | 4 | 0.4 | 0.8 |
| receptor signaling pathway | | | |
| (P04380) | | | |
| Cytoskeletal regulation by Rho | 8 | 0.8 | 1.6 |
| GTPase (P00016) | | | |
| Dopamine receptor mediated | 7 | 0.7 | 1.4 |
| signaling pathway (P05912) | | | |
| EGF receptor signaling pathway | 10 | 1 | 2 |
| (P00018) | | | |
| Endogenous_cannabinoid_signaling | 2 | 0.2 | 0.4 |
| (P05730) | | | |
| Endothelin signaling pathway | 13 | 1.3 | 2.6 |
| (P00019) | | | |
| Enkephalin release (P05913) | 4 | 0.4 | 0.8 |
| FAS signaling pathway (P00020) | 6 | 0.6 | 1.2 |
| FGF signaling pathway (P00021) | 14 | 1.4 | 2.8 |
| Formyltetrahydroformate | 2 | 0.2 | 0.4 |
| biosynthesis (P02743) | | | |
| Heterotrimeric G-protein signaling | 12 | 1.2 | 2.4 |
| pathway-Gi alpha and Gs alpha | | | |
| mediated pathway (P00026) | | | |
| Heterotrimeric G-protein signaling | 12 | 1.2 | 2.4 |
| pathway-Gq alpha and Go alpha | | | |
| mediated pathway (P00027) | | | |
| Heterotrimeric G-protein signaling | 2 | 0.2 | 0.4 |
| pathway-rod outer segment | | | |
| phototransduction (P00028) | | | |
| Histamine H1 receptor mediated | 6 | 0.6 | 1.2 |
| signaling pathway (P04385) | | | |
| Histamine H2 receptor mediated | 2 | 0.2 | 0.4 |
| signaling pathway (P04386) | | | |
| Huntington disease (P00029) | 12 | 1.2 | 2.4 |
| Inflammation mediated by | 33 | 3.4 | 6.7 |
| Chemokine and cytokine signaling | | | |
| pathway (P00031) | | | |
| Insulin/IGF pathway-mitogen | 4 | 0.4 | 0.8 |
| activated protein kinase | | | |
| kinase/MAP kinase cascade | | | |
| (P00032) | | | |
| Insulin/IGF pathway-protein kinase | 2 | 0.2 | 0.4 |
| B signaling cascade (P00033) | | | |
| Integrin signalling pathway | 20 | 2.1 | 4.1 |
| (P00034) | | | |
| Interferon-gamma signaling | 4 | 0.4 | 0.8 |
| pathway (P00035) | | | |
| Interleukin signaling pathway | 8 | 0.8 | 1.6 |
| (P00036) | | | |
| Ionotropic glutamate receptor | 2 | 0.2 | 0.4 |
| pathway (P00037) | | | |
| JAK/STAT signaling pathway | 2 | 0.2 | 0.4 |
| (P00038) | | | |
| Metabotropic glutamate receptor | 2 | 0.2 | 0.4 |
| group I pathway (P00041) | | | |
| Metabotropic glutamate receptor | 4 | 0.4 | 0.8 |
| group II pathway (P00040) | | | |
| Metabotropic glutamate receptor | 6 | 0.6 | 1.2 |
| group III pathway (P00039) | | | |
| Methylcitrate cycle (P02754) | 2 | 0.2 | 0.4 |
| Muscarinic acetylcholine receptor 1 | 6 | 0.6 | 1.2 |
| and 3 signaling pathway (P00042) | | | |
| Muscarinic acetylcholine receptor 2 | 6 | 0.6 | 1.2 |
| and 4 signaling pathway (P00043) | | | |
| Nicotinic acetylcholine receptor | 6 | 0.6 | 1.2 |
| signaling pathway (P00044) | | | |
| Notch signaling pathway (P00045) | 2 | 0.2 | 0.4 |
| Opioid prodynorphin pathway | 4 | 0.4 | 0.8 |
| (P05916) | | | |
| Opioid proenkephalin pathway | 4 | 0.4 | 0.8 |
| (P05915) | | | |
| Opioid proopiomelanocortin | 4 | 0.4 | 0.8 |
| pathway (P05917) | | | |
| Oxidative stress response (P00046) | 13 | 1.3 | 2.6 |
| Oxytocin receptor mediated | 10 | 1 | 2 |
| signaling pathway (P04391) | | | |
| PDGF signaling pathway (P00047) | 11 | 1.1 | 2.2 |
| PI3 kinase pathway (P00048) | 4 | 0.4 | 0.8 |
| Parkinson disease (P00049) | 8 | 0.8 | 1.6 |
| Ras Pathway (P04393) | 6 | 0.6 | 1.2 |
| Synaptic_vesicle_trafficking | 2 | 0.2 | 0.4 |
| (P05734) | | | |
| T cell activation (P00053) | 10 | 1 | 2 |
| TCA cycle (P00051) | 2 | 0.2 | 0.4 |
| TGF-beta signaling pathway | 10 | 1 | 2 |
| (P00052) | | | |
| Thyrotropin-releasing hormone | 8 | 0.8 | 1.6 |
| receptor signaling pathway | | | |
| (P04394) | | | |
| Toll receptor signaling pathway | 2 | 0.2 | 0.4 |
| (P00054) | | | |
| Transcription regulation by bZIP | 2 | 0.2 | 0.4 |
| transcription factor (P00055) | | | |
| Ubiquitin proteasome pathway | 2 | 0.2 | 0.4 |
| (P00060) | | | |
| VEGF signaling pathway (P00056) | 11 | 1.1 | 2.2 |
| Wnt signaling pathway (P00057) | 18 | 1.8 | 3.7 |
| p38 MAPK pathway (P05918) | 1 | 0.1 | 0.2 |
| p53 pathway feedback loops 2 | 2 | 0.2 | 0.4 |
| (P04398) | | | |
| p53 pathway (P00059) | 8 | 0.8 | 1.6 |

* double value is shown, as candidates were blasted against Celera and NCBI (H. sapiens)

TABLE 8

| | Gene | | Gene | | Gene | | Gene |
|---|---|---|---|---|---|---|---|
| Angiogenesis | Symbol | Inflammation | Symbol | Integrin | Symbol | Wnt | Symbol |
| cluster I - | | cluster I - | | cluster I - | | cluster I - | |
| Probe Set ID* | | Probe Set ID* | | Probe Set ID* | | Probe Set ID* | |
| 202677_at | RASA1 | 205962_at | PAK2 | 217820_s_at | ENAH | 221016_s_at | TCF7L1 |
| 207121_s_at | MAPK6 | 205897_at | NFATC4 | 202647_s_at | NRAS | 205656_at | PCDH17 |
| 203218_at | MAPK9 | 208041_at | GRK1 | 205055_at | ITGAE | 219656_at | PCDH12 |
| 200885_at | RHOC | | | 200950_at | ARPC1A | 204677_at | CDH5 |
| 200059_s_at | RHOA | | | 203065_s_at | CAV1 | 204726_at | CDH13 |
| 218236_s_at | PRKD3 | | | | | 208712_at | CCND1 |
| | | | | | | 213222_at | PLCB1 |
| | | | | | | 219427_at | FAT4 |
| cluster II - | | cluster II - | | cluster II - | | cluster II - | |
| Probe Set ID* | | Probe Set ID* | | Probe Set ID* | | Probe Set ID* | |
| 206702_at | TEK | 208095_s_at | SRP72 | 208750_s_at | ARF1 | 201921_at | GNG10 |
| 221016_s_at | TCF7L1 | 200885_at | RHOC | 201659_s_at | ARL1 | 202981_x_at | SIAH1 |
| 203238_s_at | NOTCH3 | 212294_at | GNG12 | 200059_s_at | RHOA | 201375_s_at | PPP2CB |
| 202273_at | PDGFRB | 208736_at | ARPC3 | 201097_s_at | ARF4 | 201218_at | CTBP2 |
| 205247_at | NOTCH4 | 217898_at | C15orf24 | | | 200765_x_at | CTNNA1 |
| 32137_at | JAG2 | 200059_s_at | RHOA | | | 208652_at | PPP2CA |
| 204484_at | PIK3C2B | 207157_s_at | GNG5 | | | 212294_at | GNG12 |
| 202743_at | PIK3R3 | 208640_at | RAC1 | | | | |
| 205846_at | PTPRB | 201921_at | GNG10 | | | | |
| 203934_at | KDR | 209239_at | NFKB1 | | | | |
| 202668_at | EFNB2 | 211963_s_at | ARPC5 | | | | |
| 212099_at | RHOB | | | | | | |
| 219304_s_at | PDGFD | | | | | | |
| cluster III - | | cluster III - | | cluster III - | | cluster III - | |
| Probe Set ID* | | Probe Set ID* | | Probe Set ID* | | Probe Set ID* | |
| 210220_at | FZD2 | 204396_s_at | GRK5 | 204732_s_at | TRIM23 | 203896_s_at | PLCB4 |
| 204422_s_at | FGF2 | 201473_at | JUNB | 219431_at | ARHGAP10 | 220085_at | HELLS |
| 202647_s_at | NRAS | 201466_s_at | JUN | 209081_s_at | COL18A1 | 202468_s_at | CTNNAL1 |
| 202095_s_at | BIRC5 | 212099_at | RHOB | 216264_s_at | LAMB2 | | |
| 219257_s_at | SPHK1 | 202112_at | VWF | 210105_s_at | FYN | | |
| | | 213222_at | PLCB1 | 204484_at | PIK3C2B | | |
| | | | | 202743_at | PIK3R3 | | |
| | | cluster IV - | | cluster IV - | | cluster IV - | |
| | | Probe Set ID* | | Probe Set ID* | | Probe Set ID* | |
| | | 203896_s_at | PLCB4 | 204543_at | RAPGEF1 | 206194_at | HOXC4 |
| | | 202647_s_at | NRAS | 221180_at | YSK4 | 206858_s_at | HOXC6 |
| | | 219918_s_at | ASPM | 206044_s_at | BRAF | 201321_s_at | SMARCC2 |
| | | | | 217644_s_at | SOS2 | | |
| | | | | 206370_at | PIK3CG | | |
| | | | | 213475_s_at | ITGAL | | |
| | | | | 205718_at | ITGB7 | | |

*The Probeset ID refers to the identification no. of the Affymetrix HG-U133A Chip.

TABLE 9

| | Group A | | | Group B | | | Group C | | |
|---|---|---|---|---|---|---|---|---|---|
| | CD34 | DEK | MSH6 | CD34 | DEK | MSH6 | CD34 | DEK | MSH6 |
| Tumor | high | 1 | 2 | low | 3 | 0 | low | 0 | 0 |
| 1 | MVD | | | MVD | | | MVD | | |
| Tumor | high | 1 | 1 | high | 1 | 0 | low | 2 | 2 |
| 2 | MVD | | | MVD | | | MVD | | |
| Tumor | high | 2 | 2 | high | 0 | 0 | low | 1 | 1 |
| 3 | MVD | | | MVD | | | MVD | | |
| Tumor | high | 0 | 0 | high | 0 | 0 | low | 2 | 0 |
| 4 | MVD | | | MVD | | | MVD | | |
| Tumor | high | 1 | 1 | low | 0 | 0 | low | 0 | 0 |
| 5 | MVD | | | MVD | | | MVD | | |
| Tumor | high | 2 | 1 | low | 0 | 0 | low | 1 | 0 |
| 6 | MVD | | | MVD | | | MVD | | |
| Tumor | high | 2 | 2 | low | 0 | 0 | low | 2 | 2 |
| 7 | MVD | | | MVD | | | MVD | | |
| Tumor | high | 2 | 2 | low | 0 | 0 | low | 0 | 1 |
| 8 | MVD | | | MVD | | | MVD | | |
| Tumor | high | 1 | 2 | low | 0 | 0 | low | 2 | 2 |
| 9 | MVD | | | MVD | | | MVD | | |

0 = negative, 1 = weak staining intensity, 2 = moderate staining intensity, 3 = strong staining intensity

TABLE 10

| No. | ProbeSet ID | S | T* | Entrez | SEQ |
|---|---|---|---|---|---|
| 1 | 221860_at | 1 | 3.3441 | 3191 | 131 |
| 2 | 219754_at | 1 | 2.8455 | 55285 | 132 |
| 3 | 211454_x_at | 1 | 3.8185 | 400949 | 133 |
| 4 | 216112_at | 1 | 1.3782 | — | 134 |
| 5 | 211386_at | 1 | 1.8448 | 84786 | 135 |
| 6 | 33768_at | 1 | 1.5069 | 1762 | 136 |
| 7 | 206789_s_at | 1 | 2.0255 | 5451 | 137 |
| 8 | 215338_s_at | 1 | 2.9496 | 4820 | 138 |
| 9 | 32029_at | 1 | 2.4556 | 5170 | 139 |
| 10 | 212487_at | 1 | 2.8105 | 23131 | 140 |
| 11 | 222368_at | 1 | 2.0125 | — | 141 |
| 12 | 222366_at | 1 | 2.8874 | — | 142 |
| 13 | 204771_s_at | 1 | 3.2049 | 7270 | 143 |
| 14 | 213813_x_at | 1 | 2.8246 | — | 144 |
| 15 | 212783_at | 1 | 2.8134 | 5930 | 145 |
| 16 | 221191_at | 1 | 1.2696 | 54441 | 146 |
| 17 | 216527_at | 1 | 1.2548 | — | 147 |
| 18 | 215545_at | 1 | 1.2992 | — | 148 |
| 19 | 220905_at | 1 | 2.7221 | — | 149 |
| 20 | 208662_s_at | 1 | 3.4742 | 7267 | 150 |
| 21 | 208120_x_at | 1 | 3.3528 | 400949 | 151 |
| 22 | 48580_at | 1 | 2.9000 | 30827 | 152 |
| 23 | 213185_at | 1 | 1.6378 | 23247 | 153 |
| 24 | 203496_s_at | 1 | 2.7044 | 5469 | 154 |
| 25 | 203701_s_at | 1 | 1.6273 | 55621 | 155 |
| 26 | 207186_s_at | 1 | 3.5894 | 2186 | 156 |
| 27 | 219437_s_at | 1 | 3.5114 | 29123 | 157 |
| 28 | 212317_at | 1 | 2.7809 | 23534 | 158 |
| 29 | 217994_x_at | 1 | 3.1814 | 54973 | 159 |
| 30 | 210463_x_at | 1 | 1.6328 | 55621 | 160 |
| 31 | 212994_at | 1 | 2.7464 | 57187 | 161 |
| 32 | 202379_s_at | 1 | 4.4881 | 4820 | 162 |
| 33 | 219639_x_at | 1 | 2.9579 | 56965 | 163 |
| 34 | 205178_s_at | 1 | 2.9427 | 5930 | 164 |
| 35 | 215032_at | 1 | 1.8919 | 6239 | 165 |
| 36 | 213235_at | 1 | 2.7268 | 400506 | 166 |
| 37 | 210266_s_at | 1 | 2.9143 | 51592 | 167 |
| 38 | 203297_s_at | 1 | 2.0353 | 3720 | 168 |
| 39 | 212596_s_at | 1 | 2.1500 | 10042 | 169 |
| 40 | 218555_at | 1 | 1.3394 | 29882 | 170 |
| 41 | 202774_s_at | 1 | 2.6265 | 6433 | 171 |
| 42 | 214001_x_at | 1 | 2.6765 | — | 172 |
| 43 | 212571_at | 1 | 2.3779 | 57680 | 173 |
| 44 | 202682_s_at | 1 | 3.1557 | 7375 | 174 |
| 45 | 202473_x_at | 1 | 0.9283 | 3054 | 175 |
| 46 | 214464_at | 1 | 3.9210 | 8476 | 176 |
| 47 | 206567_s_at | 1 | 2.4514 | 51230 | 177 |
| 48 | 209579_s_at | 1 | 4.2461 | 8930 | 178 |
| 49 | 34260_at | 1 | 1.0468 | 9894 | 179 |
| 50 | 214195_at | 1 | 0.9257 | 1200 | 180 |
| 51 | 219105_x_at | 1 | 2.2929 | 23594 | 181 |
| 52 | 213328_at | 1 | 2.8602 | 4750 | 182 |
| 53 | 208663_s_at | 1 | 2.9901 | 7267 | 183 |
| 54 | 214843_s_at | 1 | 2.6647 | 23032 | 184 |
| 55 | 220072_at | 1 | 2.8119 | 79848 | 185 |
| 56 | 219468_s_at | 1 | 2.1238 | 404093 | 186 |
| 57 | 220370_s_at | 1 | 1.5578 | 57602 | 187 |
| 58 | 212318_at | 1 | 2.6286 | 23534 | 188 |
| 59 | 206169_x_at | 1 | 2.8966 | 23264 | 189 |
| 60 | 201728_s_at | 1 | 2.6719 | 9703 | 190 |
| 61 | 205434_s_at | 1 | 3.3218 | 22848 | 191 |
| 62 | 203597_s_at | 1 | 2.3050 | 11193 | 192 |
| 63 | 222291_at | 1 | 2.7298 | 25854 | 193 |
| 64 | 208859_s_at | 1 | 2.8255 | 546 | 194 |
| 65 | 201959_s_at | 1 | 3.4533 | 23077 | 195 |
| 66 | 40569_at | 1 | 1.2913 | 7593 | 196 |
| 67 | 209088_s_at | 1 | 3.1422 | 29855 | 197 |
| 68 | 209945_s_at | 1 | 2.6078 | 2932 | 198 |
| 69 | 206967_at | 1 | 1.4198 | 904 | 199 |
| 70 | 206416_at | 1 | 1.4064 | 7755 | 200 |
| 71 | 205367_at | 1 | 1.2617 | 10603 | 201 |
| 72 | 222186_at | 1 | 1.7502 | 54469 | 202 |
| 73 | 208936_x_at | 1 | 2.4216 | 3964 | 203 |
| 74 | 202102_s_at | 1 | 3.5148 | 23476 | 204 |
| 75 | 213971_s_at | 1 | 2.1596 | 100292841 | 205 |
| 76 | 201680_x_at | 1 | 3.3522 | 51593 | 206 |
| 77 | 213876_x_at | 1 | 2.1010 | 8233 | 207 |
| 78 | 221350_at | 1 | 0.2892 | 3224 | 208 |
| 79 | 216525_x_at | 1 | 2.8062 | 5387 | 209 |
| 80 | 222182_s_at | 1 | 3.4695 | 4848 | 210 |
| 81 | 214473_x_at | 1 | 2.7307 | 5387 | 211 |
| 82 | 208475_at | 1 | 0.9177 | 55691 | 212 |
| 83 | 215667_x_at | 1 | 2.4823 | 100132832 | 213 |
| 84 | 219392_x_at | 1 | 4.3567 | 55771 | 214 |
| 85 | 213205_s_at | 1 | 0.3487 | 23132 | 215 |
| 86 | 222047_s_at | 1 | 3.9224 | 51593 | 216 |
| 87 | 209932_s_at | 1 | 3.8225 | 1854 | 217 |
| 88 | 219507_at | 1 | 2.6101 | 51319 | 218 |
| 89 | 204538_x_at | 1 | 4.8527 | 9284 | 219 |
| 90 | 41386_i_at | 1 | 1.0805 | 23135 | 220 |
| 91 | 214004_s_at | 1 | 4.1868 | 9686 | 221 |
| 92 | 217804_s_at | 1 | 2.7713 | 3609 | 222 |
| 93 | 216751_at | 1 | 0.6769 | — | 223 |
| 94 | 215541_s_at | 1 | 0.7933 | 1729 | 224 |
| 95 | 212028_at | 1 | 2.5778 | 58517 | 225 |
| 96 | 217576_x_at | 1 | 0.7751 | 6655 | 226 |
| 97 | 215434_x_at | 1 | 3.6643 | 100132406 | 227 |
| 98 | 212759_s_at | 1 | 2.7559 | 6934 | 228 |
| 99 | 45687_at | 1 | 3.0172 | 78994 | 229 |
| 100 | 209534_x_at | 1 | 3.0095 | 11214 | 230 |
| 101 | 213956_at | 1 | 2.3039 | 9857 | 231 |
| 102 | 202384_s_at | 1 | 1.9349 | 6949 | 232 |
| 103 | 220940_at | 1 | 3.7431 | 57730 | 233 |
| 104 | 216550_x_at | 1 | 2.9023 | 23253 | 234 |
| 105 | 201224_s_at | 1 | 2.8594 | 10250 | 235 |
| 106 | 220696_at | 1 | −0.7982 | — | 236 |
| 107 | 206565_x_at | 1 | 3.2219 | 11039 | 237 |
| 108 | 213650_at | 1 | 3.8542 | 23015 | 238 |
| 109 | 204403_x_at | 1 | 4.2212 | 9747 | 239 |
| 110 | 201856_s_at | 1 | 1.8053 | 51663 | 240 |
| 111 | 210069_at | 1 | 2.6025 | 1375 | 241 |
| 112 | 202574_s_at | 1 | 1.6621 | 1455 | 242 |
| 113 | 204741_at | 1 | 2.5709 | 636 | 243 |
| 114 | 218920_at | 1 | 2.4642 | 54540 | 244 |
| 115 | 221526_x_at | 1 | 2.9858 | 56288 | 245 |
| 116 | 208930_s_at | 1 | 3.8138 | 3609 | 246 |
| 117 | 204428_s_at | 1 | 1.7085 | 3931 | 247 |
| 118 | 214041_x_at | 1 | 3.0486 | 6168 | 248 |
| 119 | 221043_at | 1 | 0.7752 | — | 249 |
| 120 | 212451_at | 1 | 2.5922 | 9728 | 250 |
| 121 | 218808_at | 1 | 0.0000 | 55152 | 251 |
| 122 | 213311_s_at | 1 | 4.0840 | 22980 | 252 |
| 123 | 44146_at | 1 | 1.6473 | 26205 | 253 |
| 124 | 205415_s_at | 1 | 0.7316 | 4287 | 254 |
| 125 | 213729_at | 1 | 3.6076 | 55660 | 255 |
| 126 | 217734_s_at | 1 | 2.7312 | 11180 | 256 |
| 127 | 205339_at | 1 | 0.0537 | 6491 | 257 |
| 128 | 221718_s_at | 1 | 2.9493 | 11214 | 258 |
| 129 | 39650_s_at | 1 | 0.5091 | 80003 | 259 |
| 130 | 221496_s_at | 1 | 2.8841 | 10766 | 260 |
| 131 | 210094_s_at | 1 | 3.0740 | 56288 | 261 |
| 132 | 214526_x_at | 1 | 2.3316 | 5379 | 262 |
| 133 | 214723_x_at | 1 | 1.7216 | 375248 | 263 |
| 134 | 209715_at | 1 | 3.3266 | 23468 | 264 |
| 135 | 212177_at | 1 | 4.3567 | 25957 | 265 |
| 136 | 217679_x_at | 1 | 3.9197 | — | 266 |
| 137 | 213850_s_at | 1 | 4.4362 | 9169 | 267 |
| 138 | 216563_at | 1 | 2.3194 | 23253 | 268 |
| 139 | 202818_s_at | 1 | 2.9712 | 6924 | 269 |
| 140 | 221829_s_at | 1 | 4.5874 | 3842 | 270 |
| 141 | 220368_s_at | 1 | 1.4496 | 55671 | 271 |
| 142 | 210666_at | 1 | 1.3500 | 3423 | 272 |
| 143 | 211342_x_at | 1 | 2.5036 | 9968 | 273 |
| 144 | 216450_x_at | 1 | 3.3900 | 7184 | 274 |
| 145 | 212926_at | 1 | 2.0261 | 23137 | 275 |
| 146 | 208995_s_at | 1 | 2.0054 | 9360 | 276 |
| 147 | 217152_at | 1 | 0.6767 | — | 277 |
| 148 | 213277_at | 1 | −0.6754 | 677 | 278 |
| 149 | 222104_x_at | 1 | 4.0594 | 2967 | 279 |
| 150 | 215279_at | 1 | 0.9778 | — | 280 |
| 151 | 217620_s_at | 1 | 1.3042 | 5291 | 281 |
| 152 | 218742_at | 1 | 1.2959 | 64428 | 282 |
| 153 | 207605_x_at | 1 | 1.2134 | 51351 | 283 |
| 154 | 210579_s_at | 1 | −0.3495 | 10107 | 284 |
| 155 | 208803_s_at | 1 | 2.7603 | 6731 | 285 |
| 156 | 44822_s_at | 1 | 0.8390 | 54531 | 286 |
| 157 | 214870_x_at | 1 | 4.9662 | 10028844 | 287 |
| 158 | 205787_x_at | 1 | 2.0152 | 9877 | 288 |
| 159 | 213893_x_at | 1 | 2.7065 | 5383 | 289 |
| 160 | 48612_at | 1 | 1.5062 | 9683 | 290 |
| 161 | 222133_s_at | 1 | 1.4355 | 51105 | 291 |
| 162 | 212027_at | 1 | 3.5596 | 58517 | 292 |
| 163 | 222024_s_at | 1 | 3.4062 | 11214 | 293 |
| 164 | 208993_s_at | 1 | 3.0181 | 9360 | 294 |
| 165 | 205370_x_at | 1 | 4.7353 | 1629 | 295 |
| 166 | 222193_at | 1 | 0.2730 | 60526 | 296 |
| 167 | 214035_x_at | 1 | 4.8781 | 399491 | 297 |
| 168 | 201861_s_at | 1 | 4.8281 | 9208 | 298 |
| 169 | 208797_s_at | 1 | 1.1428 | 23015 | 299 |
| 170 | 204195_s_at | 1 | 0.9807 | 5316 | 300 |
| 171 | 222034_at | 1 | 1.6939 | 10399 | 301 |
| 172 | 220828_s_at | 1 | −0.4009 | 55338 | 302 |
| 173 | 208900_s_at | 1 | 3.6447 | 7150 | 303 |
| 174 | 205134_s_at | 1 | 1.5062 | 26747 | 304 |
| 175 | 216310_at | 1 | 1.3131 | 57551 | 305 |
| 176 | 201205_at | 1 | 0.5415 | 100292328 | 306 |
| 177 | 201996_s_at | 1 | 2.0236 | 23013 | 307 |
| 178 | 221501_x_at | 1 | 5.0156 | 339047 | 308 |
| 179 | 216843_x_at | 1 | 2.3442 | 5379 | 309 |
| 180 | 208879_x_at | 1 | 1.4191 | 24148 | 310 |
| 181 | 43544_at | 1 | 1.8638 | 10025 | 311 |
| 182 | 204909_at | 1 | −0.1882 | 1656 | 312 |
| 183 | 202509_s_at | 1 | 2.2336 | 7127 | 313 |
| 184 | 214395_x_at | 1 | 1.9902 | 1936 | 314 |
| 185 | 215582_x_at | 1 | 0.9031 | 8888 | 315 |
| 186 | 220796_x_at | 1 | 4.0349 | 79939 | 316 |
| 187 | 206323_x_at | 1 | 3.5161 | 4983 | 317 |
| 188 | 209136_s_at | 1 | 2.1393 | 9100 | 318 |
| 189 | 218859_s_at | 1 | 2.6581 | 51575 | 319 |
| 190 | 216212_s_at | 1 | 2.1806 | 1736 | 320 |
| 191 | 220071_x_at | 1 | 4.0476 | 55142 | 321 |
| 192 | 208994_s_at | 1 | 2.5538 | 9360 | 322 |
| 193 | 204227_s_at | 1 | 1.1780 | 7084 | 323 |
| 194 | 202773_s_at | 1 | 0.6752 | 6433 | 324 |
| 195 | 222351_at | 1 | 1.8154 | 5519 | 325 |
| 196 | 58900_at | 1 | 1.6313 | 222070 | 326 |
| 197 | 206056_x_at | 1 | 4.4435 | 6693 | 327 |
| 198 | 210251_s_at | 1 | 3.0237 | 22902 | 328 |
| 199 | 203468_at | 1 | 2.9207 | 8558 | 329 |
| 200 | 211289_x_at | 1 | 2.1424 | 728642 | 330 |
| 201 | 214052_x_at | 1 | 2.7074 | 23215 | 331 |
| 202 | 204649_at | 1 | −0.3037 | 10024 | 332 |
| 203 | 219380_x_at | 1 | 1.4667 | 5429 | 333 |
| 204 | 215848_at | 1 | 0.4399 | 49855 | 334 |
| 205 | 207598_x_at | 1 | 1.8445 | 7516 | 335 |
| 206 | 217644_s_at | 1 | 0.6763 | 6655 | 336 |
| 207 | 222249_at | 1 | −0.5170 | — | 337 |
| 208 | 218914_at | 1 | 1.2460 | 51093 | 338 |
| 209 | 212620_at | 1 | 1.2975 | 23060 | 339 |
| 210 | 208989_s_at | 1 | 1.7391 | 22992 | 340 |
| 211 | 202821_s_at | 1 | 2.0807 | 4026 | 341 |
| 212 | 213926_s_at | 1 | 2.2782 | 3267 | 342 |
| 213 | 215856_at | 1 | 0.3196 | 284266 | 343 |
| 214 | 32032_at | 1 | 2.2692 | 8220 | 344 |
| 215 | 201072_s_at | 1 | 2.1603 | 6599 | 345 |
| 216 | 208710_s_at | 1 | 3.9944 | 8943 | 346 |
| 217 | 200702_s_at | 1 | 3.8224 | 57062 | 347 |
| 218 | 217485_x_at | 1 | 2.1485 | 5379 | 348 |
| 219 | 213526_s_at | 1 | 1.3571 | 55957 | 349 |
| 220 | 220456_at | 1 | 1.6757 | 55304 | 350 |
| 221 | 214756_x_at | 1 | 2.0706 | 5379 | 351 |
| 222 | 214353_at | 1 | 0.2380 | — | 352 |
| 223 | 78495_at | 1 | 1.6266 | 155060 | 353 |
| 224 | 203204_s_at | 1 | 1.2374 | 9682 | 354 |
| 225 | 217878_s_at | 1 | 2.0496 | 996 | 355 |
| 226 | 41160_at | 1 | 2.1791 | 53615 | 356 |
| 227 | 214017_s_at | 1 | 0.0077 | 9704 | 357 |
| 228 | 214659_x_at | 1 | 2.2679 | 56252 | 358 |
| 229 | 50376_at | 1 | 2.4008 | 55311 | 359 |
| 230 | 216187_x_at | 1 | 4.4533 | — | 360 |
| 231 | 213445_at | 1 | 1.5876 | 23144 | 361 |
| 232 | 217611_at | 1 | 0.4124 | 157697 | 362 |
| 233 | 205068_s_at | 1 | 2.7417 | 23092 | 363 |
| 234 | 201635_s_at | 1 | 2.8099 | 8087 | 364 |
| 235 | 214552_s_at | 1 | 0.8368 | 9135 | 365 |
| 236 | 220962_s_at | 1 | 0.4547 | 29943 | 366 |
| 237 | 221780_s_at | 1 | 2.2823 | 55661 | 367 |
| 238 | 211097_s_at | 1 | −0.1281 | 5089 | 368 |
| 239 | 217622_at | 1 | 0.2285 | 25807 | 369 |
| 240 | 201026_at | 1 | 1.6815 | 9669 | 370 |
| 241 | 211996_s_at | 1 | 4.9230 | 100132247 | 371 |
| 242 | 220609_at | 1 | 1.7994 | 202181 | 372 |
| 243 | 213344_s_at | 1 | −0.0550 | 3014 | 373 |
| 244 | 207205_at | 1 | −0.3542 | 1089 | 374 |
| 245 | 206966_s_at | 1 | 0.0713 | 11278 | 375 |
| 246 | 208610_s_at | 1 | 4.7898 | 23524 | 376 |
| 247 | 204097_s_at | 1 | 2.7474 | 51634 | 377 |
| 248 | 211948_x_at | 1 | 4.0689 | 23215 | 378 |
| 249 | 212885_at | 1 | 2.7556 | 10199 | 379 |
| 250 | 37278_at | 1 | 0.8022 | 6901 | 380 |
| 251 | 206500_s_at | 1 | 0.8385 | 55320 | 381 |
| 252 | 214055_x_at | 1 | 4.4115 | 23215 | 382 |
| 253 | 214501_s_at | 1 | 3.9227 | 9555 | 383 |
| 254 | 214335_at | 1 | 0.1607 | 6141 | 384 |
| 255 | AFFX-M27830_5_at | 1 | 3.7141 | — | 385 |
| 256 | 221023_s_at | 1 | −0.6003 | 81033 | 386 |
| 257 | 217654_at | 1 | −0.0306 | — | 387 |
| 258 | 220466_at | 1 | 0.3488 | 80071 | 388 |
| 259 | 215605_at | 1 | 0.8272 | 10499 | 389 |
| 260 | 46142_at | 1 | 0.9219 | 64788 | 390 |
| 261 | 201024_x_at | 1 | 4.6527 | 9669 | 391 |
| 262 | 202301_s_at | 1 | 2.7863 | 65117 | 392 |
| 263 | 202414_at | 1 | 2.6600 | 2073 | 393 |
| 264 | 211886_s_at | 1 | −0.6122 | 6910 | 394 |
| 265 | 217380_s_at | 1 | −0.4102 | 7511 | 395 |
| 266 | 214250_at | 1 | 0.4289 | 4926 | 396 |
| 267 | 214911_s_at | 1 | 4.4278 | 6046 | 397 |
| 268 | 208685_x_at | 1 | 4.2214 | 6046 | 398 |
| 269 | 214693_x_at | 1 | 4.9733 | 100132406 | 399 |
| 270 | 214742_at | 1 | 0.5155 | 22994 | 400 |
| 271 | 222023_at | 1 | −0.9535 | 11214 | 401 |
| 272 | 202339_at | 1 | 1.4628 | 8189 | 402 |
| 273 | 203792_x_at | 1 | −0.0612 | 7703 | 403 |
| 274 | 221686_s_at | 1 | −0.1091 | 9400 | 404 |
| 274 | 221686_s_at | 1 | −0.1091 | 9400 | 404 |
| 275 | 212079_s_at | 1 | 1.6697 | 4297 | 405 |
| 276 | 208237_x_at | 1 | 0.1915 | 53616 | 406 |
| 277 | 221683_s_at | 1 | 1.1982 | 80184 | 407 |
| 278 | 217471_at | 1 | −0.9988 | — | 408 |
| 279 | 212106_at | 1 | 2.3823 | 23197 | 409 |
| 280 | 217498_at | 1 | −0.9042 | — | 410 |
| 281 | 220401_at | 1 | −1.0337 | 79860 | 411 |
| 282 | 81737_at | 1 | −0.4582 | — | 412 |
| 283 | 219897_at | 1 | 0.6897 | 79845 | 413 |
| 284 | 221007_s_at | 1 | 2.0715 | 81608 | 414 |
| 285 | 207349_s_at | 1 | −0.1878 | 7352 | 415 |
| 286 | 214113_s_at | −1 | 1.0133 | 9939 | 416 |
| 287 | 202919_at | −1 | 1.1561 | 25843 | 417 |
| 288 | 219485_s_at | −1 | 0.7641 | 5716 | 418 |
| 289 | 216304_x_at | −1 | 1.0802 | 10730 | 419 |
| 290 | 217959_s_at | −1 | 1.2672 | 51399 | 420 |
| 291 | 214429_at | −1 | 1.1991 | 9107 | 421 |
| 292 | 201020_at | −1 | 1.1169 | 7533 | 422 |
| 293 | 200056_s_at | −1 | 1.0703 | 10438 | 423 |
| 294 | 209551_at | −1 | 0.1229 | 84272 | 424 |
| 295 | 212268_at | −1 | 0.4909 | 1992 | 425 |
| 296 | 208992_s_at | −1 | 1.2101 | 6774 | 426 |
| 297 | 217865_at | −1 | 1.9469 | 55819 | 427 |
| 298 | 212833_at | −1 | 0.9751 | 91137 | 428 |
| 299 | 218449_at | −1 | 0.9798 | 55325 | 429 |
| 300 | 221531_at | −1 | −0.2519 | 80349 | 430 |
| 301 | 203156_at | −1 | 1.2641 | 11215 | 431 |
| 302 | 213027_at | −1 | 1.1477 | 6738 | 432 |
| 303 | 221547_at | −1 | 0.2353 | 8559 | 433 |
| 304 | 209096_at | −1 | 0.7868 | 7336 | 434 |
| 305 | 212461_at | −1 | 1.1393 | 51582 | 435 |
| 306 | 202166_s_at | −1 | −0.0224 | 5504 | 436 |
| 307 | 201176_s_at | −1 | 2.1289 | 372 | 437 |
| 308 | 212815_at | −1 | 0.5212 | 10973 | 438 |
| 309 | 219819_s_at | −1 | 0.0455 | 28957 | 439 |
| 310 | 212573_at | −1 | 0.9699 | 23052 | 440 |
| 311 | 202381_at | −1 | 1.9412 | 8754 | 441 |
| 312 | 202194_at | −1 | 2.0624 | 50999 | 442 |
| 313 | 201351_s_at | −1 | 1.2193 | 10730 | 443 |
| 314 | 203136_at | −1 | 1.4305 | 10567 | 444 |
| 315 | 211703_s_at | −1 | −0.6144 | 83941 | 445 |
| 316 | 209786_at | −1 | 0.7245 | 10473 | 446 |
| 317 | 214545_s_at | −1 | −0.8808 | 11212 | 447 |
| 318 | 204342_at | −1 | 0.5450 | 29957 | 448 |
| 319 | 212335_at | −1 | 0.9285 | 2799 | 449 |
| 320 | 202089_s_at | −1 | −0.3621 | 25800 | 450 |
| 321 | 200698_at | −1 | 1.9150 | 11014 | 451 |
| 322 | 219162_s_at | −1 | 0.3746 | 65003 | 452 |
| 323 | 203376_at | −1 | 0.7431 | 51362 | 453 |
| 324 | 218042_at | −1 | 1.3650 | 51138 | 454 |
| 325 | 213750_at | −1 | −0.0803 | 26156 | 455 |
| 326 | 220199_s_at | −1 | 0.7265 | 64853 | 456 |
| 327 | 217786_at | −1 | −0.0522 | 10419 | 457 |
| 328 | 203646_at | −1 | −0.0231 | 2230 | 458 |
| 329 | 208761_s_at | −1 | 1.3619 | 7341 | 459 |
| 330 | 202579_x_at | −1 | 1.6123 | 10473 | 460 |
| 331 | 208841_s_at | −1 | 1.7403 | 9908 | 461 |
| 332 | 218616_at | −1 | −0.4623 | 57117 | 462 |
| 333 | 217919_s_at | −1 | 0.6034 | 28977 | 463 |
| 334 | 212418_at | −1 | 0.7882 | 1997 | 464 |
| 335 | 212038_s_at | −1 | 2.2340 | 7416 | 465 |
| 336 | 203142_s_at | −1 | 0.5976 | 8546 | 466 |
| 337 | 201078_at | −1 | 1.5492 | 9375 | 467 |
| 338 | 202979_s_at | −1 | −0.0674 | 58487 | 468 |
| 339 | 209330_s_at | −1 | 1.4640 | 3184 | 469 |
| 340 | 218578_at | −1 | 0.0056 | 79577 | 470 |
| 341 | 209861_s_at | −1 | 1.0650 | 10988 | 471 |
| 342 | 200991_s_at | −1 | 1.0796 | 9784 | 472 |
| 343 | 202675_at | −1 | 1.2146 | 6390 | 473 |
| 344 | 218570_at | −1 | 0.2103 | 114971 | 474 |
| 345 | 208944_at | −1 | 2.1295 | 7048 | 475 |
| 346 | 200071_at | −1 | 1.0260 | 10285 | 476 |
| 347 | 211676_s_at | −1 | 0.7779 | 3459 | 477 |
| 348 | 203776_at | −1 | 0.3872 | 27238 | 478 |
| 349 | 221381_s_at | −1 | 1.1420 | 10933 | 479 |
| 350 | 209112_at | −1 | 1.4732 | 1027 | 480 |
| 351 | 209310_s_at | −1 | 0.7831 | 837 | 481 |
| 352 | 203261_at | −1 | 1.7458 | 10671 | 482 |
| 353 | 208860_s_at | −1 | −0.4157 | 546 | 483 |
| 354 | 206174_s_at | −1 | 0.8336 | 5537 | 484 |
| 355 | 212168_at | −1 | 0.5727 | 10137 | 485 |
| 356 | 201529_s_at | −1 | 0.6302 | 6117 | 486 |
| 357 | 212438_at | −1 | 0.3453 | 11017 | 487 |
| 358 | 212544_at | −1 | 1.0899 | 9326 | 488 |
| 359 | 203689_s_at | −1 | 1.1102 | 2332 | 489 |
| 360 | 201179_s_at | −1 | 0.2789 | 2773 | 490 |
| 361 | 208857_s_at | −1 | 1.1374 | 5110 | 491 |
| 362 | 203138_at | −1 | 0.5301 | 8520 | 492 |
| 363 | 202799_at | −1 | 1.0538 | 8192 | 493 |
| 364 | 218519_at | −1 | 0.0973 | 55032 | 494 |
| 365 | 218486_at | −1 | 0.8606 | 8462 | 495 |
| 366 | 203758_at | −1 | 1.9611 | 1519 | 496 |
| 367 | 211967_at | −1 | 2.2047 | 114908 | 497 |
| 368 | 208029_s_at | −1 | 0.5497 | 55353 | 498 |
| 369 | 201408_at | −1 | 1.3267 | 5500 | 499 |
| 370 | 218395_at | −1 | 0.8451 | 64431 | 500 |
| 371 | 200973_s_at | −1 | 0.0428 | 10099 | 501 |
| 372 | 200983_x_at | −1 | 2.2568 | 966 | 502 |
| 373 | 204045_at | −1 | 0.6250 | 9338 | 503 |
| 374 | 211985_s_at | −1 | 1.4396 | 801 | 504 |
| 375 | 213882_at | −1 | −0.5182 | 83941 | 505 |
| 376 | 205084_at | −1 | 0.1770 | 55973 | 506 |
| 377 | 200777_s_at | −1 | 2.5773 | 9689 | 507 |
| 378 | 213883_s_at | −1 | 1.3376 | 83941 | 508 |
| 379 | 212536_at | −1 | 0.9452 | 23200 | 509 |
| 380 | 212515_s_at | −1 | 0.9260 | 1654 | 510 |
| 381 | 200628_s_at | −1 | 0.7853 | 7453 | 511 |
| 382 | 213405_at | −1 | 0.5778 | 57403 | 512 |
| 383 | 209296_at | −1 | 1.3937 | 5495 | 513 |
| 384 | 218229_s_at | −1 | 0.8658 | 57645 | 514 |
| 385 | 218946_at | −1 | 1.8086 | 27247 | 515 |
| 386 | 202823_at | −1 | 0.9882 | 6921 | 516 |
| 387 | 208666_s_at | −1 | 0.5861 | 6767 | 517 |
| 388 | 201689_s_at | −1 | −0.0148 | 7163 | 518 |
| 389 | 201716_at | −1 | 0.8628 | 6642 | 519 |
| 390 | 218137_s_at | −1 | 0.8201 | 60682 | 520 |
| 391 | 200054_at | −1 | 0.4418 | 8882 | 521 |
| 392 | 208638_at | −1 | 2.6546 | 10130 | 522 |
| 393 | 206542_s_at | −1 | 1.1986 | 6595 | 523 |
| 394 | 209208_at | −1 | −0.2471 | 9526 | 524 |
| 395 | 218185_s_at | −1 | 0.6604 | 55156 | 525 |
| 396 | 209300_s_at | −1 | 0.3792 | 25977 | 526 |
| 397 | 214531_s_at | −1 | 0.5117 | 6642 | 527 |
| 398 | 209027_s_at | −1 | 0.6943 | 10006 | 528 |
| 399 | 200876_s_at | −1 | 2.7394 | 5689 | 529 |
| 400 | 221808_at | −1 | 1.2999 | 9367 | 530 |
| 401 | 200812_at | −1 | 1.8392 | 10574 | 531 |
| 402 | 217898_at | −1 | 2.4200 | 56851 | 532 |
| 403 | 213404_s_at | −1 | 1.7281 | 6009 | 533 |
| 404 | 217313_at | −1 | 1.0846 | — | 534 |
| 405 | 208852_s_at | −1 | 1.5732 | 821 | 535 |
| 406 | 205961_s_at | −1 | 0.3967 | 11168 | 536 |
| 407 | 218408_at | −1 | 0.7486 | 26519 | 537 |
| 408 | 202978_s_at | −1 | 0.0798 | 58487 | 538 |
| 409 | 214812_s_at | −1 | 2.4679 | 55233 | 539 |
| 410 | 212878_s_at | −1 | 0.1008 | 3831 | 540 |
| 411 | 202119_s_at | −1 | 2.0419 | 8895 | 541 |
| 412 | 209387_s_at | −1 | 0.3916 | 4071 | 542 |
| 413 | 209440_at | −1 | 2.0282 | 5631 | 543 |
| 414 | 220985_s_at | −1 | 0.4970 | 81790 | 544 |
| 415 | 218172_s_at | −1 | 0.4654 | 79139 | 545 |
| 416 | 203284_s_at | −1 | 0.6826 | 9653 | 546 |
| 417 | 202163_s_at | −1 | 0.3248 | 9337 | 547 |
| 418 | 216483_s_at | −1 | 0.6811 | 56005 | 548 |
| 419 | 212887_at | −1 | 1.2028 | 10484 | 549 |
| 420 | 206989_s_at | −1 | 1.7013 | 9169 | 550 |
| 421 | 217725_x_at | −1 | 1.4834 | 26135 | 551 |
| 422 | 202314_at | −1 | 0.2208 | 1595 | 552 |
| 423 | 202680_at | −1 | 0.3848 | 2961 | 553 |
| 424 | 217843_s_at | −1 | 1.1394 | 29079 | 554 |
| 425 | 209025_s_at | −1 | 0.9337 | 10492 | 555 |
| 426 | 200668_s_at | −1 | 2.8886 | 7323 | 556 |
| 427 | 210691_s_at | −1 | 0.4321 | 27101 | 557 |
| 428 | 201472_at | −1 | 1.6450 | 7411 | 558 |
| 429 | 212956_at | −1 | 0.8992 | 23158 | 559 |
| 430 | 220926_s_at | −1 | 0.1949 | 80267 | 560 |
| 431 | 219356_s_at | −1 | 1.6198 | 51510 | 561 |
| 432 | 201511_at | −1 | 0.8429 | 14 | 562 |
| 433 | 212453_at | −1 | 1.5109 | 26128 | 563 |
| 434 | 212440_at | −1 | 1.7380 | 11017 | 564 |
| 435 | 218236_s_at | −1 | 0.8765 | 23683 | 565 |
| 436 | 201515_s_at | −1 | 2.1329 | 7247 | 566 |
| 437 | 201858_s_at | −1 | 1.1537 | 5552 | 567 |
| 438 | 212250_at | −1 | 1.6486 | 92140 | 568 |
| 439 | 217900_at | −1 | 2.1840 | 55699 | 569 |
| 440 | 217989_at | −1 | 1.7563 | 51170 | 570 |
| 441 | 210250_x_at | −1 | 1.3997 | 158 | 571 |
| 442 | 218761_at | −1 | 1.1344 | 54778 | 572 |
| 443 | 203053_at | −1 | 1.5515 | 10286 | 573 |
| 444 | 203721_s_at | −1 | 1.2257 | 51096 | 574 |
| 445 | 212989_at | −1 | 0.3137 | 259230 | 575 |
| 446 | 201847_at | −1 | 2.1432 | 3988 | 576 |
| 447 | 203983_at | −1 | 1.0034 | 7257 | 577 |
| 448 | 221761_at | −1 | 0.5938 | 159 | 578 |
| 449 | 203302_at | −1 | 0.3182 | 1633 | 579 |
| 450 | 212112_s_at | −1 | 1.9386 | 23673 | 580 |
| 451 | 210283_x_at | −1 | 1.2625 | 10605 | 581 |
| 452 | 217987_at | −1 | 1.4309 | 54529 | 582 |
| 453 | 218118_s_at | −1 | 0.6889 | 10431 | 583 |
| 454 | 202832_at | −1 | 1.3710 | 9648 | 584 |

“No.” refers to gene numbers of Table 10 as mentioned herein. “ProbeSetID” refers to the identification number on the Affymetrix gene chip HT_HG-U133A. “S” refers to “side”. The “side” defines whether a gene has to be over- or underexpressed for state B according to the model described in the Example section under “3. Identification of RCC specific gene sets”. The value “1” indicates an overexpression and the value “−1” indicates underexpression. “T*” refers to “threshold” and describes the value which used as control to decide on overexpression or underexpression. It corresponds to threshold θ_{g} in equation (3) of example 3, “Entrez” describes the Entrez Genbank accession number. “SEQ” refers to the SEQ ID No..

TABLE 11

| No | ProbeSet ID | S | T* | Entrez | SEQ |
|---|---|---|---|---|---|
| 1 | 203744_at | 1 | 0.4538 | 3149 | 585 |
| 2 | 208699_x_at | 1 | 2.1223 | 7086 | 586 |
| 3 | 218847_at | 1 | 0.7209 | 10644 | 587 |
| 4 | 203355_s_at | 1 | 0.1877 | 23362 | 588 |
| 5 | 213009_s_at | 1 | 1.3696 | 4591 | 589 |
| 6 | 219874_at | 1 | −0.3138 | 84561 | 590 |
| 7 | 218412_s_at | 1 | 1.9539 | 9569 | 591 |
| 8 | 214039_s_at | 1 | 3.7728 | 55353 | 592 |
| 9 | 208905_at | 1 | 3.8095 | 54205 | 593 |
| 10 | 201870_at | 1 | 1.2113 | 10953 | 594 |
| 11 | 34764_at | 1 | 0.3539 | 23395 | 595 |
| 12 | 212186_at | 1 | 1.2865 | 31 | 596 |
| 13 | 218526_s_at | 1 | 1.4536 | 29098 | 597 |
| 14 | 202515_at | 1 | 2.3460 | 1739 | 598 |
| 15 | 222056_s_at | 1 | 1.1711 | 51011 | 599 |
| 16 | 217852_s_at | 1 | 3.1994 | 55207 | 600 |
| 17 | 222165_x_at | 1 | 0.2755 | 79095 | 601 |
| 18 | 221196_x_at | 1 | 0.7029 | 79184 | 602 |
| 19 | 206836_at | −1 | 2.3095 | 6531 | 603 |
| 20 | 208712_at | −1 | 2.9253 | 595 | 604 |
| 21 | 221747_at | −1 | 2.6506 | 7145 | 605 |
| 22 | 208711_s_at | −1 | 3.0413 | 595 | 606 |
| 23 | 218864_at | −1 | 0.6244 | 7145 | 607 |
| 24 | 205247_at | −1 | 0.9875 | 4855 | 608 |
| 25 | 219232_s_at | −1 | 2.6881 | 112399 | 609 |
| 26 | 222033_s_at | −1 | 2.7751 | 2321 | 610 |
| 27 | 205902_at | −1 | −0.5815 | 3782 | 611 |
| 28 | 208981_at | −1 | 2.8818 | 5175 | 612 |
| 29 | 204468_s_at | −1 | 0.3388 | 7075 | 613 |
| 30 | 218995_s_at | −1 | 0.6571 | 1906 | 614 |
| 31 | 221529_s_at | −1 | 2.6064 | 83483 | 615 |
| 32 | 202112_at | −1 | 3.0937 | 7450 | 616 |
| 33 | 212171_x_at | −1 | 3.1837 | 7422 | 617 |
| 34 | 210513_s_at | −1 | 2.6802 | 7422 | 618 |
| 35 | 204736_s_at | −1 | −0.0441 | 1464 | 619 |
| 36 | 215244_at | −1 | 0.1464 | 26220 | 620 |
| 37 | 204726_at | −1 | 0.7609 | 1012 | 621 |
| 38 | 221009_s_at | −1 | 2.4870 | 51129 | 622 |
| 39 | 209652_s_at | −1 | 0.3797 | 5228 | 623 |
| 40 | 221794_at | −1 | 1.0626 | 57572 | 624 |
| 41 | 219134_at | −1 | 2.1160 | 64123 | 625 |
| 42 | 204677_at | −1 | 2.2375 | 1003 | 626 |
| 43 | 221031_s_at | −1 | 1.6851 | 81575 | 627 |
| 44 | 205073_at | −1 | 1.7351 | 1573 | 628 |
| 45 | 209071_s_at | −1 | 4.2320 | 8490 | 629 |
| 46 | 210287_s_at | −1 | −0.8701 | 2321 | 630 |
| 47 | 203934_at | −1 | 2.2038 | 3791 | 631 |
| 48 | 210869_s_at | −1 | 3.0257 | 4162 | 632 |
| 49 | 214297_at | −1 | −0.6537 | 1464 | 633 |
| 50 | 206481_s_at | −1 | 1.9796 | 9079 | 634 |
| 51 | 206236_at | −1 | −0.0285 | 2828 | 635 |
| 52 | 205507_at | −1 | 0.4425 | 22899 | 636 |
| 53 | 218484_at | −1 | 1.9968 | 56901 | 637 |
| 54 | 219656_at | −1 | 0.8195 | 51294 | 638 |
| 55 | 218353_at | −1 | 4.3356 | 8490 | 639 |
| 56 | 218950_at | −1 | 0.6994 | 64411 | 640 |
| 57 | 208982_at | −1 | 3.2519 | 5175 | 641 |
| 58 | 209784_s_at | −1 | 0.8851 | 3714 | 642 |
| 59 | 203421_at | −1 | −0.1158 | 9537 | 643 |
| 60 | 208394_x_at | −1 | 2.5754 | 11082 | 644 |
| 61 | 211626_x_at | −1 | 1.1110 | 2078 | 645 |
| 62 | 211527_x_at | −1 | 2.4324 | 7422 | 646 |
| 63 | 209439_s_at | −1 | 1.7810 | 5256 | 647 |
| 64 | 209086_x_at | −1 | 1.6992 | 4162 | 648 |
| 65 | 213075_at | −1 | 1.9951 | 169611 | 649 |
| 66 | 218723_s_at | −1 | 2.6944 | 28984 | 650 |
| 67 | 221489_s_at | −1 | 1.5033 | 81848 | 651 |
| 68 | 209070_s_at | −1 | 3.0697 | 8490 | 652 |
| 69 | 213792_s_at | −1 | 2.9646 | 3643 | 653 |
| 70 | 218825_at | −1 | 0.4534 | 51162 | 654 |
| 71 | 40687_at | −1 | 1.7953 | 2701 | 655 |
| 72 | 221123_x_at | −1 | 2.2593 | 55893 | 656 |
| 73 | 55583_at | −1 | 0.8311 | 57572 | 657 |
| 74 | 214438_at | −1 | 0.3285 | 3142 | 658 |
| 75 | 205656_at | −1 | 2.0638 | 27253 | 659 |
| 76 | 205572_at | −1 | 1.2492 | 285 | 660 |
| 77 | 206271_at | −1 | 1.7322 | 7098 | 661 |
| 78 | 218149_s_at | −1 | 2.9141 | 55893 | 662 |
| 79 | 211266_s_at | −1 | −0.6392 | 2828 | 663 |
| 80 | 205903_s_at | −1 | −1.5187 | 3782 | 664 |
| 81 | 32137_at | −1 | 0.9339 | 3714 | 665 |
| 82 | 204642_at | −1 | 1.3247 | 1901 | 666 |
| 83 | 44783_s_at | −1 | 1.7356 | 23462 | 667 |
| 84 | 207414_s_at | −1 | 0.0035 | 5046 | 668 |
| 85 | 213030_s_at | −1 | 0.3669 | 5362 | 669 |
| 86 | 205199_at | −1 | 1.5983 | 768 | 670 |
| 87 | 202479_s_at | −1 | 1.4550 | 28951 | 671 |
| 88 | 202878_s_at | −1 | 2.8315 | 22918 | 672 |
| 89 | 218804_at | −1 | −0.1414 | 55107 | 673 |
| 90 | 209543_s_at | −1 | 2.1103 | 947 | 674 |
| 91 | 219091_s_at | −1 | 2.1372 | 79812 | 675 |
| 92 | 209200_at | −1 | 1.5685 | 4208 | 676 |
| 93 | 201578_at | −1 | 2.5705 | 5420 | 677 |
| 94 | 204464_s_at | −1 | 1.5208 | 1909 | 678 |
| 95 | 210512_s_at | −1 | 4.3501 | 7422 | 679 |
| 96 | 206995_x_at | −1 | 0.5703 | 8578 | 680 |
| 97 | 52255_s_at | −1 | −0.0854 | 50509 | 681 |
| 98 | 219315_s_at | −1 | 2.0402 | 79652 | 682 |
| 99 | 210078_s_at | −1 | 0.1896 | 7881 | 683 |
| 100 | 218731_s_at | −1 | 2.3146 | 64856 | 684 |
| 101 | 212382_at | −1 | 1.9947 | 6925 | 685 |
| 102 | 212977_at | −1 | 1.7593 | 57007 | 686 |
| 103 | 215104_at | −1 | −0.3730 | 83714 | 687 |
| 104 | 212793_at | −1 | 0.1560 | 23500 | 688 |
| 105 | 206814_at | −1 | −0.1826 | 4803 | 689 |
| 106 | 201655_s_at | −1 | 2.4592 | 3339 | 690 |
| 107 | 200878_at | −1 | 4.2720 | 2034 | 691 |
| 108 | 203438_at | −1 | 1.1001 | 8614 | 692 |
| 109 | 203238_s_at | −1 | 3.3351 | 4854 | 693 |
| 110 | 212538_at | −1 | 1.4583 | 23348 | 694 |
| 111 | 213349_at | −1 | 2.0507 | 23023 | 695 |
| 112 | 212758_s_at | −1 | 1.7063 | 6935 | 696 |
| 113 | 204904_at | −1 | 1.5891 | 2701 | 697 |
| 114 | 208851_s_at | −1 | 1.9715 | 7070 | 698 |
| 115 | 221814_at | −1 | 1.0068 | 25960 | 699 |
| 116 | 213541_s_at | −1 | 0.5653 | 2078 | 700 |
| 117 | 219821_s_at | −1 | −0.0525 | 54438 | 701 |
| 118 | 218507_at | −1 | 3.3707 | 29923 | 702 |
| 119 | 204200_s_at | −1 | 0.5029 | 5155 | 703 |
| 120 | 218839_at | −1 | 0.8679 | 23462 | 704 |
| 121 | 221748_s_at | −1 | 3.7428 | 7145 | 705 |
| 122 | 222079_at | −1 | −0.8229 | 2078 | 706 |
| 123 | 201328_at | −1 | 1.9460 | 2114 | 707 |
| 124 | 201041_s_at | −1 | 4.0892 | 1843 | 708 |
| 125 | 212951_at | −1 | 2.1755 | 221395 | 709 |
| 126 | 202478_at | −1 | 1.6323 | 28951 | 710 |
| 127 | 211148_s_at | −1 | 0.0334 | 285 | 711 |
| 128 | 207290_at | −1 | −1.5986 | 5362 | 712 |
| 129 | 47550_at | −1 | 0.6652 | 11178 | 713 |
| 130 | 38918_at | −1 | 0.2916 | 9580 | 714 |
| 131 | 212387_at | −1 | 2.0779 | 6925 | 715 |
| 132 | 205846_at | −1 | 0.5781 | 5787 | 716 |
| 133 | 209183_s_at | −1 | 2.8982 | 11067 | 717 |
| 134 | 203753_at | −1 | 2.4717 | 6925 | 718 |
| 135 | 204463_s_at | −1 | 0.4644 | 1909 | 719 |
| 136 | 205326_at | −1 | 0.9484 | 10268 | 720 |
| 137 | 209199_s_at | −1 | 1.7644 | 4208 | 721 |
| 138 | 212386_at | −1 | 2.8700 | 6925 | 722 |
| 139 | 219619_at | −1 | 0.8413 | 54769 | 723 |
| 140 | 218660_at | −1 | 2.1403 | 8291 | 724 |
| 141 | 201624_at | −1 | 2.7292 | 1615 | 725 |
| 142 | 218975_at | −1 | −0.5101 | 50509 | 726 |
| 143 | 219700_at | −1 | 0.4445 | 57125 | 727 |
| 144 | 213891_s_at | −1 | 2.3933 | 6925 | 728 |
| 145 | 201809_s_at | −1 | 2.5137 | 2022 | 729 |
| 146 | 202877_s_at | −1 | 1.9466 | 22918 | 730 |
| 147 | 205935_at | −1 | −0.3196 | 2294 | 731 |
| 148 | 203063_at | −1 | 0.4574 | 9647 | 732 |
| 149 | 217844_at | −1 | 2.5577 | 58190 | 733 |
| 150 | 200632_s_at | −1 | 4.0630 | 10397 | 734 |
| 151 | 201365_at | −1 | 1.9308 | 4947 | 735 |
| 152 | 220027_s_at | −1 | 1.1731 | 54922 | 736 |
| 153 | 222146_s_at | −1 | 2.3658 | 6925 | 737 |
| 154 | 200904_at | −1 | 3.7534 | 3133 | 738 |
| 155 | 41856_at | −1 | 0.3849 | 219699 | 739 |
| 156 | 207560_at | −1 | 0.4660 | 9154 | 740 |
| 157 | 220335_x_at | −1 | 0.9676 | 23491 | 741 |
| 158 | 218876_at | −1 | 0.3678 | 51673 | 742 |
| 159 | 219777_at | −1 | 2.1049 | 474344 | 743 |
| 160 | 205341_at | −1 | −0.9784 | 30846 | 744 |
| 161 | 212813_at | −1 | 2.0963 | 83700 | 745 |
| 162 | 219761_at | −1 | 0.1239 | 51267 | 746 |
| 163 | 209438_at | −1 | 1.2005 | 5256 | 747 |
| 164 | 212730_at | −1 | 1.0922 | 23336 | 748 |
| 165 | 214265_at | −1 | 0.3480 | 8516 | 749 |
| 166 | 204134_at | −1 | 0.8627 | 5138 | 750 |
| 167 | 200795_at | −1 | 3.4708 | 8404 | 751 |
| 168 | 218892_at | −1 | −1.2529 | 8642 | 752 |
| 169 | 202912_at | −1 | 3.5054 | 133 | 753 |
| 170 | 221870_at | −1 | 2.5560 | 30846 | 754 |
| 171 | 212599_at | −1 | 2.5884 | 26053 | 755 |
| 172 | 208850_s_at | −1 | 1.5384 | 7070 | 756 |
| 173 | 206477_s_at | −1 | −1.2220 | 4858 | 757 |
| 174 | 45297_at | −1 | 1.2774 | 30846 | 758 |
| 175 | 201150_s_at | −1 | 2.9579 | 7078 | 759 |
| 176 | 38671_at | −1 | 1.8357 | 23129 | 760 |
| 177 | 218656_s_at | −1 | 2.0210 | 10186 | 761 |
| 178 | 212552_at | −1 | 3.2921 | 3241 | 762 |
| 179 | 213869_x_at | −1 | 2.4380 | 7070 | 763 |
| 180 | 219602_s_at | −1 | 0.1530 | 63895 | 764 |
| 181 | 208983_s_at | −1 | 1.1928 | 5175 | 765 |
| 182 | 212235_at | −1 | 2.0713 | 23129 | 766 |
| 183 | 205801_s_at | −1 | 1.0815 | 25780 | 767 |
| 184 | 219719_at | −1 | −0.9314 | 51751 | 768 |
| 185 | 204220_at | −1 | 1.6400 | 9535 | 769 |
| 186 | 212494_at | −1 | 1.3189 | 23371 | 770 |
| 187 | 220471_s_at | −1 | −0.7667 | 80177 | 771 |
| 188 | 336_at | −1 | −0.5174 | 6915 | 772 |
| 189 | 211340_s_at | −1 | 2.6655 | 4162 | 773 |
| 190 | 222101_s_at | −1 | 1.1035 | 8642 | 774 |
| 191 | 220507_s_at | −1 | 0.3747 | 51733 | 775 |
| 192 | 203439_s_at | −1 | 0.3593 | 8614 | 776 |
| 193 | 212226_s_at | −1 | 3.8913 | 8613 | 777 |
| 194 | 218805_at | −1 | 1.8552 | 55340 | 778 |
| 195 | 64064_at | −1 | 1.6477 | 55340 | 779 |

“No.” refers to gene numbers of Table 10 as mentioned herein. “ProbeSetID” refers to the identification number on the Affymetrix gene chip HT_HG-U133A. “S” refers to “side”. The “side” defines whether a gene has to be over- or underexpressed in state C according to the model described in the Example section under “3. Identification of RCC specific gene sets”. The value “1” indicates an overexpression and the value “−1” indicates underexpression. “T*” refers to “threshold” and describes the value which used as control to decide on overexpression or underexpression. It corresponds to threshold θ_{g} in equation (3) of example 3. “Entrez” describes the Entrez Genbank accession number. “SEQ” refers to the SEQ ID No..

The following publications were considered in the context of the invention:
1. D. Hanahan, R. A. Weinberg, Cell 100, 57 (2000).
2. M. Baudis, M. L. Cleary, Bioinformatics 17, 1228 (2001).
3. L. J. Engle, C. L. Simpson, J. E. Landers, Oncogene 25, 1594 (2006).
4. R. Beroukhim et al., Cancer Res 69, 4674 (2009).
5. R. Beroukhim et al., Nature 463, 899 (2010).
6. R. S. Huang, M. E. Dolan, Pharmacogenomics 11, 471 (2010).
7. J. L. Huret, S. Senon, A. Bernheim, P. Dessen, Cell Mol Biol (Noisy-le-grand) 50, 805 (2004).
8. M. Baudis, BMC Cancer 7, 226 (2007).
9. F. Forozan, R. Karhu, J. Kononen, A. Kallioniemi, O. P. Kallioniemi, Trends in Genetics 13, 405 (1997).
10. T. Hruz et al., Adv Bioinformatics 2008, 420747 (2008).
11. P. Zimmermann, L. Hennig, W. Gruissem, Trends Plant Sci 10, 407 (2005).
12. V. G. Tusher, R. Tibshirani, G. Chu, Proc Natl Acad Sci USA 98, 5116 (2001).
13. G. M. Poage et al., PLoS One 5, e9651 (2010).
14. W. Thoenes, S. Storkel, H. J. Rumpelt, Pathol Res Pract 181, 125 (1986).
15. J. N. Eble, G. Sauter, E. J. I., I. A. E. Sesterhenn, World Health Organization Classification of Tumours. Pathology and Genetics of Tumours of the Urinary System and Male Genital Organs. (IARC Press, Lyon, 2004).
16. H. Bengtsson, P. Wirapati, T. P. Speed, Bioinformatics 25, 2149 (2009).
17. H. Bengtsson, A. Ray, P. Spellman, T. P. Speed, Bioinformatics 25, 861 (2009).
18. E. S. Venkatraman, A. B. Olshen, Bioinformatics 23, 657 (2007).
19. A. J. Iafrate et al., Nat Genet 36, 949 (2004).
20. P. D. Thomas et al., Genome Res 13, 2129 (2003).
21. H. Mi et al., Nucleic Acids Res 33, D284 (2005).
22. R. C. Gentleman et al., Genome Biol 5, R80 (2004).
23. A. I. Saeed et al., Methods Enzymol 411, 134 (2006).
24. J. Kononen et al., Nat Med 4, 844 (1998).
25. M. A. Rubin, R. Dunn, M. Strawderman, K. J. Pienta, Am J Surg Pathol 26, 312 (2002).
26. P. Schraml et al., J Pathol 196, 186 (2002).
27. Cheng Li and Wing Hung Wong, Proc. Natl. Acad. Sci. Vol. 98, 31-36 (2001a).
28. Cheng Li and Wing Hung Wong, Genome Biology 2(8) (2001b).
29. [dChip] dChip Software, available at http://www.dchip.org.
30. Tusher V G, Tibshirani R, Chu G., Proc Natl Acad Sci USA/98(9): 5116-5121 (2001).
31. T. Martinetz and K. Schulten. A, Artificial Neural Networks, I:397-402, (1991).
32. hanging Fan, Irene Gijbels: Local Polynomial Modelling and Its Applications. Chapman and Hall/CRC, 1996, ISBN 978-0412983214.
33. Clarke, R., Ressom, H. W., Wang, A., Xuan, J., Liu, M. C., Gehan, E. A., & Wang, Y., Nature Reviews Cancer, 8(1), 37-49, (2008).
34. Gene Expression Omnibus: http://www.ncbi.nlm.nih.gov/geo/35.
35. Gregory Shakhnarovich, Trevor Darrell, Piotr Indyk: Nearest-neighbor methods in learning and vision. MIT Press, 2005, ISBN 026219547X

1. A method of diagnosing, prognosing, stratifying and/or screening renal cell carcinoma (RCC) in at least one human or animal patient suspected of being afflicted with RCC comprising the steps of:
(a) providing a sample of a human or animal individual suspected to suffer from RCC;
(b) testing the sample for a signature indicative of a discrete RCC-specific state by determining expression of at least 6 genes of Table 10; and
(c) allocating a discrete RCC-specific state to the sample based on the signature determined in step (b).

2. A method of determining the responsiveness of at least one human or animal individual suspected of being afflicted with RCC towards a pharmaceutically active agent comprising the steps of:
(a) providing a sample of a human or animal individual suspected to suffer from RCC before the pharmaceutically active agent is administered;
(b) testing the sample for a signature indicative of a discrete RCC-specific state by determining expression of at least 6 genes of Table 10;
(c) allocating a discrete RCC-specific state to the sample based on the signature determined in step (b);
(d) determining the effects of the pharmaceutically active agent on the disease symptoms in the individual; and
(e) identifying a correlation between the effects on disease symptoms and/or discrete RCC-specific states and the initial discrete RCC-specific state of the sample.

3. A method of predicting the responsiveness of at least one patient suspected of being afflicted with RCC towards a pharmaceutically active agent comprising the steps of:
(a) determining whether a correlation exists between effects on disease symptoms and/or discrete RCC-specific states and the initial discrete RCC-specific state as a consequence of administration of a pharmaceutically active agent by using the method of claim 2;
(b) testing a sample of a human or animal individual patient suspected of being afflicted with RCC for a signature indicative of a discrete RCC-specific state by determining expression of at least 6 genes of Table 10;
(c) allocating a discrete and a discrete RCC specific state-specific state to said sample based on the signature determined in step (c);
(d) comparing the discrete and a discrete RCC specific state-specific state of the sample in step (c) vs. the discrete and a discrete RCC specific state-specific state for which a correlation has been determined in step (a); and
(e) predicting the effect of a pharmaceutically active compound on the disease symptoms in the patient.

4. A method of determining the effects of a potential pharmaceutically active compound for treatment of RCC, comprising the steps of:
(a) providing a sample of a human or animal individual suspected to suffer from RCC before a pharmaceutically active agent is applied;
(b) testing the sample from step (a) for a signature indicative of a discrete RCC-specific state by determining expression of at least 6 genes of Table 10;
(c) allocating a discrete RCC-specific state to the sample from step (a) based on the signature determined in step (b);
(d) providing a sample of the same human or animal individual suspected to suffer from RCC after a pharmaceutically active agent is applied;
(e) testing the sample from step (d) for a signature indicative of a discrete RCC-specific state by determining expression of at least 6 genes of Table 10;
(f) allocating a discrete RCC-specific state to the sample from step (d) based on the signature determined in step (e); and
(g) comparing the discrete RCC-specific states identified in steps (c) and (f).

5. The method of claim 1, wherein the signature is characterized by the expression pattern of at least 10 genes of Table 10 with genes 1 to 286 of Table 10 being overexpressed and genes 287 to 454 of Table 10 being underexpressed.

6. The method of claim 1, wherein the signature is characterized by the expression pattern of at least 10 genes of Table 10 with genes 1 to 286 of Table 10 being underexpressed and genes 287 to 454 of Table 10 being overexpressed.

7. The method of claim 6, wherein the signature can be further subdivided by determining expression of at least 6 genes of Table 11.

8. The method of claim 7, wherein the signature is characterized by the expression pattern of at least 10 genes of Table 11 with genes 1 to 19 of Table 11 being overexpressed and genes 20 to 195 of Table 11 being underexpressed.

9. The method of claim 7, wherein the signature is characterized by the expression pattern of at least 10 genes of Table 11 with genes 1 to 19 of Table 11 being underexpressed and genes 20 to 195 of Table 11 being overexpressed.

10. A signature which is defined by the expression pattern of at least 6 genes of Table 10 for use in diagnosing, prognosing, stratifying and/or screening renal cell cancer in human or animal individuals.

11. A signature which is defined by the expression pattern of at least 6 genes of Table 10 for use as a read out of a target for development, identification and/or screening of at least one pharmaceutically active compound for treatment of renal cell cancer.

12. The signature for use according to claim 10, which is defined by the expression pattern of at least 6 genes of Table 10 with genes 1 to 286 of Table 10 being overexpressed and genes 287 to 454 of Table 10 being underexpressed.

13. The signature for use according to claim 10, which is defined by the expression pattern of at least 6 genes of Table 10 with genes 1 to 286 of Table 10 being underexpressed and genes 287 to 454 of Table 10 being overexpressed, and which is further defined by the expression pattern of at least 6 genes of Table 11.

14. The signature for use according to claim 13, wherein genes 1 to 19 of Table 11 are overexpressed and genes 20 to 195 of Table 11 are underexpressed.

15. The signature for use according to claim 13, wherein genes 1 to 19 of Table 11 are underexpressed and genes 20 to 195 of Table 11 are overexpressed.
